(19) United States
(12) Patent Application Publication
(10) Pub. No.: US 2012/0078160 A1
(43) Pub. Date: Mar. 29, 2012

(54) METHOD AND APPARATUS FOR CANCER THERAPY

(75) Inventor: Kathleen McMillan, Concord, MA (US)

(73) Assignee: Gradiant Research, LLC, Concord, MA (US)

(21) Appl. No.: 13/225,073

(22) Filed: Sep. 2, 2011

Related U.S. Application Data
(63) Continuation-in-part of application No. PCT/US2010/026195, filed on Mar. 4, 2010.
(60) Provisional application No. 61/209,188, filed on Mar. 4, 2009.

(51) Int. Cl. A61M 37/00; A61B 18/20

(52) U.S. Cl. 604/20; 606/13; 606/9

(57) ABSTRACT

Disclosed herein are methods and apparatus for treating bladder cancer. Example apparatus include a laser or other source that generates visible or near-infrared radiation and a cytoscope to deliver and apply the radiation to a treatment site, such as the urothelial surface of the bladder. The radiation alters the permeability of at least one layer of the bladder wall, allowing more efficacious administration of chemotherapeutic or anticancer agent to the bladder lumen during or after application of the radiation. Permeability of the bladder wall may be altered by damaging suburothelial blood vessels, such as those of the lamina propria of the bladder wall, or by damaging the urothelium and suburothelial blood vessels of the bladder in a patient with bladder cancer. Damage to the urothelium induced by the radiation may be of a continuous or discontinuous nature. Treatment may also cause regression or destruction of (pre)malignant tissue in the urothelium and suburothelium.

### RELATED APPLICATION(S)

This application is a continuation-in-part of International Application No. PCT/US2010/026195, which designated the United States and was filed on Mar. 4, 2010, published in English, which claims the benefit of U.S. Provisional Application No. 61/209,188, filed on Mar. 4, 2009. The entire teachings of the above applications are incorporated herein by reference.

### BACKGROUND

### Basal Cell Carcinoma

Nonmelanoma skin cancer (NMSC), which includes basal cell carcinoma (BCC) and squamous cell carcinoma (SCC), is more common in the United States than all other types of cancers combined. Seventy-five to eighty percent of the estimated 1.7 million new cases of NMSC each year are BCC (Tierney E P, Hanke C W. J Drugs Dermatol 8; 914-922, 2009). NMSC is believed to develop over a period of years, subsequent to exposure of the skin to the ultraviolet component of sunlight, and generally occurs in people over the age of 50 years. The reported lifetime risk of BCC is 39% and 28% for men and women of European ethnicity, respectively (Robinson J K, Fisher S G. Arch Dermatol 136; 1318-1324, 2000). The incidence of NMSC is increasing in both older and younger populations. A study of a population in the vicinity of the Mayo Clinic in Minnesota has found that there has been a disproportionate increase in BCC in young women between the ages of 20 and 40 years, leading to concerns that this trend may lead to an exponential increase in the overall occurrence of these cancers as the population ages (Christenson L J, et al. JAMA 294; 681-690, 2005).

BCC is locally destructive but has a very low risk of metastasizing. For this reason, and because BCC is a skin lesion it might be assumed that it can be adequately addressed by surgical removal. Mohs micrographic surgery is the most tissue-sparing of BCC surgeries. It is also the most effective in removing all malignant cells associated with the lesion, and has a 5 year recurrence rate of only 1% for primary tumors. The cost of Mohs surgery depends on the number of stages or levels that are subjected to histological analysis for mapping of the tumor and the complexity of the repair needed for the resulting skin defect. Medicare costs for a relatively simple Mohs procedure are about $1500 when performed by a dermatologic surgeon in an office setting. Larger, deeper lesions or lesions in difficult anatomic locations can cost substantially more, as can Mohs procedures performed in other settings, or with other providers. Typically, Mohs surgery is reserved for BCC of the midface or ear, BCC of more aggressive histologic type (morpheaform and micronodular), and lesions that are large, of long duration, or otherwise at high risk of recurrence.

Surgical excision with immediate repair of the surgical defect is another standard treatment of BCC. Because of the difficulty in determining the subsurface spread and depth of the tumor and the competing need to preserve healthy, uninvolved skin tissue for optimal cosmesis and function, surgical excision has ten times the five year recurrence rates as Mohs surgery. Remarkably, approximately 15% of BCC treated with excision, with margins selected with the intention for total excision, have positive margins when examined subsequently by a pathologist (Hallock G G, Lutz D A. Plast Reconstr Surg 107; 942-947, 2001). A typical cost for surgical excision is about $1000, but with re-excision or Mohs for treatment of positive margins or tumor recurrence, the costs increase. The least costly methods of treating BCC are cryosurgery, and curettage and electrodessication (C&E). Cryosurgery is generally used to treat superficial BCC on the trunk or extremities. Because it is nonselectively destructive, scarring and hypopigmentation commonly result. As with C&E there is no opportunity to examine the margins, and so it is not a preferred treatment for BCC of aggressive subtypes or on locations associated with high risk of recurrence.

The role of medical therapy in treatment of NMSC including BCC is relatively limited. In recent years the topical immune response modifier imiquimod has been evaluated. (Love W E, et al. Arch Dermatol 145; 1431-1438. 2009). Imiquimod is currently approved by FDA only for treatment of superficial BCC less than 2 cm in diameter and only on the trunk, neck, or extremities. Therapy involves application five times a week for six weeks. When imiquimod has been used to treat nodular or micronodular BCC, it has been found that clearance of the tumor in the upper layers of the skin can mask residual deeper involvement and growth, possibly as the result of poor exposure of the deeper tumor to the topical drug (Sukai S A, et al. Derm Surg 35; 1831-1834, 2009).

Photodynamic therapy (PDT), which involves activation of a photosensitizing drug with light, has been studied for many years for treatment of BCC. Effective PDT requires that both drug and light fully penetrate the tumor. PDT using the photosensitizer aminolevulinic acid or its derivatives is approved for treatment of superficial BCC; however, this lesion type constitutes only about 20% of all BCC. A recent multi-site study reported short term results in nodular BCC; patients received two to four PDT sessions preceded by debridement and debulking of the tumor to facilitate penetration of the drug. Histologically verified complete response was 73% versus 27% for a placebo control group at 6 months. (Foley P, et al. Int J Dermatol 48; 1236-1245, 2009).

The present inventor first tested the use of the 585 nm pulsed dye laser (PDL) for BCC treatment (Beutner K R, Geisse J K, Alexander J, McMillan K, Lasers Surg Med Suppl 14; 22, 2002). The PDL was evaluated as a means of treating BCC by selective eradication of the blood supply on which the tumor cells depended. This study was followed by others (Allison K P, Kiernan M N, Waters R A, Clement R M, Lasers Med Scie 18; 125-6, 2003, Campolini P, Troiano M, Bonan P, Cannarozzo G, Lotti T. Dermatol Ther 21; 402-405, 2008, Shah S M, Konnikov N, Duncan L M, Tannous Z S, Lasers Surg Med 41; 417-422, 2009). These studies demonstrated the ability of PDL treatment to eradicate some BCC of different histologic types, and cosmetic results are excellent compared to the standard nonselectively destructive treatments. However several treatment sessions are typically required for successful eradication, and not all lesions respond completely. As a result, pulsed dye lasers or other vascular lesion lasers such as KTP are presently not viable alternatives to surgical excision for most patients. Improvement in treatment efficacy is required for vascular lasers to achieve their potential in treatment of skin cancer.

At present, with the number of BCC requiring treatment very high and increasing, there is a pressing need for a new treatment that is both highly effective and less costly than Mohs surgery or excision, that provides excellent cosmetic results, and that is preferably noninvasive.

### Bladder Cancer

In the United States, the incidence of new cases of bladder cancer in 2008 has been estimated at 68,810 (Jemal A, et al. CA Cancer J Clin 58; 71-96, 2008). The prevalence of bladder cancer is 450,000 to 619,000 (Botteman M F, et al. Pharmacoeconomics 21(18); 1315-1330, 2003). The remarkably high prevalence of the disease compared to its incidence is a consequence of its likelihood to recur after treatment. Patients typically live with bladder cancer for many years, during which time they may undergo frequent cystoscopic examinations, surgeries to resect recurrences, and repeated courses of chemo- or immuno-therapies. Consequently, bladder cancer has the highest per-patient total Medicare payments from diagnosis to death of any malignancy (Botteman M F, et al. Pharmacoeconomics 21(18); 1315-1330, 2003). From 1990-1 to 2004, the death rates for bladder cancer have been reduced by only 4.89 and 4.24% in males and females, respectively, rate reductions which are far lower than for many other types of cancer (Jemal A, et al. CA Cancer J Clin 58; 71-96, 2008).

Bladder cancer arises in the urothelium, or epithelial layer of the bladder wall. FIG. 1 is a schematic depiction of a bladder 1 and layers of the bladder wall 4. The bladder 1 is a pear-shaped hollow organ into which urine enters from ureters 2 through ureteral orifices 5, and exits through a urethra 3. The layers of the bladder wall 4 are, beginning with the bladder lumen, the urothelium 6, lamina propria 7, muscularis propria (muscle layer) 8, and adventia or serosa 9. A very thin basement membrane separates the urothelium from the lamina propria. Usage varies; the term mucosa sometimes includes both the urothelium and all or part of the lamina propria. Herein, with regards to the bladder, the term mucosa is equivalent to urothelium and submucosa is equivalent to lamina propria. Suburothelium as used herein is inclusive of all layers of the bladder wall excluding the urothelium, including but not limited to the lamina propria.

Bladder cancer is staged based on location of the tumors. Cancer that involves only the urothelium, or the urothelium and the lamina propria but not the muscularis propria, is referred to as superficial bladder cancer or nonmuscle invasive bladder cancer. Most bladder cancer patients present with superficial disease. Tumors are graded on the basis of histological evidence of aggressiveness; superficial bladder cancer may be of low to high grade, with higher grade tumors and carcinoma in situ being more aggressive.

Superficial bladder cancer treatment schemas usually begin with transurethral resection (TUR). Initial TUR can remove all visible superficial tumors for local control of disease, and to provide pathologic material for determination of tumor stage and grade for subsequent treatment planning TUR is accomplished using an electrosurgical cutting loop that cuts and coagulates tissue and that is introduced through a rigid cystoscope. TUR typically removes tissue down to the muscularis propria, and is performed in an operating room with the patient under general or spinal anesthesia.

If treated by TUR alone, the large majority of patients with superficial bladder cancer have tumor recurrence. Recurrences of bladder cancer after TUR can be attributed to (1) incomplete resection of the tumor, (2) a new tumor arising from dysplastic urothelium or carcinoma in situ at locations distant from the resection site, and (3) tumor seeding. To reduce the likelihood of recurrence and progression following TUR, courses of intravesical immunotherapy using bacillus Calmette-Guerin (BCG) or intravesical chemotherapy are used. Mitomycin C (MMC) is the most commonly used intravesical chemotherapeutic drug in the US. Both BCG and MMC are effective in reducing tumor recurrence, but BCG is superior and may also delay disease progression.

In the United States, BCG is typically used after TUR for high risk tumors, and as a first line therapy for carcinoma in situ which cannot be eradicated by TUR due to its diffuse nature. In the long term, however, over half of patients fail intravesical BCG therapy. Additional courses of BCG or maintenance regimens have been recommended but a significant number of patients are intolerant of BCG or have disease that is refractory to BCG therapy. Patients who fail a second course of BCG therapy have a very high risk of progression to muscle invasive disease.

In light of the significant side effect profile of BCG, courses of intravesical chemotherapy are frequently used instead of immunotherapy in treatment of intermediate risk variants of bladder cancer. Intravesical chemotherapy is also an option for treatment of patients with disease that is refractory to BCG or who have otherwise failed BCG therapy. Chemotherapeutic agents in use include MMC (dose 10-80 mg, Mutamycin®, Bristol-Meyers Squibb), thiotepa (dose 10-60 mg, Thioplex®, Amgen), doxorubicin (dose 10-80 mg, Adriamycin®, Bedford Laboratories), and epirubicin (dose 20-80 mg, Ellence®, Pfizer). Intravesical chemo- and immunotherapy is typically performed in a clinic or office with the patient under local anesthesia, and involves weekly instillations over a period of several weeks, with additional monthly maintenance treatments in the case of immunotherapy.

Intravesical chemotherapy as presently performed is limited in efficacy by suboptimal delivery of the drug to tissue. This suboptimal delivery may be caused by poor penetration of the drug through the urothelium, poor retention of the drug within suburothelial tissue, or both. The urothelium is relatively impenetrable to hydrophilic drugs. The most commonly used drug, MMC, is water soluble, disappears from the bladder wall rapidly after instillation, and has a concentration profile that decreases exponentially in suburothelial tissue (the half width ω_{1/2}, or thickness of tissue over which drug concentration declines by 50%, is only about 500 microns (Au J L-S, et al. J Control Rel 78; 81-95, 2002)). This half width is small compared to the thickness of the bladder wall, and as a result the exposure of tumor cells within the bladder wall to drug is highly inhomogeneous. Inhomogeneous and subtherapeutic concentrations of chemotherapeutic drugs in the bladder wall contribute to treatment failures, and disease recurrence and progression to muscle invasive disease.

Paclitaxel is a lipophilic drug that has shown high anticancer activity against bladder cancer cells. In aqueous form paclitaxel quickly penetrates the urothelium to a much higher extent than MMC. Lipophilic drugs have an advantage in being taken up by the urothelium, but present an inherent difficulty for intravesical use due to their very low aqueous solubility. The half width ω_{1/2} in bladder wall is 381 μm for paclitaxel in aqueous solution (Chen D, Song D, Wientjes M G, Au J L-S. Clin Cancer Res 9; 363-369, 2003). Gelatin nanoparticles (Lu Z, Yeh T-K, Tsai M, Au J L-S, Wientjes M G. Clin Cancer Res 10; 7677-7684, 2004) and bioadhesive monoolein delivery systems (Lee S-J, et al. Chemotherapy 51; 311-318, 2005) have been developed and tested in animal models, in attempts to overcome the difficulties of intravesical paclitaxel use. Notably, however, paclitaxel formulated according to those methods had reported ω_{1/2} values that are either decreased or statistically unchanged from that of paclitaxel in aqueous solution.

Attempts to use the penetration enhancer dimethyl sulfoxide (DMSO) to improve the pharmacokinetics of paclitaxel also resulted in decreased ω_{1/2} values, because capillary permeability was enhanced along with urothelial permeability (Chen D, Song D, Wientjes M G, Au J L-S. Clin Cancer Res 9; 363-369, 2003). Consequently, urothelial concentrations of paclitaxel can be varied by varying the urine concentration of the drug, or by changing the formulation of the drug, but the concentration of the drug as a function of depth below the urothelium in the bladder wall remains highly inhomogeneous with a ω_{1/2} value of less than 400 μm.

A number of other new drugs, including gemcitabine, docetaxel, eoquin, suramin, and γ-linolenic acid, have been evaluated in attempts to find intravesical drugs that have high anticancer activity against bladder tumors and that also can be delivered to the tissue layers of the bladder wall at therapeutic levels. Anticancer activity may be found with either lipophilic or hydrophilic drugs, and heterogeneity in bladder tumor response between patients to any one anticancer drug and the development of drug resistance makes it advantageous to have more than one drug available. Limitations on utility of anticancer drugs on the basis of permeability or retention in bladder wall is a significant disadvantage for the optimal treatment of bladder cancer patients.

It may be appreciated that improving the prognosis for bladder cancer and reducing the burden of its healthcare costs requires the development of improved treatments that reduce the risk of disease recurrence and progression, including the development of improved chemotherapies.

Thus, a need exists for improved methods and apparatus for treating cancer, particularly cancers of epithelial and mucosal origin, including bladder cancer and skin cancer.

### SUMMARY

The present invention is based on the recognition and collation of the following:

(1) electromagnetic radiation may be used to modify aspects of the urothelium, suburothelial tissue, or both, to increase and/or decrease tissue permeability, and alter and improve the pharmacokinetics of chemotherapeutic drugs in bladder tissue;

(2) electromagnetic radiation may be used to modify aspects of the suburothelial tissue to reduce the likelihood of growth of implanted or seeded tumor cells in the bladder wall;

(3) modification of the bladder tissue using electromagnetic radiation according to (1) or (2) has a concomitant direct effect on existing tumors, precancerous lesions, and/or lesional microvasculature;

(4) modification of the bladder tissue using electromagnetic radiation according to (1) or (2) increases the efficacy of hypoxia-activated chemotherapeutic drugs, and

(5) the effects of modification of the bladder wall, direct effect, increased efficacy and improved chemotherapy, singly or in combination, provide an advantageous method of treating superficial bladder cancer.

Methods and devices of the present invention can be used to induce photothermal injury in blood vessels and urothelium of a bladder having malignant and/or premalignant lesions, in such a way that chemotherapeutic drugs can be delivered with higher dosages to pathologic cells within the bladder wall. The present invention can also be used to induce photothermal injury in blood vessels and urothelium of a bladder having malignant and/or premalignant lesions, in such a way that chemotherapeutic drug is retained longer in tissues containing pathologic cells. The invention can also be used to modify tissue of the bladder using electromagnetic radiation, so that chemotherapeutic drugs can be delivered to tumor cells in the suburothelial tissue at a therapeutic dose.

Tissue modification is accomplished according to embodiments of the present invention by reducing the permeability of the suburothelial tissue to the drug, such that the drug is retained in the suburothelial tissue for a longer time, in higher quantities, or both. Tissue modification can also be accomplished by increasing the permeability of the urothelium, such that drug uptake from the urine, and therefore drug concentration in the urothelium or at the basement membrane located between the urothelium and the lamina propria are increased. Increasing drug concentration in the urothelium and/or at the basement membrane will increase the transport of drug by diffusion into the suburothelial tissue. Also, tissue modification can be accomplished by simultaneously increasing the permeability of the urothelium and decreasing the permeability of the suburothelial tissue to the drug.

Reduced permeability of the suburothelial tissue can be accomplished according to the invention by heating the blood vessels of said tissue by absorption of electromagnetic radiation to a temperature that coagulates the blood vessels or the contents of the blood vessels, or otherwise induces injury to suburothelial blood vessels, such that chemotherapeutic agents or drugs in the bladder wall are taken up and/or carried away by said blood vessels to a lesser extent than before said suburothelial tissue was irradiated. The characteristics of the electromagnetic radiation are such that the heating of suburothelial blood vessels is selective or partially selective, so that the blood vessels are heated to a higher temperature than the surrounding non-vascular suburothelial tissue. It is not necessary to photothermally injure all blood vessels in the bladder wall, and it may be preferable not to produce significant photothermal injury of the muscle layers of the bladder. In advantageous configurations, blood vessels of the lamina propria are injured to reduce blood flow in the lamina propria. In advantageous configurations, blood vessels of the superficial capillary plexus, mucosal plexus, and/or vessels interconnecting these two plexuses of the lamina propria, and/or the vessels connecting the mucosal plexus to the deeper layers of the lamina propria and/or muscularis propria, are photothermally injured to reduce blood flow in the lamina propria.

According to embodiments of the present invention, reduction in blood flow in the lamina propria by preferential absorption of radiation slows or impedes the transfer of drug molecules from the urothelium into the systemic circulation, in the absence of significant coagulative damage to nonvascular structures in the suburothelium that may be associated with side effects such as scarring or contracture. Coagulation of blood vessels and reduction in blood flow limits the uptake of drug by vasculature such that drug transport through the lamina propria may be limited to diffusion through extracellular space, thus increasing the time required for the drug to be eliminated from the suburothial tissue, thus increasing drug retention, and thus increasing exposure of tumor cells in the suburothelium to the drug. The percentage or amount of blood vessels damaged in the lamina propria can be varied by varying the energy density of radiation applied to the tissue, in order to vary the permeability of the lamina propria to the drug.

Electromagnetic radiation can be used to reduce the permeability of the suburothelium, or it can be used to simultaneously increase the permeability of the urothelium and reduce the permeability of the suburothelium. An increase in the permeability of the urothelium can be produced by inducing sufficient heating of the suburothelial blood vessels so that diffusion of said heat to the adjacent urothelium causes damage to said urothelium. Damage to urothelium, also referred to herein as urothelial layer, includes thermal injury to superficial, intermediate, and/or basal layer urothelial cells, loosening and/or separation of basal urothelial cells from the basement membrane, disruption of the connections and/or junctions between urothelial cells, disruption of desmosomes and/or hemidesmosomes in the urothelium, loss of urothelial cells from the urothelium, desquamation and/or damage to the mucin coating.

In advantageous configurations, urothelial layer damage is produced without substantial damage to the basement membrane at the junction of the urothelium and lamina propria. In advantageous configurations, urothelial layer damage is produced in a discontinuous manner, such that zones of damaged urothelium surrounded by zones of substantially less damaged urothelium are produced by the radiation. More advantageously, urothelial layer damage is produced in a discontinuous manner, such that zones of damaged urothelium surrounded by zones of substantially undamaged urothelial cells are produced by the radiation. Most advantageously, urothelial damage is produced in a discontinuous manner, such that zones of damaged urothelium, said damaged urothelium having a substantially intact basement membrane, are surrounded by zones of substantially undamaged urothelium or urothelial cells.

Chemotherapeutic drug instilled into a bladder after said bladder has been treated with electromagnetic radiation to reduce the permeability of the lamina propria may be retained longer in the lamina propria, compared to drug administered to an untreated bladder. Chemotherapeutic drug administered to the urothelial surface of a bladder wall after the bladder has been treated with electromagnetic radiation to increase the permeability of the urothelium may be present at a higher concentration within the urothelium, basement membrane, and/or junction of the urothlium with the lamina propria, compared to an untreated bladder. Said drug administered after the bladder has been treated to decrease lamina propria permeability and increase urothelial layer permeability may be present in the urothelium and/or lamina propria to a higher concentration, and is retained longer in the lamina propria, compared to an untreated bladder. Treatment of the bladder according to the present invention to reduce the permeability of the lamina propria, with or without increase in the permeability of the urothelium, increases the exposure of pathologic cells including tumor cells to chemotherapeutic drug.

Drug may be administered to the bladder according to the invention either during and after the irradiation of the bladder wall, or only after the irradiation of the bladder wall. Administration of the drug may be immediately after irradiation of the bladder wall, or there may be a delay of up to several days after irradiation, or until blood flow in the suburothelial tissue is substantially returned to normal by repair of damaged blood vessels or growth of new vessels.

The drug may be any chemotherapeutic agent that acts upon any abnormal, diseased, transformed, dysplastic, cancerous, or pre-cancerous cells, tissue component, or tissue. An advantage of the methods and apparatus of the present invention is that they are adaptable to any agent that can be applied to cells or tissue, including therapeutic, diagnostic, and anesthetic agents. The drug may be lipophilic or hydrophilic. The drug can be a single agent, or combination of agents. Furthermore, the drug may be in any formulation, vehicle or carrier and may be used with penetration enhancers.

An aspect of the present invention is that use of electromagnetic radiation for improved delivery of drugs to tissue can have a concomitant effect on cancerous, precancerous, dysplastic, or abnormal cells, or the supporting vasculature of those cells. Selective or partially selective damage to suburothelial blood vessels will damage blood vessels supplying tumors or tumor cells in the lamina propria and/or urothelium. Because the urothelium does not contain blood vessels, urothelial cells are supplied by blood vessels of the underlying lamina propria. Selective or partially selective damage to lamina propria blood vessels injures cells including premalignant and malignant cells in the urothelium or lamina propria by removing the source of nutrients from said cells and by inducing hypoxia. Furthermore, diffusion of heat from damaged suburothelial blood vessels to the urothelial layer may cause direct thermal injury to urothelium, and malignant and/or premalignant cells in the urothelium.

Another aspect of the present invention is that use of electromagnetic radiation for improved delivery of drugs to tissue can have the concomitant effect of preventing growth of tumors as a result of tumor cell seeding or implantation in the bladder. Radiation that produces selective or partially selective damage to suburothelial blood vessels reduces the ability of individual tumor cells within the urothelium or seeded cells adhering to the urothelium to establish a blood supply and develop into a tumor. Irradiation of the bladder wall to produce selective or partially selective damage to suburothelial blood vessels can be performed according to the present invention to reduce the ability of seeded tumor cells to grow into tumors, with or without administration of a chemotherapeutic agent.

In another aspect of the invention, the use of electromagnetic radiation to damage suburothelial blood vessels for improved pharmacokinetics of chemotherapeutic drugs, or for prevention of tumor cell seeding, can be used to increase the cytotoxicity and efficacy of certain chemotherapeutic drugs. These drugs include the commonly used alkylating agent mitomycin C, and are of a class termed bioreductive drugs. Bioreductive drugs can be activated to kill tumor cells in bladder tissue that is made hypoxic or relatively hypoxic by the methods and devices of the present invention. Thus, in a bladder wall treated so that suburothelial blood vessels are damaged to reduce permeability of the lamina propria, a chemotherapeutic drug may be made more active against tumor cells and such tumor cells will also be exposed to higher levels of the drug.

Yet another aspect of the invention is that the urethra and/or ureters may be treated, solely or in addition to the bladder, for modification of urethral and/or ureteral walls, direct effect on pathologic cells, hypoxia-induced drug activation, and/or improved chemotherapy.

Apparatus of the invention may include a source of electromagnetic radiation, such as a laser or other light source that emits radiation in the visible or near infrared regions of the spectrum. Some embodiments may include a pulsed visible or near-infrared laser or light source adapted to be connected to an optical fiber that can be inserted into the working channel of a cystoscope, such that the distal end of the fiber can be positioned at or near the urothelial surface of the bladder to deliver radiation to injure the urothelium and/or suburothelial blood vessels prior to or during instillation of a chemotherapeutic drug. According to preferred embodiments of the invention, the radiation source is a pulsed dye laser, a pulsed neodymium YAG or KTP laser, a flashlamp-pumped alexandrite laser, or a pulsed diode laser.

Example laser radiation sources of the present invention can be configured to operate according to the methods of the invention, and also in ablative, incisional, and/or coagulative modes. Example apparatus may be used for additional applications, such as for use as a multi-purpose surgical tool in urology.

Methods and devices of the invention provide a treatment for bladder cancer that reduces recurrence rates, delays progression of disease, or both. The invention also provides for a reduction in morbidity associated with treatment of cancer. The present invention also provides a treatment for cancer that can be performed under local anesthesia in the doctor's office or clinic. Furthermore, the present invention provides a treatment for cancer that reduces health care costs.

The present invention can be adapted for the treatment of malignant and premalignant conditions other than bladder cancer, and for the treatment of noncancerous conditions.

### BRIEF DESCRIPTION OF THE DRAWINGS

FIG. 1 is a schematic depiction of a urinary bladder 1 showing ureters 2, urethra 3, bladder wall 4, and ureteral orifices 5. The layers of the bladder wall 4 are indicated in the inset: urothelium 6, lamina propria 7, muscularis propria 8, and adventitia/serosa 9.

FIG. 2 is a schematic depiction of the components of epithelial and subepithelial tissue as related to the diffusion and uptake of drug molecules.

FIGS. 3A, 3B, and 3C are results of mathematical model calculations of the effect of a 585 nm pulsed dye laser on normal skin tissue at fluence 4 J/cm², 9 J/cm², and 14 J/cm², respectively. FIG. 3D is the result for skin tissue with a BCC tumor treated at 14 J/cm².

FIG. 4A is a schematic drawing of an applicator of the invention.

FIGS. 4B, 4C, and 4D are schematic drawings of light-absorbing elements of an applicator of the invention.

FIGS. 5A and 5B are results of a model calculation of the effect of a 585 nm pulsed dye laser with an applicator having light-absorbing elements.

FIG. 6 is a schematic drawing of the placement of treatment areas over the area of a skin tumor, using the applicator of FIG. 4A.

FIG. 7 is a schematic drawing of an applicator of the invention, where the ablation element is smaller than the irradiated area.

FIG. 8 is a schematic drawing of the placement of a treatment area over the area of a skin tumor, using the applicator of FIG. 7.

FIGS. 9A, 9B, and 9C are results of a model calculation of the effect of a 1064 nm Nd:YAG laser on skin tissue and skin tissue with a basal cell carcinoma.

FIG. 10 is the result for a model calculation of the effect of a 532 nm KTP laser on skin tissue with a basal cell carcinoma.

FIGS. 11A and 11B are schematic depictions of ablation elements, with a thin window disposed between the light-absorbing elements and tissue, and with tissue-penetration projections, respectively.

FIG. 12 is a schematic depiction of the vascular architecture of the bladder wall 4. The avascular urothelium 6 lies over the lamina propria 7, which in turn overlies the muscularis propria 8. In the lamina propria is the superficial capillary plexus 10, perpendicular vessels 11 connecting the superficial capillary plexus and the vessels of the mucosal plexus 12. The mucosal plexus is connected to perpendicular vessels 13 descending into the deeper layers of the lamina propria and muscularis propria.

FIG. 13 shows the results of a mathematical model calculation of the effect of a 585 nm pulsed dye laser on a bladder filled with saline. Temperature at the end of a laser pulse is plotted as a function of depth in the bladder wall, for several different laser fluences, at the center of the irradiated spot. The position of the junction of the urothelium and lamina propria at 150 μm is indicated by the solid vertical line.

FIGS. 14A-14D are contour plots of results of mathematical model calculations of the effect of a 585 nm pulsed dye laser on a bladder filled with saline, for four different representative laser fluences. The contour plots are of temperature as a function of depth in the bladder wall (z axis), and distance from the center of the beam across the surface of the bladder (x axis). The model includes blood vessels representative of the vascular architecture of the lamina propria.

FIG. 15 shows the results of a model calculation of the effect of a 755 nm pulsed alexandrite laser on a bladder filled with saline. Temperature at the end of a laser pulse is plotted as a function of depth in the bladder wall, for laser fluences between 30 and 60 J/cm² and a 3 ms duration pulse, and for a fluence of J/cm² and a 300 μs pulse, at the center of the irradiated spot.

FIGS. 16A and 16B are contour plots that show results of model calculations of the effect of a 755 nm pulsed alexandrite laser on a bladder filled with saline, for two different representative laser fluences.

FIG. 17 shows the results of a model calculation of the effect of a 532 nm pulsed KTP laser on a bladder filled with saline. Temperature at the end of a laser pulse is plotted as a function of depth in the bladder wall, for laser fluences between 10 and 20 J/cm² and a 15 ms duration pulse.

FIGS. 18A and 18B are contour plots that show results of model calculations of the effect of a 532 nm pulsed KTP laser on a bladder filled with saline, for two different representative laser fluences.

FIG. 19 shows the results of a model calculation of the effect of a 1064 nm pulsed neodymium YAG laser on a bladder filled with saline.

FIGS. 20A and 20B are contour plots that show results of model calculations of the effect of a 1064 nm pulsed neodymium YAG laser on a bladder filled with saline, for two different representative laser fluences.

FIGS. 21A and 21B are contour plots of the temperature at the basement membrane for irradiation of bladder tissue with a KTP laser and optical fiber delivery system producing a Gaussian or a collimated energy distribution on the urothelial surface.

FIG. 22 shows the results of a model calculation of the effect of a 1064 nm pulsed neodymium YAG laser on a bladder filled with saline, with radiation delivered using a contact tip probe.

FIG. 23 shows the results of model calculations of the effect of an 800 nm pulsed diode laser on a bladder filled with saline.

FIG. 24 shows the concentration of paclitaxel as a function of depth in the bladder wall, comparing bladder with varying amounts of suburethelial vascular damage (0 to 90%).

FIG. 25 shows the concentration of paclitaxel as a function of depth in the bladder wall, for various amounts of suburothelial vascular damage (0 to 90%) in the presence and absence of urothelial damage.

FIG. 26 shows the concentration of paclitaxel as a function of depth in the bladder wall, when administered with DMSO, or when administered without DMSO but with urothelial damage and various amounts of suburothelial vascular damage (0 to 90%).

FIG. 27 shows the concentration of mitomycin C as a function of depth in the bladder wall, for no urothelial damage or partial urothelial damage, and varying amounts of suburothelial vascular damage (0 to 90%).

FIGS. 28A and 28B show schematic views of a laser 20 and a flexible cytoscope 30, respectively, of an embodiment of the present invention. The embodiment also includes a laser optical fiber 25 positioned with distal end adjacent to a wall of the bladder 1.

FIGS. 29(a)-29(d) show concentration profiles for MMC in dermis, at application times of 5 min (a), 30 min (b), 2 hrs (c), and 8 hour (d), as a function of vascular damage from 0% to 75%.

FIG. 30 shows time dependent MMC concentrations at depth in tissue, with (75%) and without (0%) vascular injury.

FIG. 31 shows surface exposure to MMC, for subsurface (z=1.5 mm) IC₉₀ exposures, with (75%) and without (0%) vascular injury.

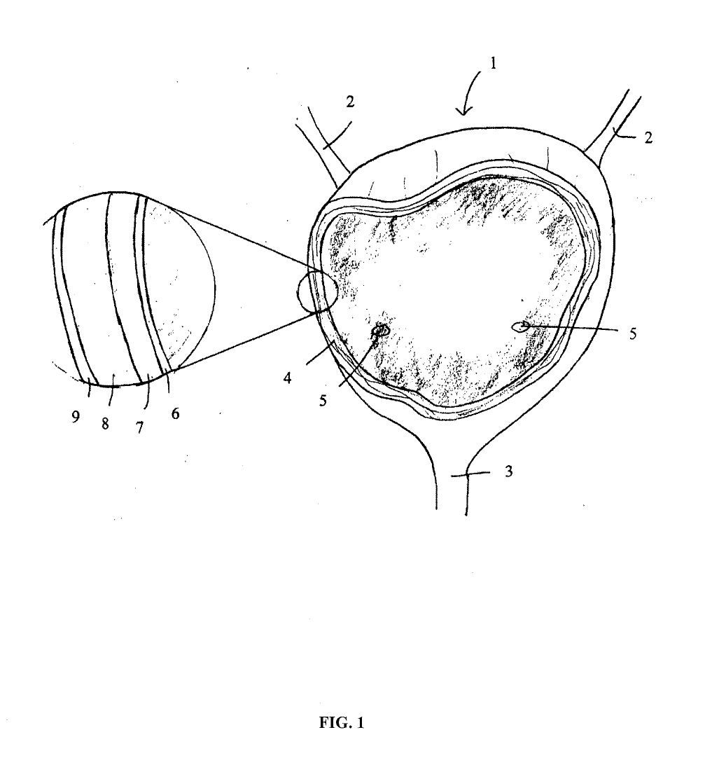

### DETAILED DESCRIPTION

The methods and devices of the present invention address the problem of treating cancer or precancer, particularly cancer of epithelial origin, which includes skin tumors, precancerous skin lesions, proliferative skin lesions, bladder cancer, and Barrett's esophagus, among others.

Embodiments of the present invention include an apparatus for modifying a tissue for administration to the tissue of a therapeutic agent, which may be or include a chemotherapeutic drug, an anticancer drug, and/or a bioreductive drug. Example apparatus include a source configured to generate radiation selected to reduce permeability within the tissue to the therapeutic agent and an applicator coupled to the source and configured to apply radiation from the source to the treatment site. Example tissue may be or may include tissue in a bladder, a ureter, and/or skin.

In some embodiments, the source is further configured to generate radiation selected to cause photothermal injury to blood vessels of a subsurface layer and to a surface layer of the tissue. The photothermal injury increases exposure of the subsurface layer to the therapeutic agent. Suitable sources include, but are not limited to: a pulsed laser, a continuous-wave laser, and a scanned laser. Suitable lasers include KTP lasers, dye lasers, neodymium YAG lasers, alexandrite lasers, semiconductor diode lasers, and fiber lasers. Alternatively, the source may include a continuous-wave incoherent source and/or a pulsed incoherent source. The source can be capable of generating radiation at a wavelength of between about 400 nm and about 1100 nm; a pulse duration of between about 300 ns and about 100 ms; and/or an energy density of between about 3 J/cm² and about 80 J/cm².

Example applicators are configured to apply radiation to an epithelial layer and an upper subepithelial layer and to avoid or prevent damage to a lower subepithelial layer. The epithelial layer may include urothelium, the upper subepithelial layer may include lamina propria, and the deeper subepithelial layer may include muscularis propria. The applicator may also include an ablation element that has a tissue-contacting surface and at least one light-absorbing (chromophore) element embedded in, attached to, or coating the tissue-contacting surface of the ablation element. Example light-absorbing (chromophore) elements include carbon, pyrolytic carbon, iron oxide, and other pigments.

Other embodiments include tissue-treatment apparatus that comprises a light source, an applicator coupled to the light source and configured to deliver light emitted by the light source to a surface of the tissue via an ablation element, which has a front surface and a back surface, and at least light-absorbing element embedded in, attached to, or disposed over the front surface or the back surface of the ablation element. The tissue may be or include skin tissue, and the light source may be configured to emit light comprising at least one wavelength preferentially absorbed by blood.

The front surface can be a tissue-contacting surface configured to contact the tissue surface directly, the light-absorbing elements can be configured to contact the tissue surface directly (and independently of the front surface), or both the front surface and the light-absorbing elements can be configured to contact the tissue surface directly. For example, the light-absorbing elements may protrude from the front surface of the ablation element, allowing the front surface to stand off from the surface of the tissue—that is, the light-absorbing elements provide a gap between the front surface and the tissue surface.

Some embodiments of the tissue-treatment apparatus may include a cooling element configured to cool the tissue, the ablation element, or both the tissue and the ablation element. For instance, in embodiments with protruding light-absorbing elements, air or other cooling fluid may be passed through the gap between the front surface and the tissue surface to cool the tissue surface and the front surface.

In example tissue-treatment apparatus, the ablation element may be or may include a window and/or a lens. The tissue-contacting surface of the ablation element may have a diameter of between about 3 mm and about 20 mm, and light impinging on the back surface of the ablation element may be substantially uniform in energy density.

Embodiments of the tissue-treatment apparatus may also have one or more light-absorbing elements that include at least one chromophore each. In addition, the light-absorbing element(s) may be selected from the group consisting of carbon, pyrolytic carbon, iron oxide, and other pigments; the light-absorbing element(s) may also include layers, films, paint, and/or other coatings. The light-absorbing element(s) may also have a spatially varying thickness, e.g., as in a wedged or sculpted layer or film. Alternatively, the light-absorbing element(s) can include plural light-absorbing elements arranged in an array disposed over or parallel to the front surface and/or the back surface of the ablation element. Embodiments include both nonuniform (i.e., aperiodic) and uniform (i.e., periodic) arrays.

Further embodiments are methods of modifying a tissue of a mammalian body that include generating radiation; conveying the radiation to a treatment site of the tissue; and reducing permeability of the tissue to a therapeutic agent by applying the radiation to the treatment site. Application of the radiation causes thermal injury to blood vessels at the treatment site. Methods may include applying the therapeutic agent to the tissue during or after application of the radiation such that thermal injury increases exposure of the tissue, which may be or include tissue in a bladder, a ureter, and skin, to the therapeutic agent, which may include a chemotherapeutic drug, anticancer drug, and/or a bioreductive drug.

The tissue may be a multilayered tissue that includes an epithelial layer and an upper subepithelial layer. Reducing permeability of the tissue can include causing photothermal injury to blood vessels of the upper subepithelial layer. In these embodiments, the photothermal injury can be sufficient to reduce blood flow in the upper subepithelial layer such that exposure of the upper subepithelial layer to the therapeutic agent is increased during application of the therapeutic agent. If the multilayered tissue also includes a deeper subepithelial layer, reducing permeability of the tissue may also include avoiding or preventing damage to the deeper subepithelial layer. In some embodiments, the epithelial layer includes urothelium, the upper subepithelial layer includes lamina propria, and the deeper subepithelial layer includes muscularis propria.

Reducing permeability of the tissue can include, but is not limited to: thermally injuring epithelial cells in the epithelial layer; loosening connections between adjacent epithelial cells in the epithelial layer; loosening connections between basal epithelial cells and a basement membrane of the tissue; damaging a mucin layer of an epithelium of the tissue; detaching epithelial cells; and inhibiting growth of tumor cells seeded on or in a surface of the treatment site. Application of radiation may leave the basement membrane substantially intact.

Reducing permeability of the tissue may also include producing a discontinous injury to the epithelial layer of multiple zones of thermally injured epithelial cells. At least one of the thermally injured zones can have an area of less than about 1 cm², and may have an area of less than about 0.1 cm². Thermal injury can be produced by radiation generated at a wavelength of between about 400 nm and about 1100 nm, a pulse duration of between about 300 ns and about 100 ms, and/or an energy density of between about 3 J/cm² and about 80 J/cm².

Yet further embodiments include methods of inhibiting tumor growth in tissue, comprising generating radiation and conveying the radiation to a treatment site of the tissue. Growth of tumor cells seeded on or in a surface of the tissue is inhibited by applying the radiation to the treatment site to cause thermal injury to blood vessels at the treatment site. Alternative embodiment methods include methods of treating a multilayered tissue, with an upper layer and a lower, of a mammalian body. In these alternative embodiments, radiation is generated and conveyed to a treatment site of the tissue. Then blood flow in the lower layer is reduced by causing photothermal injury to blood vessels of the lower layer through application of the radiation to the tissue. This reduction in blood flow prevents growth of living tumor cells implanted on or attached to the upper layer.

Still further embodiments are methods for treatment of a tissue, such as skin, that include placing a distal surface of an applicator in contact with a tissue surface. The distal surface of the applicator includes one or more light-absorbing (chromophore) elements, and the applicator is configured to direct light from a light source via the distal surface to the tissue surface. A pulse of light is generated with the light source and absorbed by the one or more light-absorbing (chromophore) elements and by blood vessels in the tissue under the distal surface. The applicator is removed from the tissue surface, and a therapeutic substance is applied to tissue surface.

Absorption of the pulse of light by the light-absorbing (chromophore) elements causes at least a portion of the tissue surface to be ablated or removed. Ablation or removal of the portion of the tissue surface can be sufficient to substantially increase the permeability of the tissue surface layer to the therapeutic substance. Similarly, absorption of the pulse of light by the blood vessels causes at least a portion of the blood vessels under the tissue surface to be coagulated. Coagulation of the portion of the blood vessels under the tissue surface can be sufficient to substantially reduce the permeability of the tissue surrounding the blood vessels to the therapeutic substance.

Yet another embodiment is a method for treatment of tissue that comprises placing a light delivery element adjacent to a tissue surface of the tissue. A pulse of light is initiated/generated from a light source and conveyed from the light source to the tissue surface via the light delivery element. Blood vessels in the tissue absorb at least some of the pulse of light such that at least a portion of the tissue surface is damaged, and such that a portion of the blood vessels under the tissue surface are coagulated. A therapeutic substance is applied to surface of the treatment area.

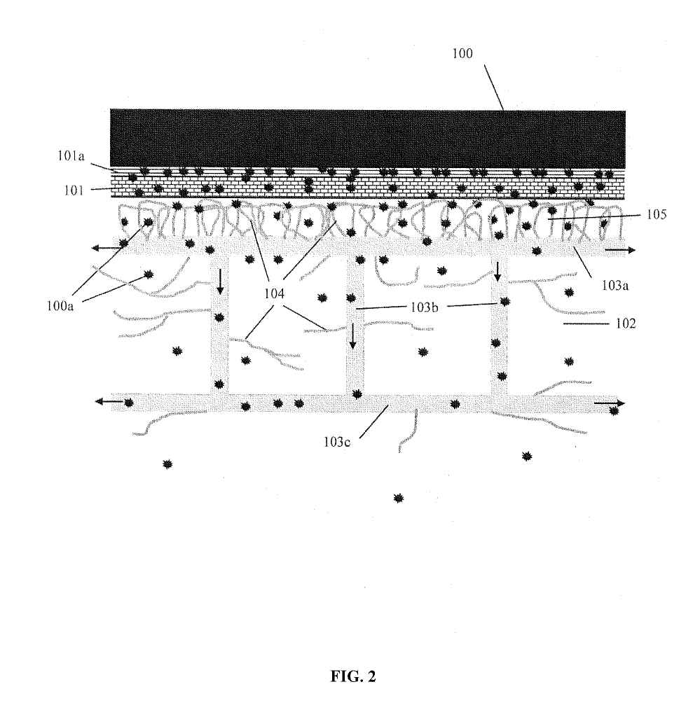

The schematic drawing of FIG. 2 illustrates the problem that is solved by the present invention. A layer of drug in solution (100) is in contact with tissue comprising an epithelium or epithelial layer (101) and a subepithelial layer (102). The epithelial layer (101) may have an outermost barrier layer (101a). For example, if the tissue is skin, the epithelial layer, or epidermis, has an overlying stratum corneum. The stratum corneum may be considered as part of the epidermis.

Tumor cells may reside in the epithelial and/or subepithelial layers of tissue. Solute drug molecules must penetrate the epithelium to reach the subepithelial layer, and any tumor cells it may comprise. Because the epithelium does not contain blood vessels, drug molecules (100a) travel through the epithelium by a process of diffusion, from the higher concentration of the solution layer (100). Consequently, the drug concentration is linear with distance through the epithelium. Especially if there is an effective barrier layer such as stratum corneum, the concentration gradient may be very steep.

Once drug molecules pass the junction between epithelial and subepithelial layers, they continue to diffuse towards the deeper subepithelium. However, subepithelial tissue is richly vascularized, and drug molecules may be taken up by blood vessels to be carried into the systemic blood supply. Typically, subepithelial tissue has at least 2 horizontal networks of larger microvessels (upper horizontal plexus 103a, lower horizontal plexus 103c) as well as interconnecting ascending and descending arterioles and venules (103b). The first and by far the most numerous blood vessels that are encountered by drug molecules diffusing through the subepithelial tissue are capillary vessels (104), including the many capillary loops between the upper horizontal plexus (103a) and the epithelium, often referred to as the superficial capillary plexus (105). Other capillaries (104) are found at lower density throughout the subepithelial layer (102). Capillary vessels are the smallest microvessels, and are also believed to be the only permeable microvessels in tissue. Drug molecules crossing the capillary vessel walls are transported to the larger microvessels of the upper horizontal plexus, descending vessels, and lower horizontal plexus. Therefore it is well established (see e.g. Kretsos K, Kasting G B, Skin Pharmacol Physiol 18; 55-74, 2005) that capillaries, specifically the most superficial capillaries, are responsible the extraction of drug from the subepithelial layer before it reaches the deeper subepithelial tissue and vessels therein, and, ultimately, systemic circulation. Within the subepithelial layer, drug concentration falls exponentially with distance from the epithelium.

It is recognized that there are anatomic differences between different types of tissue, for example between skin and bladder wall. However, it is generally true that there are two main impediments to achieving a high or therapeutic concentration of a drug within tissue where a tumor or tumor cells resides: (1) the epithelial barrier which limits the amount of drug that can enter the epithelial and subepithelial tissue, and (2) the blood vessels of the subepithelium that contribute to rapid loss of the drug within that tissue layer. The present invention addresses both of these impediments. As will be described below, the method and devices of the present invention allow a higher concentration of drug to be achieved in the tissue, for a longer period of time, thereby increasing the exposure of any tumor cells in the tissue to a therapeutic drug dosage.

Specifically, the present invention provides a means for modifying tissue permeability so that when a drug is topically applied, a more effective exposure of tissue, tumor, and tumor cells to the drug may be achieved.

### Basal Cell Carcinoma

The present invention is first described in detail for the case of treatment of BCC, where the tumor resides in skin tissue.

According to the present invention, the tissue blood vessels that contribute to rapid loss of drug are selectively injured, incapacitated, or made less permeable by irradiation with light that is preferentially absorbed by blood.

The use of lasers or other light sources to selectively or preferentially coagulate or otherwise injure blood vessels is well known in the art as a means of treating vascular lesions, for example portwine stain (PWS) birthmarks and telangiectasias. Light sources that have been used clinically for photothermal vascular targeting include 585-600 nm PDLs, 1064 nm Nd:YAG, 532 nm KTP, 755 nm alexandrite, and various near-infrared diode lasers, among others, as well as incoherent intense pulsed light sources (IPLs). Pulse durations in the millisecond time domain (1-100 ms) are most typical, although the 585 nm PDL has a 0.5 ms pulsewidth. These light sources, to greater or lesser degree, are capable of selectively coagulating the abnormal microvessels constituting vascular lesions. For example, PWS lesions are made up of ecstatic vessels within the dermis, having diameters of 30 to 150 μm or larger for older, thicker lesions, and may respond well to treatment with pulses of 0.5 ms or longer. However, it has long been recognized that these light sources in clinical use are not capable of coagulating the smallest blood vessels in PWS lesions in infants, or in normal skin. Histologic studies have shown conclusively that blood vessels of 25 μm or smaller diameter in the skin do not respond even to the short 0.5 ms pulse of the 585 nm PDL. This finding is in agreement with theory of selective photothermolysis, as the thermal relaxation time of capillary vessels (external diameter 10-12 μm) is shorter than 0.5 ms, meaning that heat will diffuse out of the vessels during the laser pulse, limiting the ability of the pulse to increase the temperature within the vessel. The inability of the PDL or other vascular lesion lasers to coagulate capillaries is also responsible for the observation that such devices can eradicate abnormal, lesional vessels without also causing ischemic necrosis of the skin. Shorter pulse duration laser (e.g. prototype 577 nm PDLs operating with 350 ns pulses) have been tested and found to produce mechanical damage to blood vessels, including capillaries, as a consequence of confining heat to erythrocytes which rise to temperatures over 100° C. and explosively rupture. However, for reasons discussed below, according to the present invention, there are advantages to using light sources with pulses of about 0.5 ms or longer.

Thus, according to the present invention, photothermal injury of blood vessels of the skin is performed to prevent those vessels from serving as pathways for the elimination of drug molecules from the surrounding subepithelial tissue, although the vessels that are responsible for drug uptake (capillaries) are too small to be photothermally injured by the lasers and IPLs currently in clinical use for photothermal treatment of cutaneous vascular lesions.

A novel concept underlying the approach of the present invention is that rather than directly targeting the capillaries responsible for drug uptake, the larger venules and arterioles of the subepithelial tissue can be coagulated or otherwise thermally injured, thus sealing off the routes of blood flow between the capillaries and the systemic circulation. Injury of a portion of the larger microvessels will reduce the amount of drug removed by the vasculature, and will affect pharmacokinetics.

It is noted that the degree of vascular injury required to reduce the amount of drug removed via the subepithelial vasculature may range from coagulation of the vessel along with an amount of perivascular tissue, to partial coagulation or temporary occlusion of the vessel, to a more subtle injury that has an effect on the permeability of vessel walls. Also, the degree of vascular injury that is most useful clinically may depend on the anatomic site of treatment and the drug that is being used. An aspect of the present invention is that the parameters of the laser or light source (fluence, wavelength, spot size, and pulse duration) may be varied to achieve the degree of vascular injury that is most effective and beneficial for a particular case.

In an aspect of the present invention, in order for the treatment to have a more advantageous effect on drug pharmacokinetics, the photothermal injury of blood vessels is selective or preferential, such that the surrounding nonvascular tissue of the subepithelial layer is substantially uninjured by heat, substantially uncoagulated, or otherwise left substantially permeable so that drug molecules may continue to diffuse into said surrounding tissue from the overlying epithelium. It may be particularly advantageous to avoid denaturation of the cellular and extracellular matrix proteins of the subepithelial tissue (dermis in the case of skin) to the extent that the tissue is heat-fixed and substantially impervious to drug diffusion.

On the basis of these concepts, a detailed description is made herein using model calculations, in which the interaction of light with tissue and the transfer of heat within the tissue are determined. These model calculations simulate the effect of light on skin tissue, and are an approximation to the results of the actual treatment of a patient's skin with a laser or other light source, according to the present invention. The laser-tissue interaction is calculated with a Monte Carlo algorithm to simulate the paths of photons through the tissue. The calculation volume is rectangular with 2 cm by 2 cm surface area and a depth of 1 cm. The resolution of the Monte Carlo calculation in each direction is 50 μm. One million photons are included in each calculation, which assumes a flat-top laser beam incident on the skin tissue surface. Heat transfer calculations are done numerically by a finite-difference method.

In these model calculations, tissue is represented by the following layers, beginning with the topmost or most superficial layer: (1) epidermis, assumed to be a layer 100 μm in thickness, (2) dermis, 2.6 mm in thickness, and (3) subcutaneous tissue, with infinite thickness. The thicknesses used are exemplary; skin tissue is typically thinner on the face, especially around the eyes, and thickest on the back. Also in the model, the microvasculature of the dermis is represented by two horizontal 50 μm diameter blood vessels located 0.5 and 2.5 mm under the surface, representing the upper and lower vascular plexuses, respectively, and a series of vertical 50 μm diameter blood vessels spaced 1 mm apart, connecting the two horizontal vessels. The vertical vessels represent the ascending and descending vessels of the dermis. In some model calculations, a spherical tumor nodule is also included. Not explicitly include are capillary sized vessels, which are less than 10 microns in diameter and too small to be modeled, however the absorption coefficient for dermis used in the model reflects the blood component of capillaries. Also not explicitly included in the model is the stratum corneum of the epidermis, which is only about 20 μm thick.

Calculations performed herein focus on normal skin and nodular type basal cell carcinoma. Nodular BCC tumors are most common and, due to their tendency to grow deeply, are potentially more difficult to treat by light-based means than thinner superficial BCC. Normal skin is modeled to determine the effect of the treatment on skin surrounding the tumor and the tumor margin zone, which in an aspect of the invention is a part of the treatment. Choice of nodular BCC for modeling provides a stringent test of the present invention.

The Monte Carlo model uses well known optical properties for skin components from the literature, listed in Table I below, for 585 nm, 532 nm, and 1064 nm, wavelengths that are readily available from PDLs, KTP lasers, and Nd:YAG lasers, respectively.

TABLE I
Optical constants for skin components.

| | 585 nm | | 532 nm | | 1064 nm | |
|---|---|---|---|---|---|---|
| blood | | | | | | |
| μₐ | 177 | cm⁻¹ | 225 | cm⁻¹ | 4.3 | cm⁻¹ |
| μₛ | 764 | cm⁻¹ | 692 | cm⁻¹ | 654 | cm⁻¹ |
| g | 0.95 | | 0.96 | | 0.97 | |
| dermis | | | | | | |
| μₐ | 1.89 | cm⁻¹ | 2.69 | cm⁻¹ | 0.51 | cm⁻¹ |
| μₛ | 166 | cm⁻¹ | 195 | cm⁻¹ | 89 | cm⁻¹ |
| g | 0.8 | | 0.8 | | 0.8 | |
| epidermis | | | | | | |
| μₛ | 3.36 | cm⁻¹ | 5.32 | cm⁻¹ | 0.246 | cm⁻¹ |
| μₛ | 266 | cm⁻¹ | 319 | cm⁻¹ | 146 | cm⁻¹ |
| g | .8 | | .8 | | 0.8 | |
| basal cell carcinoma | | | | | | |
| μₐ | 1.65 | cm⁻¹ | 2.50 | cm⁻¹ | 0.21 | cm⁻¹ |
| μₛ | 130 | cm⁻¹ | 153 | cm⁻¹ | 58 | cm⁻¹ |
| g | .8 | | 0.8 | | 0.8 | |
| subcutaneous tissue | | | | | | |
| μₐ | 2.54 | cm⁻¹ | 4.03 | cm⁻¹ | 0.734 | cm⁻¹ |
| μₛ | 224 | cm⁻¹ | 262 | cm⁻¹ | 117 | cm⁻¹ |
| g | 0.8 | | 0.8 | | 0.8 | |

It may be noted that for BCC tissue (nodular subtype), both the absorption and scattering coefficients (μₐ and μₛ, respectively) are lower than the corresponding coefficients for the surrounding dermis. This may be understood in terms of characteristics of nodular BCC, in which tumor cells grow in rounded masses within the dermis. The nodular BCC tissue is predominantly cellular and therefore less scattering than the extracellular dermal matrix with collagen fibrils. Nodular BCC have been shown to lack microvessels within the tumor interior, although at their boundary there is a proliferation of blood vessels, including relatively large caliber blood vessels. The paucity of blood vessels within the tumor nodule accounts for the lower absorption coefficient for the tumor.

Calculations are first done for normal skin tissue, to demonstrate that laser treatment can preferentially target the relatively large microvessels of the normal dermis, to thermally injury those vessels so that that routes of drug molecule travel from the capillaries to the systemic circulation are eliminated. In these and all other model calculations herein, maximum temperatures are limited to 100° C., because the heat transfer calculation does not take into account phase change.

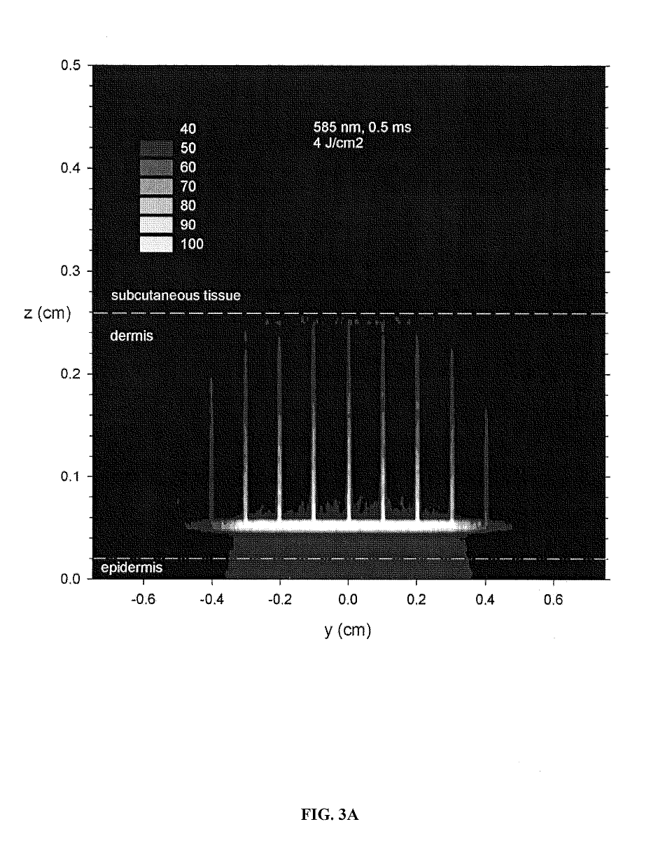

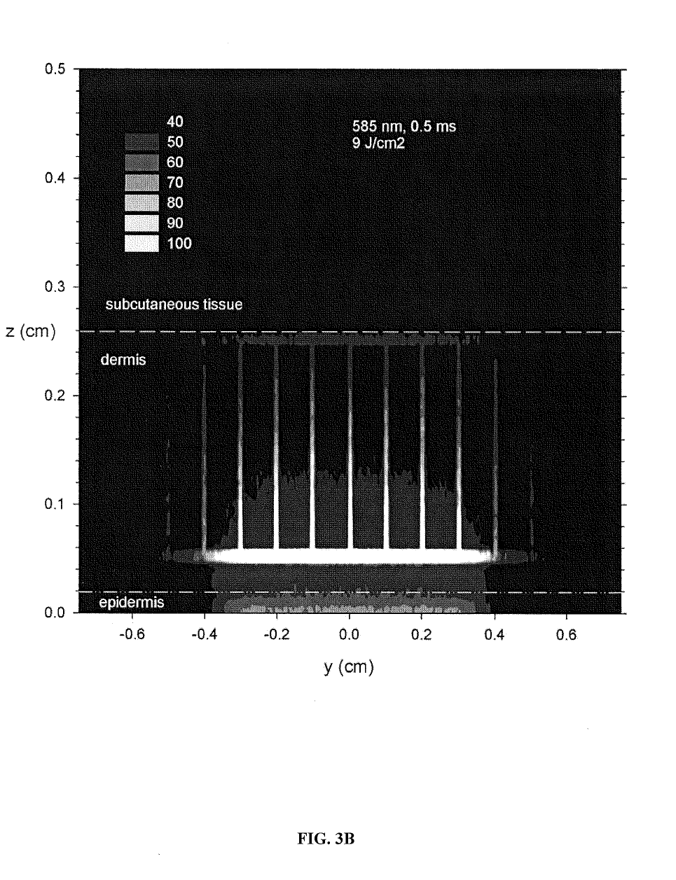

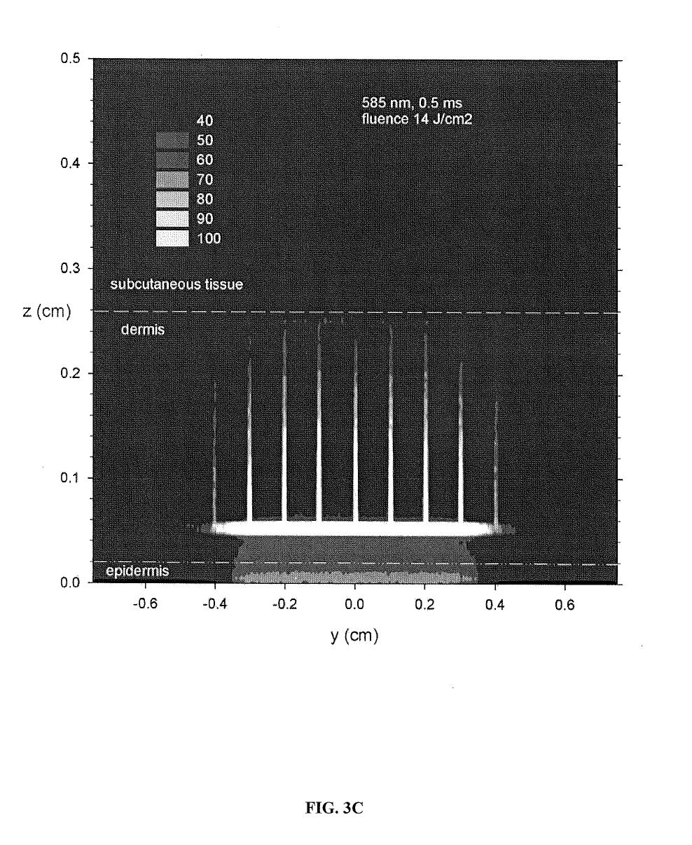

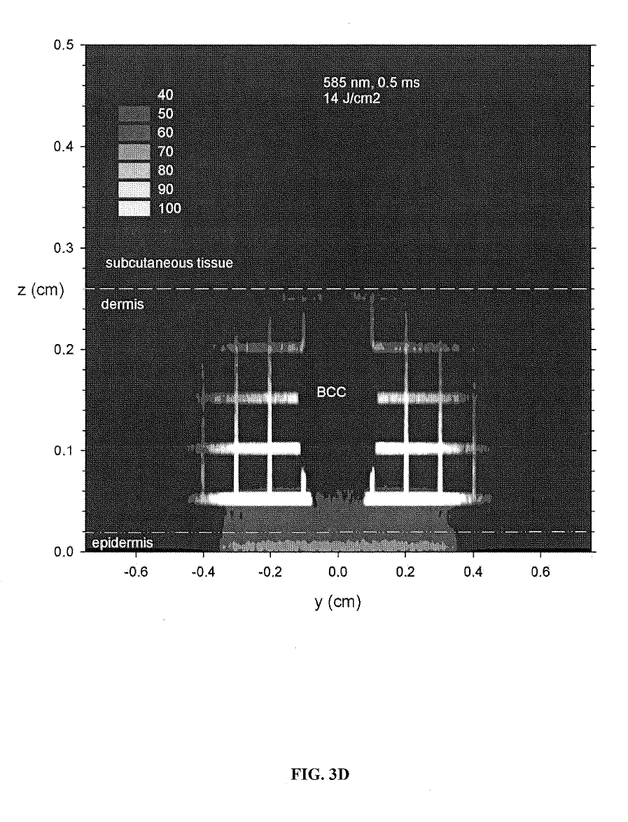

In FIG. 3A, the results are shown, in the form of a contour plot of temperature at the end of the laser pulse, as a function of location under the skin surface. In this and the other contour plots provided herein, y is a dimension parallel to the tissue surface, z is the depth perpendicular to the surface, and the origin of the coordinate system is the center of the laser beam on the tissue. In this particular calculation, the model assumes pulses with 0.5 ms pulse duration and fluence (energy density on the skin surface) of 4 J/cm². The pulse has a diameter on the skin surface of 7 mm, and a top-hat, evenly distributed beam profile. The skin is exposed to room temperature air during the non-contact pulse. As is known to those skilled in the art, these laser parameters from a 585 nm PDL typically produce microvascular injury within the dermis, as evidenced by purpura or bruising. As can be seen in FIG. 3A, the model calculation accurately predicts vascular coagulation across the horizontal vessel of the upper plexus, as well as upper portions of the vertical vessels, in agreement with clinically observed purpura. Over a 0.5 ms time period, a temperature of about 70° C. or higher may be expected to cause thermal injury to tissue. In FIG. 3A, it can be seen that the 4 J/cm² laser pulse produces temperatures of at least 70° C. in the vertical blood vessels down to a depth of 1.5 mm near the center of the beam, and 1.0 mm near the edges. Also in FIG. 3A, and in agreement with clinical observation, the PDL at this fluence substantially avoids temperatures corresponding to thermal injury to the overlying epidermis.

These findings indicate that these laser parameters produce a deeper injury to vessels in normal skin, than in PWS lesions, where vascular coagulation is known to be limited to depths of well under 1 mm.

In FIG. 3B, the same model is used with pulses of higher fluence (9 J/cm²). This fluence is seen to produce increased heating of the dermal microvasculature, with blood vessel coagulation expected down to about 1.5 mm at the edges and 1.9 mm near the center. Again, the areas of dermis surrounding the blood vessel are substantially unheated. The epidermis shows heating in the 60 to 70° C. range, which may be expected to cause some thermal injury or partial coagulative necrosis within this layer. Again, this calculation is in agreement with clinical experience, and supports the accuracy of the mathematical model developed herein. Clinically, in the treatment of skin lesions such as PWS birthmarks, these laser parameters would require skin cooling to protect and preserve the epidermis for optimal cosmetic outcome. Skin cooling may take the form of a cryogen spray, a chilled contact element such as a window or lens, a cooling fluid, cold air applied to the skin surface before, during, and/or after a laser pulse, or any other means known in the art.

In FIG. 3C, a similar model, but with a sapphire contact window for tissue surface cooling, is used to evaluate the effect of 14 J/cm² pulses from the 585 nm, 0.5 ms laser. The sapphire window is maintained at 4° C., and placed in contact with the skin 50 ms before the laser pulse. Here, the maximum depth of coagulation of the representative 50 μm diameter vertical vessels of the dermis is slightly over 2 mm from the tissue surface. The epidermis is also injured, with at least partial coagulation over most of the irradiated area.

Selective vascular coagulation of 2 mm depth corresponds to the thickness of skin on areas of the body other than the back (where it may be thicker), or around the eyes and other facial locations (where it may be thinner). Consequently, these calculations indicate that the depth of penetration of 585 nm light in skin is sufficient for effective vascular targeting in skin tissue over most of the body where BCC appear. This finding is further tested in the model calculation of FIG. 3D, where a spherical tumor module with diameter 2.4 mm is included. Also included in the tumor model are additional 50 μm diameter blood vessels running horizontal to the skin surface, representing feeder vessels produced by the tumor. FIG. 3d shows that the tumor feeder vessels down to approximately 2 mm from the skin surface will be partially or completely coagulated. The tumor itself is dark in the contour plot, because of its low absorption coefficient relative to dermis.

According to the present invention, if a drug is applied to the skin surface immediately before or immediately after the laser pulses of FIG. 3A-D, the extent of dermal vascular damage to the vessels of the upper plexus and vertical vessels would be expected to prevent uptake of drug by dermal vasculature. As noted above, capillary vessels, which are not part of the mathematical model, are important in actual living skin tissue in taking up and carrying away drug molecules traveling through dermis. Particularly important are the capillaries of the superficial capillary plexus directly underneath the epidermis. Pulse durations on the order of a millisecond or tens of milliseconds, typical of vascular lesions lasers, are too long to preferentially injury capillaries. However, according to the present invention, it is unnecessary to damage capillaries, if the larger arterioles and venules of the upper horizontal plexus are damaged. Damage to the latter constitutes “downstream” damage to limit drug molecules from being carried into the systemic circulation from substantially undamaged capillaries under the epidermis. Thus, the targets of photothermal injury, according to the present invention, for treatment of skin and skin tumors, are the somewhat larger arterioles and venules (approximately 15 to 100 microns in diameter) of the upper and lower horizontal plexuses and the ascending and descending vessels, as well as the feeder or supply vessels of a tumor.

Thus, it is apparent that according to the present invention, the vascular architecture of a region of the skin can be targeted in a manner that changes the permeability of said skin region to a drug. Specifically, arterioles and venules normally present in the skin region can be preferentially thermally injured to reduce uptake of the drug by the blood vessels, including capillary blood vessels. This novel effect can be achieved using vascular lasers, such as the 585 nm PDL, developed and commercialized for treatment of abnormal vasculature, such as portwine stains, telangiectasias, and the like.

This vascular-specific injury does not address the other impediment to drug exposure in skin, namely the barrier effect of epidermis, particularly the stratum corneum of the epidermis. In the absence of skin cooling, the heating of the epidermis shown in FIG. 3B, for example, would be expected to lead to blistering and/or coagulative necrosis, over all or part of the irradiated epidermis, but such an effect may take hours to fully develop and may not immediately disrupt the epidermis and its barrier function. With time after coagulation or other irreversible thermal injury, the epidermis may separate and slough, but the immediate post-irradiation effect will be a damaged epidermis that may be loosened at the basement membrane but still intact. Furthermore, the amount of such epidermal damage is difficult to predict as it depends strongly on the amount of melanin in the basal layer of the epidermis, which is highly variable, even among light phototype people of European ethnicity most prone to BCC. Yet furthermore, epidermal injury will be greatest at the basal layer, and less at the relatively unpigmented stratum corneum, where the barrier function of the skin is highest. As a result, skin treated with the parameters of FIG. 3B (without cooling) may have an unpredictable and unenhanced penetration by topical drugs applied to the skin surface at or about the time of irradiation.

Therefore, the present invention includes a means of producing precise and localized ablations of the stratum corneum and/or epidermis, for improved penetration of a topically applied drug into the dermis.

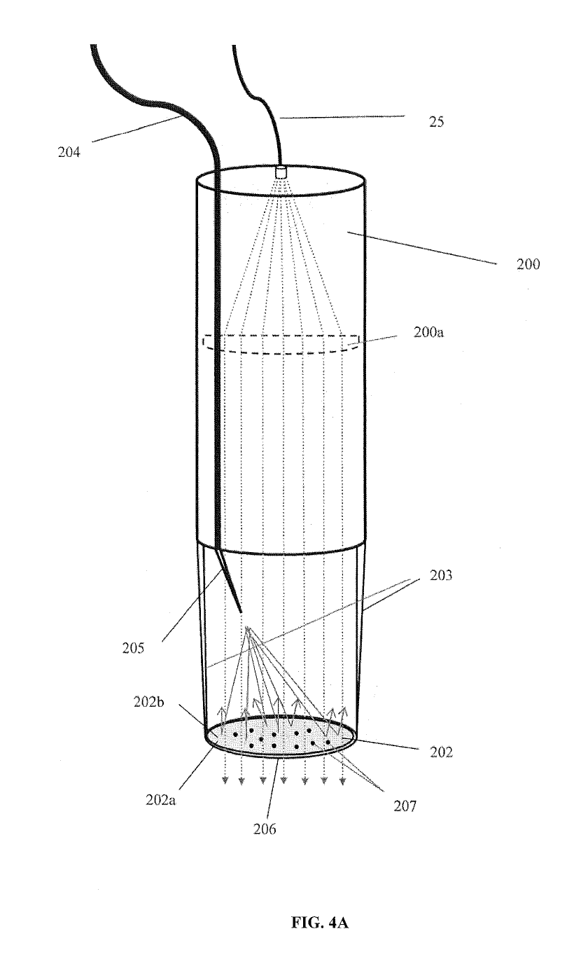

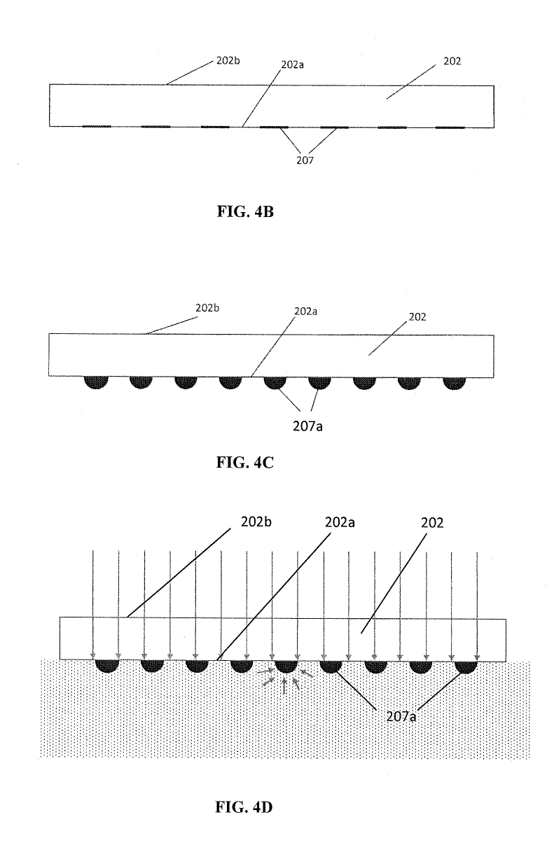

FIG. 4A is a schematic depiction of an embodiment of the device of the invention, which comprises a laser handpiece or light applicator (200). A laser fiber (25) transmits light from a laser source through one or more lenses (200a) or optics of the applicator and onto an ablation element (202). The ablation element (202) is designed to contact the skin surface, and in some embodiments comprises a light transmissive material, such as glass, optical plastic, sapphire or the like. The ablation element (202) may be secured in a ring (206) and held at a distance from the laser fiber (25) by stand-off segments (203). The ablation element has a front surface (202a) and a back surface (202b) upon which light from the light source is incident. The light source, fiber, and applicator optics may be configured to produce incident light on 202a having a uniform energy distribution (beam profile), or may be configured to produce a nonuniform beam profile on 202a. The beam profile is approximately the same size as the ablation element, or smaller. The back surface (202b) may have an antireflection coating, or it may be uncoated. The ablation element (202) comprises light-absorbing elements (207), which may include chromophores, which are in contact with or in near proximity to the skin surface when the applicator is applied to the skin surface. Also shown in FIG. 4A are skin cooling elements, comprising a cooling line (204) and a nozzle (205) for emission of a cooling fluid (gas, liquid, or cryogen) that in some implementations of the present invention, may be used to reduce the temperature of the ablation element (202), the tissue, or both. In the embodiment depicted in FIG. 4A, cooling fluid impinges on the back surface (202b) of the window.

In some embodiments of the invention, at least one light-absorbing element (207) is placed in contact with or on the topmost layer of the skin, and/or near but not in contact with said topmost layer prior to irradiation. In an advantageous configuration, the light-absorbing elements may be in the form of a coating applied to the front side of an ablation element. The ablation element may be a substantially transparent optical element, such as a window or lens, comprising light-absorbing elements. Where it does not impinge on light-absorbing elements, light passes through the ablation element. Light-absorbing elements may be discrete, or discontinuous, for example arranged as a group, pattern, or array on a surface or within the ablation element. Light-absorbing elements may also be continuous, with a varying density or thickness over or within an ablation element, thereby determining the passage of light through the ablation element. In more advantageous implementations of the present invention, discrete light-absorbing elements are configured so that the size and or depths of the ablations of the stratum corneum and/or epidermis can be controlled by the size and density of the elements as well as by the thickness of light absorbing material and choice of light absorbing material making up the light-absorbing elements. Said thickness of the light absorbing material may be constant or spatially variable. In this manner, the permeability of the epithelium can be increased in an advantageously precise and predictable manner, by selection of the design parameters of the light-absorbing element and the laser parameters. In a very advantageous aspect of the present invention, the same light pulse that heats and injures the dermal microvasculature to reduce vascular uptake of drug, thereby reducing permeability of the dermal layer, also heats the light-absorbing element or elements in contact with skin surface, thereby ablating a portion or portions of the stratum corneum and/or epidermis, and thereby concomitantly increasing the permeability of the epidermis, in a simultaneous action.

FIG. 4B is a schematic depiction of an embodiment of an ablation element (202) with light-absorbing elements (207) coated or painted areas on the window surface, in a side view. For example, the elements (207) may be screen printed paints containing a pigment or chromophore that absorbs light of a wavelength or wavelengths emitted by the light source. Or, the light-absorbing elements may be produced by a light absorbing coating using a masking process, according to other techniques well known in the art. In the embodiment of FIG. 4B, the at least one light-absorbing elements comes in contact with the tissue surface when the applicator is held against the skin.

It is recognized that the light-absorbing elements will reduce the amount of light that is transmitted into the tissue. For example, if the irradiated spot size is 7 mm in diameter, and the light absorbing elements are circular with a 250 μm diameter and arranged in a cubic pattern with 1 mm edge length, there will be a total of 37 elements with a total area of 5% of the irradiated spot size. If the light absorbing elements have a 500 μm diameter, with the same cubic arrangement, the elements will comprise a 19% of the total area. To compensate for the light loss at depth in the tissue due to the light-absorbing elements at or near the tissue surface, the energy of the light source may be increased.

FIGS. 4C and 4D are schematic depictions of another advantageous configuration of the light-absorbing elements. Light-absorbing elements in the shape of blunt projections (207a) are designed to be pressed onto the skin surface, so that said skin surface substantially conforms to the surface of the projections and the surrounding front surface of the ablation element. In this embodiment, the skin tissue is contacted by the light-absorbing elements but is not substantially penetrated or broken by said elements. By using elements that are shaped as blunt projections from the window, the effective area of the elements adjacent to the tissue surface is increased, for a given area of said elements on the window surface 202a. For example, if the elements have a hemispherical shape with radius 500 μm, the area of tissue in contact with those elements will be about 57% greater than tissue in contact with flat elements of the same radius. Hence, the size of the ablations may be increased without an increase in light loss. Furthermore, the blunt projections will be irradiated by backscattered light within the skin tissue over their entire surface, as shown in FIG. 4D, increasing the ablative effect without an increase in fluence. The light-absorbing elements of FIGS. 4C and 4D may comprise coating or paints applied to the surface of blunt projections of an ablation element, or the blunt projections may be made of a shaped light-absorbing element attached to or embedded in an ablation element.

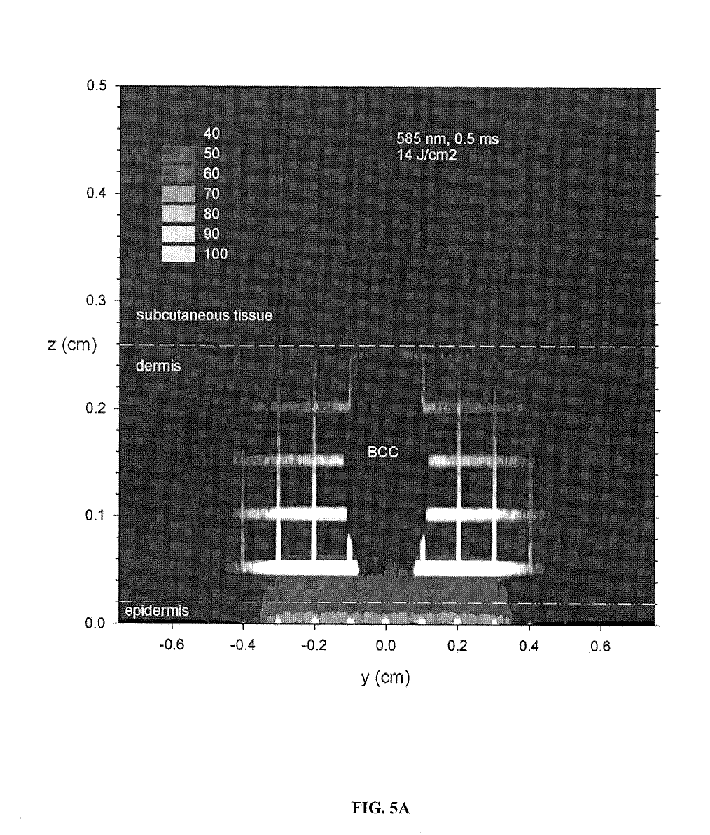

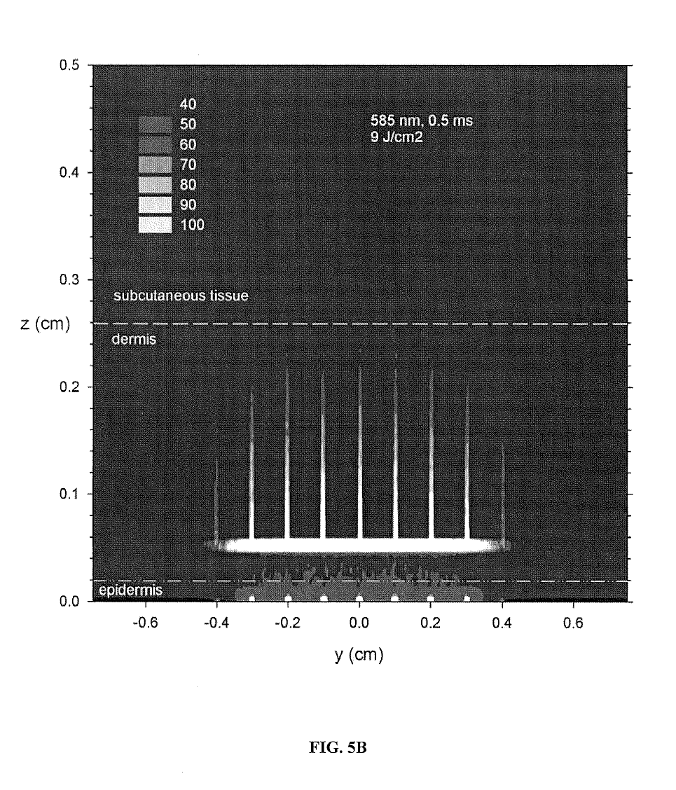

FIG. 5A shows the results of calculations for the model of FIG. 3D, that is, for a 585 nm, 0.5 ms PDL pulse on skin with a BCC nodule. The only change to the model is the addition of light-absorbing elements in the form of circular light absorbing coated areas of 100 μm diameter, separated at 1 mm intervals, on the sapphire window surface. The light-absorbing elements are 10 μm in thickness. In this model, the ablation element of the invention is the sapphire window with coated areas. The sapphire window is cooled to 4° C. and applied to the skin immediately before the laser pulse. Ablations in the skin with depth of about 50 μm, or about half the epidermis including the stratum corneum, are created, (Herein, it is assumed that tissue that reaches temperatures of 100° C. or higher at the end of a light pulse are vaporized, immediately removed, or ablated.) These ablations created by the light pulse may be expected to significantly increase permeability of the epidermis, while the same light pulse simultaneously causes substantial damage to the vasculature surrounding the BCC tumor. In thin skin, the ablations found in the results of FIG. 5A may be sufficient to penetrate the full thickness of epidermis. In that case, the residual thin coagulative zone between about 70 but less than 100° C. around those epidermal ablations will prevent substantial bleeding, such that an advantageously bloodless treatment is provided.

In FIG. 5B, the cooled ablation element of FIG. 5A is added to the normal skin model of FIG. 3B. Here, the surface cooling allows the epidermis to remain substantially uncoagulated or uninjured, between ablation zones corresponding to the location of light-absorbing elements on the ablation element.

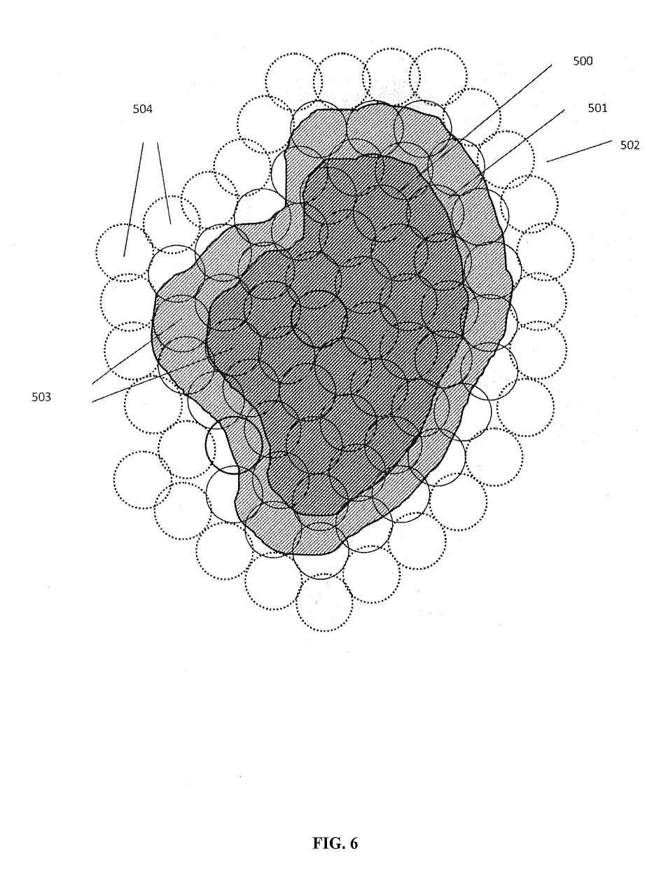

FIG. 6 depicts the use of an applicator of the invention, for example the applicator depicted in FIG. 4A, in treatment of a skin lesion, for example a BCC with large surface area. As is standard in BCC treatment, the physician would first determine on the basis of biopsy and/or clinical examination, the extent of the lesion in the skin. This lesional area (500) may be assumed to have tumor cells in or under the surface of the tissue. Surrounding the lesional area is a peri-lesional margin (501) that may have tumor cells, although these cells are not clinically detectable. This peri-lesional margin area may be the same tissue that is excised as a safety margin when treating a BCC by surgical excision. The safety margin may typically be 4 to 5 mm for BCC, although with more aggressive histologic types or recurrences, the safety margin may be increased. Surrounding the peri-lesional margin area is presumed normal tissue (502). As noted in the Background, presumed normal tissue beyond the typical safety margin of a BCC may comprise tumor cells. An important advantage of the present invention is the ability to treat normal skin surrounding a skin tumor, for reduced recurrence and improved efficacy, without substantial damage to said normal skin.

According to an implementation of the invention, the laser applicator of FIG. 4A would be used to treat all three areas (500, 501, and 502). First, the lesional area (500) is treated with laser fluence sufficient to cause substantial coagulation of blood vessels in the dermis, substantial coagulation of epidermis, and at least one ablation zone in the epidermis. (Epidermal coagulative necrosis of the lesional area is advantageous because tumor originates with the epidermis. Lesional and peri-lesional epidermis is completely excised as part of standard excisional treatment of BCC.) In one example, the lesional area may be treated with laser pulses using the parameters and ablation element described for the model of FIG. 5A. These higher energy pulses (503) may be intended to produce substantial vascular damage at depths within a tumor. Overlapping higher energy pulses (503) are used as may be required to cover all the lesional area with substantially no gaps. Then, the peri-lesional area (501) is treated. When treating the peri-lesional area, it may be acceptable to reduce the amount of overlapping of the higher energy pulses (503), thereby treating less aggressively. Then, the laser fluence is reduced to a value that still produces substantial coagulation of blood vessels in the dermis, and at least one ablation zone in the epidermis, but avoids substantial coagulation of the epidermis. In one example, the lesional area may be treated with laser pulses using the parameters and ablation element described in the model of FIG. 5B. The lower fluence pulses (504) are applied to the normal skin, in an overlapping or non-overlapping manner.

In this manner, the present invention allows skin tissue containing a BCC to be treated to increase permeability of the epidermis over the known site of the lesion and an area around said site, while simultaneously reducing the permeability of dermis and dermis containing tumor cells. Because ablation zones are produced in the normal skin epidermis around the lesion but remaining non-ablated epidermis is substantially uncoagulated, said normal skin epidermis heals quickly and with optimal cosmesis. Tissue in all areas—lesional, peri-lesional, and surrounding normal tissue—is exposed to topical therapeutic drug applied to the tissue surface and penetrating through the epidermal ablations. In this manner, more tumor cells may be exposed to the effects of treatment of the present invention by the combined action of the drug and the laser, than is the case with standard excisional surgery which does not treat the apparently normal surrounding tissue.

Consequently, for a tumor of a given size, the present invention allows a larger area of skin tissue to be treated while at the same time being more sparing of normal tissue. The important advantages of this aspect of the invention are greater efficacy in tumor eradication combined with improved cosmesis and function. Also, the treatment is advantageously rapid and bloodless.

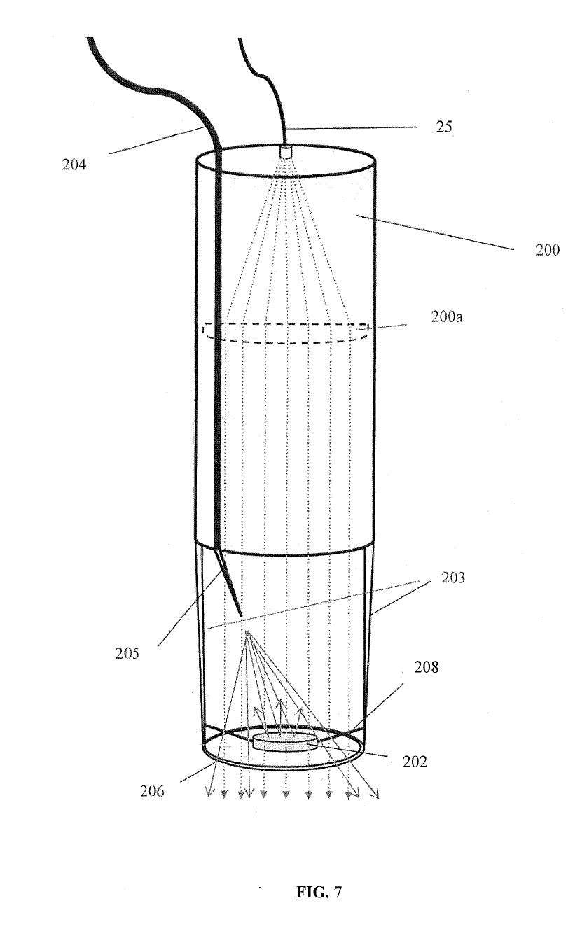

Another embodiment of the present invention is shown in FIG. 7. In FIG. 7, the applicator is similar to that of FIG. 4A, with the exception of the ablation element (202), which in this embodiment is smaller than the laser beam, so that a portion of the beam exiting the applicator impinges directly on the skin. The ablation (202) may be connected to the handpiece by supports (208). In some embodiments, a cooling element directs coolant towards the ring and the tissue contacting window.

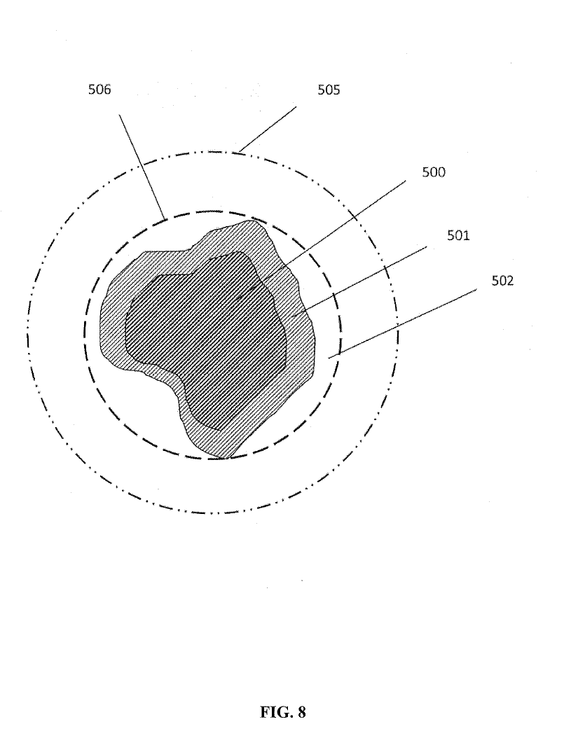

FIG. 8 depicts the use of the applicator of FIG. 7 to treat an area of skin comprising a BCC. In this instance, the BCC is relatively small. The applicator is placed against the skin surface so that the ablation element covers the lesional area (500) and peri-lesional or safety margin area (501). A normal skin area (502) is located within the ring of the applicator, but outside the area covered by the ablation element. The area covered by the ablation element is outlined by the dashed circle (506) and the irradiated area is outlined by the dashed-dotted line (505). Consequently, the normal skin area is cooled directly by the cooling elements, such that coagulative injury to the epidermis can be avoided. The ablation element covering the lesional and peri-lesional areas is cooled directly by the cooling element. Depending on the thickness and choice of material for the ablation element, as well as the choice of cooling parameters, the lesional and peri-lesional areas under the ablation element may be conductively cooled by said ablation element. For example, to treat a 3 mm diameter BCC, an applicator may be configured with a 10 mm diameter ablation element, and an irradiated spot size of 18 mm. The cooling elements may provide a flow of −25° C. air directed towards the ablation element and surrounding skin. With a fluence of 9 J/cm² and pulse duration of 0.5 ms, 585 nm PDL pulses may be expected to produce damage to deep subsurface vessels of the normal skin beyond the ablation element, but no substantial epidermal damage with the coolant flow. Underneath the ablation element, the lesional and peri-lesional tissue will have both deep subsurface vascular injury, and ablation zones in the epidermis. If the ablation element is, for example, a thin sapphire window of 1 mm thickness, and the coolant flow is initiated before the laser pulse, the conductive cooling to the skin may prevent thermal injury to epidermis between the ablation zones. However, if the ablation element is selected to be thicker, for example several millimeters, and/or made in part or whole of a material with a lower heat transfer coefficient, for example quartz or optical grade laser resistant plastic, conductive cooling of the skin under the ablation element may be minimal and the skin may be thermally damaged or coagulated between the ablation zones.

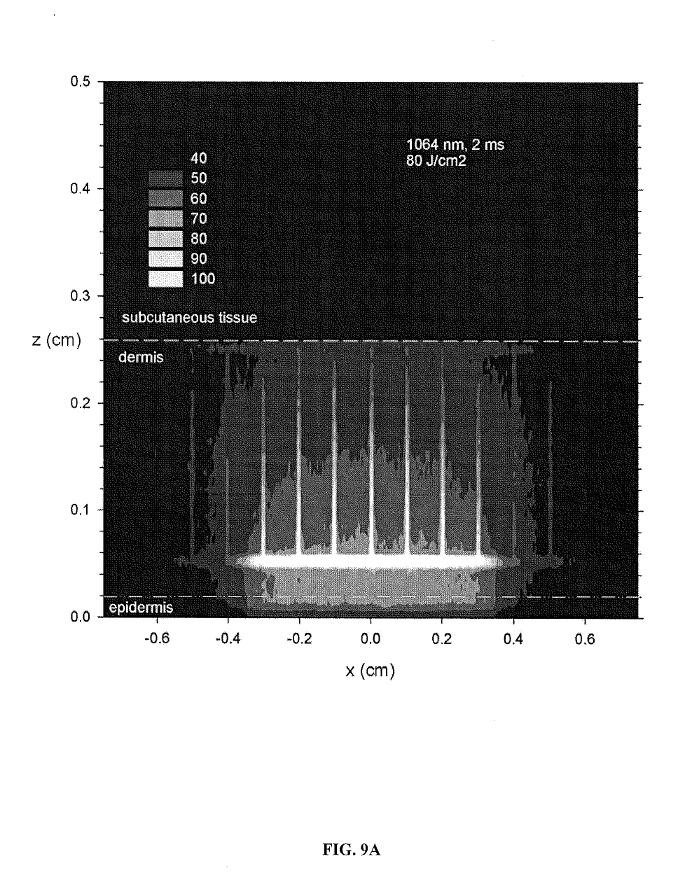

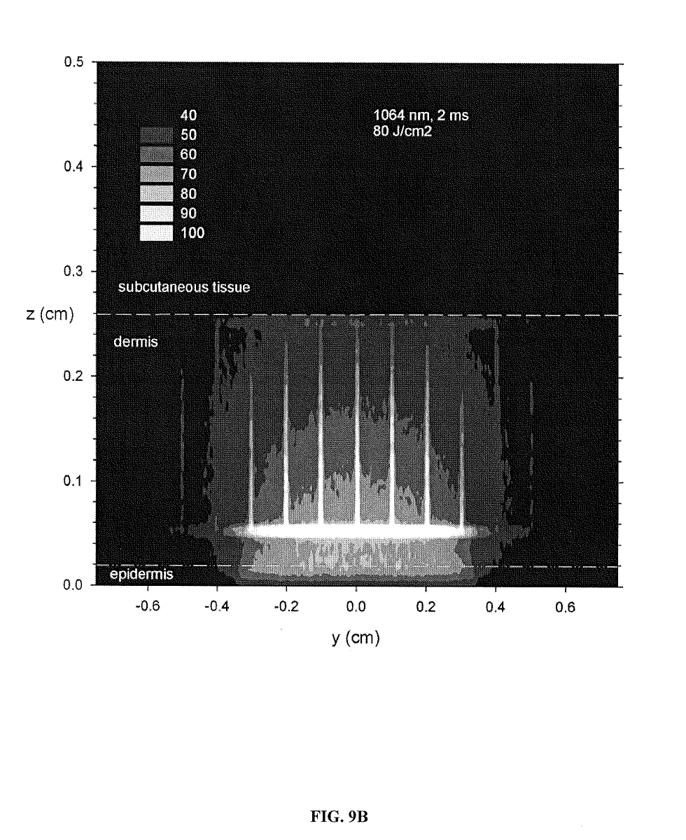

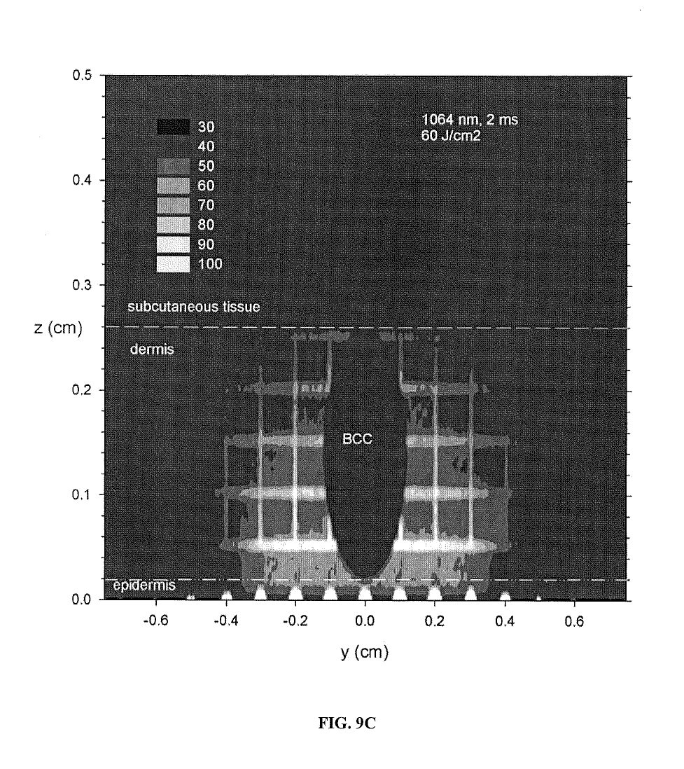

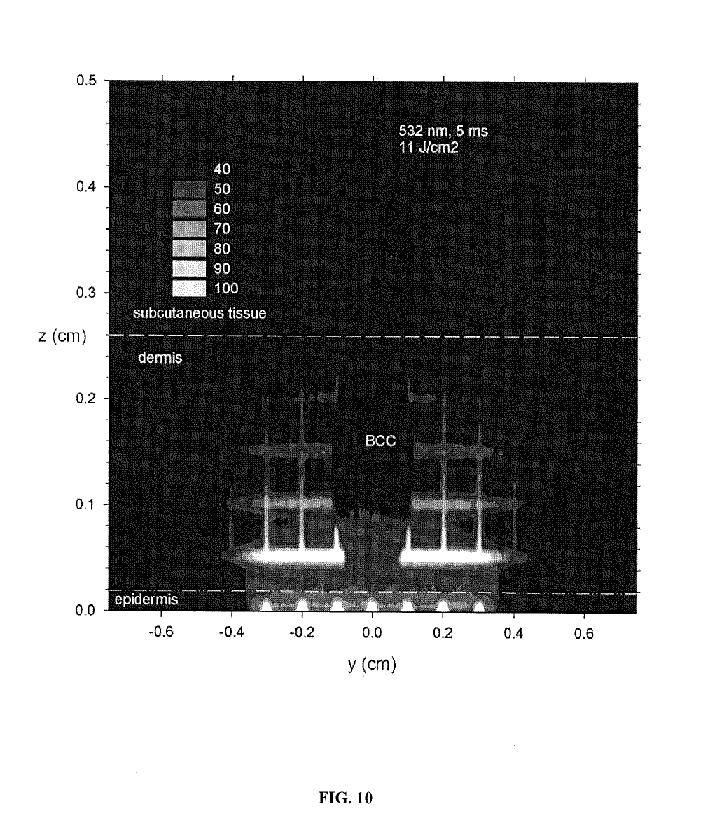

As another example of the invention, model calculations are performed for the case of a 1064 nm Nd:YAG laser. At this wavelength, as may be seen from Table I, the absorption of blood is lower, such that higher fluences are required to produce vascular injury, than was the case for the 585 nm laser. The absorption of blood is higher than the absorption of dermis at 1064 nm, so it is still possible to have preferential vascular injury. Scattering is significantly less at 1064 nm, such that the near-infrared radiation penetrates much more deeply. FIG. 9A depicts the results of calculations for a 7 mm diameter collimated beam with fluence of 80 J/cm² and duration 2.0 ms. Heating of the ascending and descending vessels to the 70° C. extends to 2 mm below the tissue surface. However, there is coagulation of non-vascular tissue the upper dermis, possibly limiting drug diffusion into that layer.

In this and all of the previously-described model calculations, a collimated laser beam was assumed. In FIG. 9B, the effect of a focused beam is calculated. Focusing may be achieved, for example, by using a lens as ablation element. In this model calculation, the focal length (in air) of 5 mm. Because of tissue scattering, the effect on the calculated temperature profile is relatively small. This finding suggests that wavelength is a more important determinant of tissue effects than focusing.

In FIG. 9C, a spherical tumor that extends from the epidermal-dermal junction to the bottom of the dermis is included in the model calculation. Also, additional horizontal vessels are added to the model, to represent the presence of microvessels growing from the tumor nodule. Because the tumor itself has relatively low absorption coefficient, it is heated less than the surrounding dermis, although its microvascular support is heated. In this model calculation, the fluence of the 1064 nm Nd:YAG laser is 60 J/cm². Surface cooling using a sapphire ablation element maintained at 4° C. is used in the models of FIGS. 9A, 9B, and 9C.

Lastly, model calculations were performed for the case of a 532 nm KTP laser, a 5 ms duration pulse, and a sapphire ablation element at 4° C. Compared to the 585 nm PDL, heating is somewhat shallower. However, the extent and localization of damage may make this wavelength particularly useful for the treatment of superficial BCC.

The spot size of 7 mm is chosen for these model calculations because it is attainable using current laser technologies, is large enough that scattering does not limit penetration depth, and is small enough to be encompassed within the calculation volume. In practice, according to the present invention, there are no limitations on spot size.

Likewise, pulse durations other than 0.5 ms can be used effectively. As mentioned previously, microsecond time-domain pulses (pulse widths of hundreds of nanoseconds, for example) may damage microvessels, including the smallest capillaries, by mechanical damage resulting from the explosive vaporization of erythrocytes within the vessels. Therefore, it is possible to use microsecond pulses according to the present invention to damage subepithelial vessels and decrease subepithelial tissue permeability. Microsecond pulses may also be used with light-absorbing elements of a laser applicator to ablate epithelial tissue. However, as noted in the Background, millisecond-domain pulses have been used to cause shrinkage of BCC by targeting lesional vasculature. This process requires thermal damage to lesional vasculature, because mechanical damage (vessel rupture) may be followed by healing and recovery of the lesional vasculature. If the microvessels supporting the BCC tumor cells are only temporarily damaged, rather than permanently eradicated, the tumor may be less effectively treated. A highly advantageous aspect of the present invention is that the laser pulses that modify the permeability of the tissue environment of the tumor can also have a direct effect on tumor vasculature, thermally coagulating and causing irreversible injury to those vessels, and secondarily leading to tumor cell death. The effect of laser irradiation therefore may simultaneously injure the tumor, and modify the tissue to increase the tumor exposure to a therapeutic anti-cancer agent. These dual, simultaneous actions of the irradiation will have an additive effect, for a highly advantageous treatment. Therefore, pulse durations on the order of approximately 300 microseconds to 100 milliseconds are advantageous.

In addition to lasers, incoherent pulsed light sources such as filtered flashlamps or IPLs commonly used in dermatology may also be used. For example, an IPL with a filter providing output with selectivity for blood may be used. Selectivity for blood is achieved when the absorption coefficient for blood is greater than the absorption coefficient for dermis, at a wavelength or wavelengths of the light source. In the case of the IPL or pulsed diode lasers, the light source may be in the applicator itself, and closed loop circulation of water or other cooling fluid used to chill a waveguide of the applicator. An ablation element may be attached to a chilled waveguide, or the waveguide may itself be used as an ablation element. Also, while the lasers and light sources discussed herein are of a pulsed type, it is possible to use a continuous wave light source if it is scanned, shuttered, or otherwise configured to provide an exposure time on the tissue of approximately 300 microseconds to 100 milliseconds.

The model calculations presented herein have assumed an even, top-hat energy distribution or beam profile. In some embodiments, it may be advantageous to use a nonuniform beam profile. For example, if the applicator of FIG. 7 is used with a gaussian beam profile, such that the energy density is greater at the center of the beam, where the ablation element is located, a skin tumor may be treated at a higher fluence than the surrounding normal skin, with a possible advantageous increase in efficacy. According to the invention, the design of the ablation elements and light-absorbing elements will take into account the beam profile. For example, if it is desirable to have a relatively uniform amount of epidermal ablation over the area of skin in contact with the ablation element, the number, density, or amount of chromophore material in the light-absorbing elements may be reduced in areas of the ablation element exposed to higher energy densities, relative to the remainder of the ablation element.

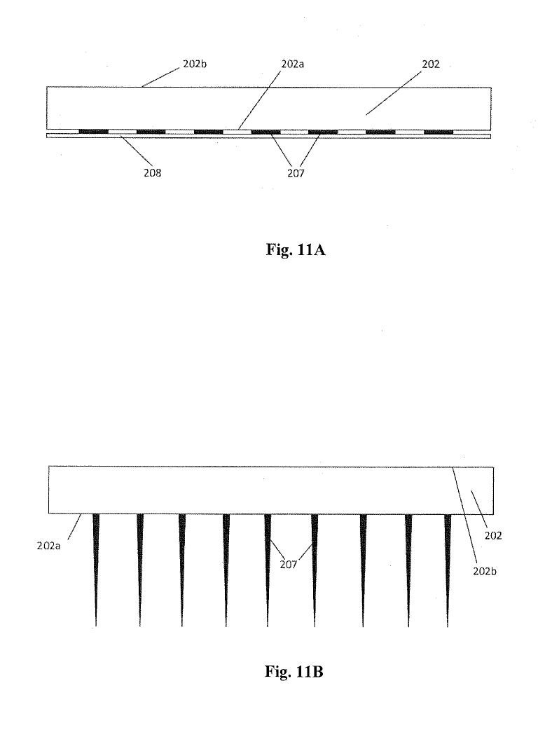

Other configurations of light-absorbing elements may be used, according to the invention. The shape of the elements may be changed, for example the elements may be made to project somewhat further towards the skin, to increase the surface area of the elements. In FIG. 11A, the light-absorbing elements (207) are disposed between the ablation element front surface 202a and a thin window 208. This configuration has the advantage of sealing the light-absorbing elements from tissue contact. If, for example, the thin window 208 is made of sapphire, it may be very thin yet strong and have excellent heat transfer capabilities for producing ablations. Optical quality sapphire wafers as thin as 100 μm are available, and may be used according to the invention. An applicator with a sapphire tissue-contacting surface may be readily cleaned and reused.

Also, with the light-absorbing elements completely sealed away from skin contact, a wider range of material may be used for said elements. For example, the light-absorbing elements may be made of a material that is less durable or less biocompatible. The light-absorbing elements may have a circular cross-sectional shape, or any other convenient shape.

FIG. 11B shows yet another configuration for light-absorbing elements. In this case, the elements are needle shaped to penetrate the tissue surface. Recalling the model results for the 1064 nm Nd:YAG laser, where heating of the upper layers of the dermis was found, the configuration of FIG. 11B would allow ablations to be performed into those upper layers, thus circumventing regions of diffuse, nonselective coagulation, for increasing drug diffusion into the dermis. The thin layer of coagulation around the ablation zones of the needles would provide for an advantageously bloodless treatment.

In an embodiment of the present invention, the light-absorbing element is carbon or a carbon material. Suitable carbons or carbon materials have properties that include high absorption coefficients for light and biocompatibility. Numerous carbons and carbon materials have been synthesized for engineering applications, and have a wide range of physical properties. The materials that are advantageous for the present application are identified by first considering the properties of natural crystalline carbon allotropes. Graphite, in which carbon atoms are sp² hybridized, has a very high and nearly constant absorption coefficient of approximately 2×10⁵ cm⁻¹ over the wavelength range of 600 nm to at least 2 microns, in contrast to the diamond allotrope which consists of sp³ hybridized carbons and which is transparent in the visible and NIR spectral range. The use of pyrolytic carbon as a surface material for prosthetic implants is well known in the art. Pyrolytic carbon has a very high absorption coefficient of a very wide range of wavelengths, making it a suitable chromophore for use with lasers operating throughout the visible and near infrared wavelength range so that the operating wavelength can be selected on the basis of optimal tissue penetration depth and extent of absorption by endogenous chromophores such as blood.

Another very advantageous chromophore is magnetite, or Fe₃O₄. Magnetite also has a very high absorption coefficient throughout the visible and near-infrared range, and is biocompatible. Magnetite powder can be formulated into paint for application to a surface of an ablation element. Other form of ablation elements can be made by adding magnetite to a ceramic material that is then shaped to make blunt projections, needles, or other shapes that are fixed to an ablation element.

As will be recognized, many different pigments and dyes may be used in the light-absorbing elements of the invention, depending on the light source. Examples include indocyanine green, methylene blue, rose bengal, and many other light-absorbing materials known in the art.

The ablation element may comprise quartz, glass, sapphire, optical quality plastic, or any other material that is substantially transparent to the laser radiation. The use of light-absorbing elements as an integral part of the ablation element of a laser applicator eliminates the potential safety or toxicity problems associated with applying dye or chromophore onto the tissue. Furthermore, use of the ablation element is more precise than application of a chromophore to the tissue, particularly when the tissue surface in the vicinity of a lesion may be smooth, rough, or damaged, thereby absorbing different amounts of an applied material.

An important aspect of be present invention is that it may be used with any topical or surface-applied drug or therapeutic agent, for an improved treatment of skin cancers and other lesions of the skin. Creation of ablation zones in the stratum corneum and all or part of the epidermis will increase permeability of the epidermis to any drug or agent, regardless of the drug's chemical properties, for example its lipophilic or hydrophilic nature, molecular size, molecular charge, or its formulation (solution, carrier, emulsion, cream, and the like). Likewise, damaging dermal vasculature and reducing uptake of drug from the dermis to systemic circulation will generally increase exposure time of the dermis to any drug. Drugs may include chemotherapeutic agents, cytotoxic drugs, antiproliferative agents, retinoids, vitamins, antioxidants, anti-angiogenic agents, immunomodulatory agents, photodynamic drugs, pro-apoptotic drugs, antimetabolite, COX inhibitors or any other agents that may be useful directly or indirectly in killing or damaging, or reducing the growth or proliferation of, tumor cells, malignant cells, dysplastic cells, diseased cells or abnormal cells.

A specific example includes vitamin D and its analogs, which recent research has shown to have immunomodulatory, antiproliferative, and prodifferentiative effects. This group of drugs includes calcipotriol (calcipotriene), a synthetic vitamin D₃ analog used for the treatment of psoriasis, and available in a 0.005% ointment or cream formulation (Dovonex, Warner Chilcott, Rockaway N.J.; Psorcutan, Intendis, Germany). Repeated application of topical calcipotriol over a period of several weeks has recently been reported in the medical literature to have some efficacy in treatment of actinic keratoses (AK), a premalignant or early form of squamous cell cancer (SCC) of the skin, in treatment of warts, benign viral tumors of the skin, and in treatment of Kaposi's sarcoma and cutaneous T-cell lymphoma. The naturally occurring active form of vitamin D₃, calcitriol (Vectical, Galderma, 3 mcg/g topical) has recently been approved in the US for treatment of psoriasis. Both calcipotriol and calcitriol have poor penetration through intact stratum corneum. When used according to the present invention, and applied to the site of a skin lesion after the skin tissue has been modified by epidermal ablation and dermal vascular injury, proliferative cells such as BCC cells may have much greater exposure to vitamin D analogs including calcitriol and calcipotriol, for a highly effective treatment.

Topical application of the retinoid tazarotene (0.1%) on a daily basis for up to 8 months has been reported to provide complete or partial results in treatment of BCC. Tazarotene (Tazorac, 0.05% or 0.1% gel, Allergan, Irvine Calif.) is approved as a topical treatment for psoriasis and acne; however retinoid drugs are also known to control the development and spread of cancer cells and cell proliferation. Tazarotene has limited skin penetration, due to the stratum corneum barrier, which may account for the lengthy treatment regime and incomplete efficacy for BCC treatment. All-trans-retinoic acid has shown antiangiogenic and anticancer properties when given intravenously. With the present invention, it is possible to apply all-trans-retinoic acid topically as a treatment for skin cancer.

Another example of a cytotoxic drug that may be used according to the present invention is a 6% solution of miltefosine (Miltex, Asta Medica, Germany). Miltefosine acts on cell membrane phospholipids and has been used with some reported efficacy in treatment of skin metastases in breast cancer and cutaneous T cell lymphoma, with daily application for at least several weeks. Miltefosine efficacy those skin tumors as well as BCC will increase with the tissue modification of the present invention.

A drug that may be particularly advantageous when used according to the present invention is mitomycin C (MMC). MMC (Mutamycin, Bristol-Myers Squibb, Princeton N.J.) is a chemotherapeutic quinone alkylating agent that inhibits DNA synthesis to prevent proliferation of malignant cells. It has been in regular use for scar prevention in ENT surgery and ophthalmology. MMC is hydrophilic and is applied topically in an aqueous solution to surgical wounds. For skin surgery, cotton pledgets soaked with 0.4 mg/5 cc MCC have been applied for 4 min after excision of keloids to prevent their regrowth. As a hydrophilic drug, it does not permeate intact epidermis. The present invention makes it possible for MMC to be used for treatment of BCC and other skin lesions by removing the stratum corneum and all or part of the epidermis, so that MMC may readily diffuse into the dermis. Also, importantly, an aspect of the invention is the bioreductive activation of MMC, which will enhance the toxicity of the drug and the efficacy of the treatment. The bioreductive activation of MMC is a result of the hypoxic environment resulting from vascular injury, as described in detail in the section below on bladder cancer treatment according to the present invention. Thus, MMC has advantages of proven efficacy as an antineoplastic drug, its history of safe use on epithelial (skin and mucosal) tissue, and it bioreductive activity.

Other quinones, N-oxides, nitroaromatics, uracils, and cobalt(III) complexes, for example porfiromycin, CI-1010, tirapazamine, 90CE, TX402, NLCQ-1, OFU001, and CTC-96, have been studied as bioreductive drugs and may be useful in accordance with the present invention, for treatment of skin cancer.

Yet another group of therapeutic agents that may be used advantageously according to the present invention are COX inhibitors. Examples include diclofen, a nonsteroidal anti-inflammatory drug and nonspecific COX inhibitor that is used in a 3% gel formulation (Solaraze, PharmaDerm, Melville N.Y.) for treatment of AK; celecoxib, valdecoxib, and sulindac, among others.

Antioxidants have been shown to have promise in treatment and prevention of cancer. Topical treatment with resveratrol, an antioxidant found in grapes and berries, black raspberry extract, pomegranate seed oil, grape seed proanthrocyanidins, beta carotene, ascorbic acid, and lycopene are examples.

The above is only a partial listing of drugs or therapeutic agents that are useful according to the present invention. Also, of those described, alternative formulations or dosages may prove advantageous in treatment of tissue modified by the laser treatment of the invention. Furthermore, combinations of drugs may be used with said laser treatment.

An important aspect of the present invention is its versatility, with respect to light source and therapeutic agent. Some examples of the present invention are as follows:
A small 3 mm surface diameter nodular BCC is diagnosed. The patient's tumor is treated with a 585 nm PDL with 0.5 ms pulse duration, using an applicator configured as in FIG. 7, with ablation element 10 mm in diameter, a uniform beam profile, and irradiated spot size 18 mm diameter. The sapphire ablation element has light-absorbing elements that are 400 μm in diameter and spaced 1 mm apart in a cubic arrangement. Laser fluence of 9 J/cm² is used, with a single laser pulse and cold air cooling. Immediately after irradiation, the tumor is treated with a pledget soaked in mitomycin C at 0.4 mg/ml for 10 min. The tumor site is thoroughly irrigated with saline and bandaged until healed. The patient returns for followup examination 4 weeks later, and is retreated using the same parameters if residual tumor is found.
A large superficial BCC is diagnosed. The patient's tumor is treated with a 532 nm KTP with 1 ms pulse duration, using an applicator configured as in FIG. 4A, spot size of 10 mm diameter. The tumor and peri-lesional zones are treated with a fluence that produces thermal injury to the epidermis and purpura over the entire irradiated spot. A zone of normal-appearing tissue around the peri-lesional zone is treated with a fluence that produces purpura over the entire irradiated spot, but little or no epidermal injury between ablation zones. Within 5 min after irradiation, the tumor is treated by application of a thin layer of tazarotene over the irradiated areas, and bandaged. The patient is instructed to apply a layer of tazarotene daily over the irradiated areas for the next week, or until the treatment site heals. The patient returns for followup examination 3 weeks later, and is retreated if residual tumor is found.
A 5 mm surface diameter nodular BCC is diagnosed. The patient's tumor is treated with a 1064 nm Nd:YAG with 2 ms pulse duration, using an applicator configured as in FIG. 4A, with irradiated spot size 5 mm. Spots are overlapped as necessary to treat the lesion and a 5 mm peri-lesional zone. The sapphire ablation element has light-absorbing elements in the form of 2 mm long carbon-coated needles that are 200 μm in diameter and spaced 0.75 mm apart in a cubic arrangement. Laser fluence of 80 J/cm² is used, with the ablation element maintained at 4° C. when treating the surrounding normal skin. Immediately after irradiation, diclofen gel is applied to the tumor surface. The patient is instructed to apply a layer of diclofen gel daily over the tumor surface until the treatment site heals. The patient returns for followup examination 3 weeks later, and is retreated if residual tumor is found.
   It is recognized that the present invention may be used to treat not only BCC, but many other benign and malignant skin tumors and premalignant lesions, including but not limited to seborrheic keratosis, actinic keratosis, Bowen's disease, keratoacanthoma, squamous cell carcinoma, and cutaneous lymphoma.

Furthermore, the device of the invention may be used in applications unrelated to cancer. For example, an applicator configured as in FIG. 4A may be used with a KTP laser at low (subpurpuric) fluence to create a mild thermal injury to the dermis along with ablation zones in the epidermis, followed by application of an antioxidant such as black raspberry extract, for a highly effective skin rejuvenation treatment. Another example is the use of an applicator of the invention with a laser wavelength not selective for blood, for example 1450 nm, along with a vitamin B5 (pantothenic acid) as a treatment for wrinkles. Yet another example of an aesthetic application of the invention is the use of the applicator with an ablation element having light-absorbing elements of diameter approximately 500 μm in diameter, separated by 750 μm in a hexagonal pattern, used with a PDL at subpurpuric fluence as a treatment for enlarged pores, with or without a topical agent. As may be appreciated, very many advantageous applications of the devices and methods of the invention are possible.

### Bladder Cancer

Methods and devices are described for the treatment of bladder cancer, involving use of electromagnetic radiation to alter the permeability of at least one layer of the bladder wall and administration of a chemotherapeutic or anticancer agent to the bladder lumen. According to one method of the invention, a laser or other radiation source is used to induce damage to suburothelial blood vessels of the bladder of a patient with bladder cancer, and a chemotherapeutic drug is then instilled into said bladder. In advantageous configurations, the suburothelial blood vessels are the blood vessels of the lamina propria of the bladder wall.

According to another method of the invention, a laser or other radiation source is used to simultaneously induce damage to the urothelium and suburothelial blood vessels of the bladder in a patient with bladder cancer, and a chemotherapeutic drug is then instilled into the bladder. Damage to the urothelium induced by the radiation may be of a continuous or discontinuous nature. In advantageous configurations, the urothelium damage is discontinuous.

In one embodiment of the invention, the device of the invention may be a visible or near-infrared laser or light source adapted to be connected to an optical fiber that can be inserted into the working channel of a cystoscope, such that the distal end of the fiber can be positioned at or near the urothelial surface of the bladder to deliver radiation to damage the urothelium and/or suburothelial blood vessels prior to or during instillation of a chemotherapeutic drug.

According to one aspect of the invention, treatment increases the exposure of tumor cells located in the urothelium and suburothelium to a chemotherapeutic drug. According to another aspect of the invention, treatment causes regression or destruction of malignant or premalignant tissue located in the urothelium and suburothelium. According to yet another aspect of the invention, treatment prevents growth of tumors from tumor cells adhering to the bladder wall. According to still another aspect of the invention, treatment increases the cytotoxicity of certain chemotherapeutic agents. An embodiment of the present invention provides a method and device for reducing the recurrence and progression of cancer of the bladder. Another embodiment of the present invention is to provide a method and device for treating cancer of the bladder that may be used under local anesthesia.

The methods and devices of the present invention can be adapted for the treatment of cancers other than bladder cancer. For example, a method and device of the invention may be used for a highly advantageous treatment of skin cancer, precancerous lesions, proliferative skin lesions and other dermatologic conditions.

The present invention is described in detail using model calculations to demonstrate the interaction of light with tissue, and the interaction of tissue with drug. Model calculations for the interaction of light with tissue are used to show the extent and degree of heating and thermal injury in the tissue, and model calculations for the interaction of drug with tissue are used to show the effect of tissue injury on drug pharmacokinetics, for examples of the invention.

Model calculations are disclosed for the bladder as the target organ of the treatment of the invention. First, Monte Carlo calculations of photon transport in biological tissue followed by heat transport analyses are disclosed that lead to an understanding of how urothelial and suburothelial tissue can be heated or damaged by electromagnetic radiation. This heating of or damage to urothelium and/or suburothelium can be used to alter, increase and/or decrease the permeability of the bladder wall in a useful manner, as will be shown subsequently in pharmacokinetic model calculations.

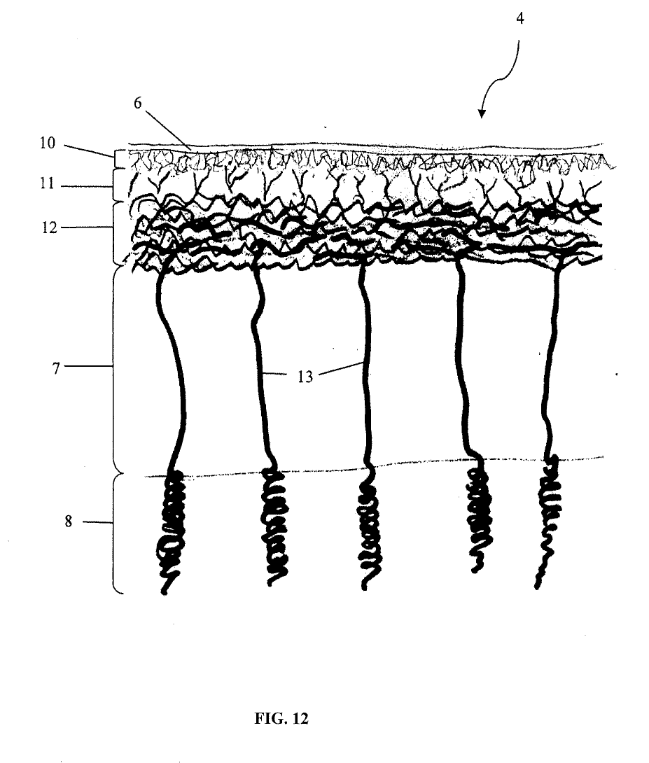

For an understanding of the present invention, the urothelium and the blood vessels of the bladder wall are most relevant, as they relate to drug distribution in tissue. FIG. 12 is a schematic drawing of the vasculature of the bladder. Perpendicular vessels 13 from the advential/serosal plexus travel through the muscularis propria 8 and the lamina propria 7 to supply the mucosal plexus 12. The vessels of the mucosal plexus 12 are oriented in a predominantly horizontal direction, and comprise capillaries, as well as arteries and veins. The mucosal plexus is connected in turn to the superficial capillary plexus 10 by perpendicular arterioles and venules 11. The capillaries of the superficial plexus are located directly underneath the urothelium 6 and are densely packed. The tissue comprising the superficial capillary plexus and underlying perpendicular connecting vessels has a thickness of about 300 μm, and the mucosal plexus is about 850 μm thick. The urothelium is about 160 μM thick and contains no blood vessels. The total thickness of the lamina propria is variable, but ranges up to about 3 mm.

Drugs can travel through tissue either by diffusion, by being taken up by blood vessels, or both. According to embodiments of the present invention, the uptake of drug by blood vessels in the bladder wall can be inhibited by an injury to the vessels of the suburothelial tissue, specifically the lamina propria. The vascular plexuses in the upper part of the lamina propria (the superficial capillary plexus and the mucosal plexus) comprise the first blood vessels that a drug molecule diffusing from the urothelium encounters, and are densely populated. Below the mucosal plexus, the lamina propria has relatively few capillaries and drug molecules must travel mainly by diffusion to reach the capillary network of the muscle layers and advential/serosal plexus. Thus, if the vascular plexuses of the upper lamina propria are disabled, drug elimination from the urothelium will proceed mainly by the relatively slow process of diffusion through both the urothelium and the lamina propria, layers corresponding to the first few millimeters of bladder wall tissue and the location of superficial bladder tumors. According to embodiments of the present invention, it is advantageous to avoid injury to the muscle layers, as they are involved in the bladder voiding function. Also, it is unnecessary to injure the vessels of the muscle layers, because muscle invasive bladder cancer is generally treated with systemic chemotherapy and/or ionizing radiation, rather than intravesical chemotherapy.

According to embodiments of the present invention, uptake of drug by blood vessels can be inhibited by photothermal injury to those vessels. The vessels of the superficial capillary plexus are sufficiently small that it would be difficult to confine heat in those structures, unless irradiated with pulses of short duration (on the order of tens of microseconds) that may have a tendency to rupture larger vessels in the lamina propria. However, the superficial capillary plexus is supplied by arterioles and venules that may be effectively heated by millisecond-regime pulses. In addition, the smaller arteries and veins comprising the mucosal plexus are of a readily targetable size with millisecond-regime pulses. Inducing photothermal damage to the mucosal plexus and/or the perpendicular arterioles and venules can be expected to be sufficient to affect the overall perfusion of the superficial capillary plexus, whether or not there is direct damage to the superficial capillaries, since those capillaries are supplied by the underlying vessels. The relatively large perpendicular vessels of the deeper lamina propria are also potential targets, as they supply the mucosal plexus. According to the present invention, therefore, all of the blood vessels of the lamina propria are targets for photothermal injury. In an advantageous implementation of the invention, the blood vessels within the mucosa and lamina propria with diameter greater than about 20 μm are targets. Also, in an advantageous implementation of the invention, the vessels of the muscularis propria, adventia, and serosa should remain substantially uninjured by the treatment.

Targeting the mucosal plexus and perpendicular arterioles and venules connecting it to the superficial capillary plexus requires absorption of sufficient radiation by those structures at their depth from the mucosal surface to produce thermal injury. For efficient and selective heating of the targets, heat should be confined in the target during the laser pulse. The results of interaction of light with the tissue is found herein using Monte Carlo photon transport calculations. The results of the Monte Carlo calculation are then used in a heat transfer analysis to find the temperature within the tissue after irradiation.

Herein, optical properties of bloodless, unpigmented skin are used as an estimate for those of the avascular urothelium. For the remaining layers (lamina propria and muscularis propria) optical constants for in vitro bladder tissue are modified to account for the higher blood content of living tissue. Specifically, a 1% blood contribution is added to absorption coefficients of in vitro bladder specimens to account for loss of blood from incised vessels. Table II below lists the constants used in the model calculations performed and described herein. It is recognized herein that muscularis propria optical properties may actually differ from those of lamina propria, however in treatment of bladder wall according to the present invention the effect of electromagnetic radiation is most important for the urothelium and lamina propria layers, where the tumors to be treated may be located, and differential optical properties of muscularis propria can be neglected without significantly affecting the results.

TABLE II
Optical constants of bladder tissue.

| | | | | lamina propria | | | |
|---|---|---|---|---|---|---|---|
| | blood vessels | | | (muscularis propria) | | | |
| | μₐ | μₛ | | μₐ | μₛ | | urothelium |
| λ (nm) | (cm⁻¹) | (cm⁻¹) | g | (cm⁻¹) | (cm⁻¹) | g | μₐ (cm⁻¹) |
| 532 | 225 | 692 | 0.96 | 7.06 | 145 | 0.90 | 0.527 |
| 585 | 177 | 764 | 0.95 | 4.28 | 146 | 0.92 | 0.371 |
| 755 | 2.3 | 843 | 0.98 | 1.28 | 128 | 0.92 | 0.254 |
| 800 | 3.3 | 815 | 0.98 | 1.35 | 124 | 0.92 | 0.249 |
| 1064 | 4.3 | 654 | 0.97 | 0.59 | 105 | 0.92 | 0.244 |

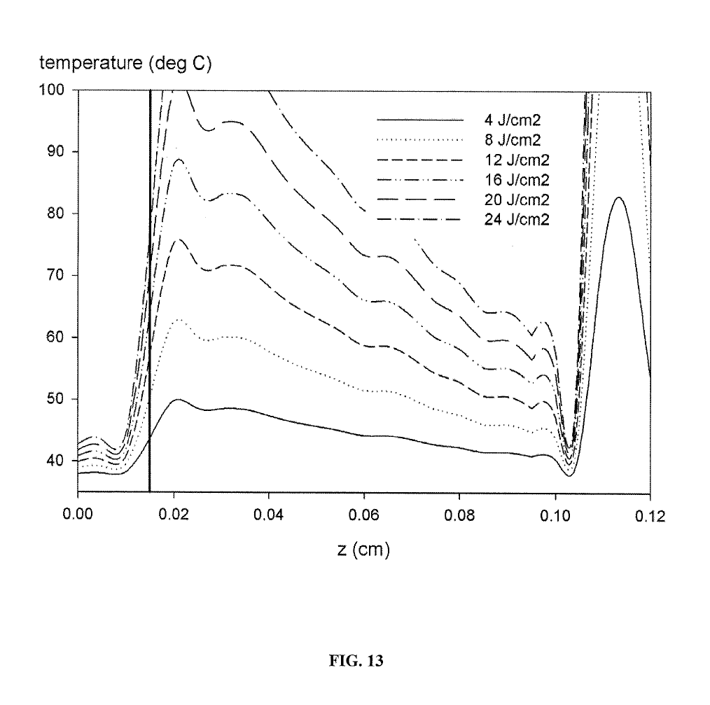

FIG. 13 shows the results of model Monte Carlo and heat transfer calculations for the interaction of radiation from a 585 nm pulsed dye laser with pulse width 0.5 ms and a collimated, 3 mm diameter irradiated spot on bladder tissue. This laser was selected for the calculation because it is commercially available (Candela Corporation, Cynosure, Inc.) and is known to be capable of producing selective injury to blood vessels in skin. The calculation is over a 2.25 cm³ rectangular volume (area 1.5 cm×1.5 cm, depth 1.0 cm) and has a resolution of 50 μm in each direction. For clarity the figure depicts only the portion of the calculation volume where tissue heating is localized. The energy distribution of the 3 mm spot is Gaussian, corresponding to radiation from a bare laser fiber held approximately 6 mm from the urothelial surface of the bladder. The bladder in the model is filled with room temperature saline. The tissue model includes a urothelial layer that is 150 μm thick, and a cylindrical blood vessel with diameter 100 μm, centered 1.15 mm under the urothelial surface. This vessel is of a size, depth and orientation consistent with vessels near the bottom of the mucosal plexus of the bladder, i.e., the deepest targets At the lowest fluence tested in the calculations, 4 J/cm², the intravascular temperature is marginally sufficient to induce at least temporary coagulation of the vessel contents. At higher fluences, complete coagulation is found, and at the highest fluences tested, the vessel may be heated to the point of rupture and hemorrhage, even with the millisecond-domain pulses modelled herein. The heat transfer calculations does not take into account phase changes, therefore 100° C. is the maximum temperature represented. The temperature corresponds to the time at the end of the laser pulse. For this and other model calculations described herein, one million individual photons were launched in the photon transport calculation.

Damage to cells in the vicinity of the basement membrane can be inferred from the calculations of FIG. 13 by the increasing temperature at a depth of 150 μm (approximate junction of urothelium and lamina propria, or approximate location of the basement membrane) with increasing fluence. At a fluence of 4 J/cm², this temperature is less than 45° C. As the fluence increases to 20 J/cm², the junctional temperature reaches 70° C., and it is approximately 78° C. at a fluence of 24 J/cm². The critical temperature for irreversible thermal injury of tissue shows some variation with tissue type, and the specific thermal damage parameters characterizing bladder tissue are not known, however cellular soft tissue is typically damaged when exposed to temperatures of between 70 and 80° C. for times on the order of milliseconds. The construction herein of this model for the interaction of bladder wall with light allows the degree and spatial extent of photothermal injury to specific components or layers of the bladder wall to be correlated with the output parameters of different lasers and light sources.

The model developed herein is further enhanced by adding additional blood vessels representative of the vascular architecture of the tissue layers, as is represented schematically in FIG. 12. Specifically, multiple 50 μm diameter blood vessels oriented with long axes perpendicular to the urothelial surface are located with upper and lower ends at 300 and 450 μm below the surface, respectively. These 50 μm segments represent the ascending and descending arterioles and venules connecting the superficial vascular plexus to the mucosal plexus, and are spaced 500 μm apart. Parallel to these short segments are multiple longer segments starting at 450 μm and descending to deeper layers of the bladder wall, with diameter 100 μm and separated by 1 mm. These longer segments represent ascending and descending vessels of the lamina propria. Finally, vessels oriented parallel to the tissue surface and representative of the horizontal vessels of the mucosal plexus are centered 475, 850, and 1150 μm below the surface, with diameters of 50, 100, and 200 μm, respectively. This set of horizontal and perpendicular vessels represents the targets of photothermal injury in calculations using this fully vascularized mathematical model.

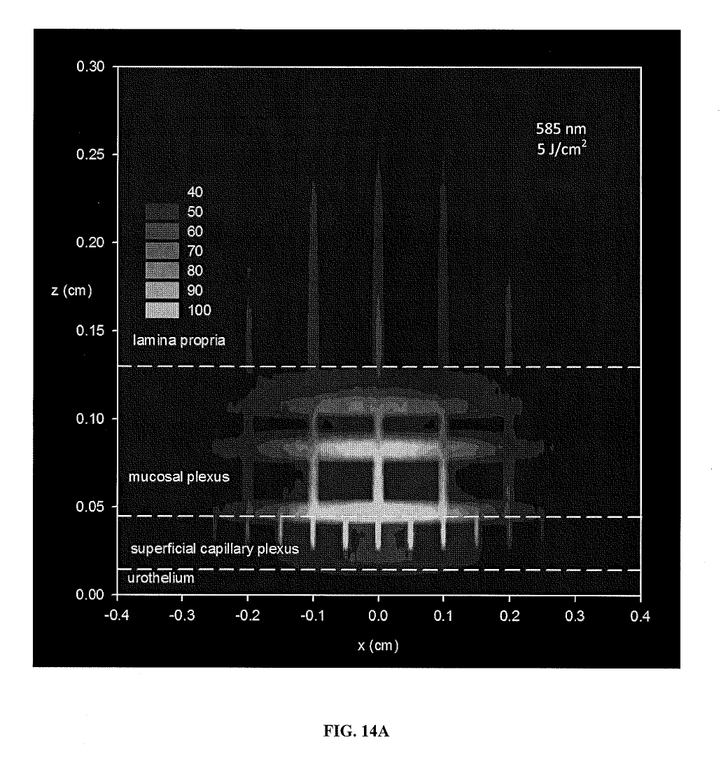

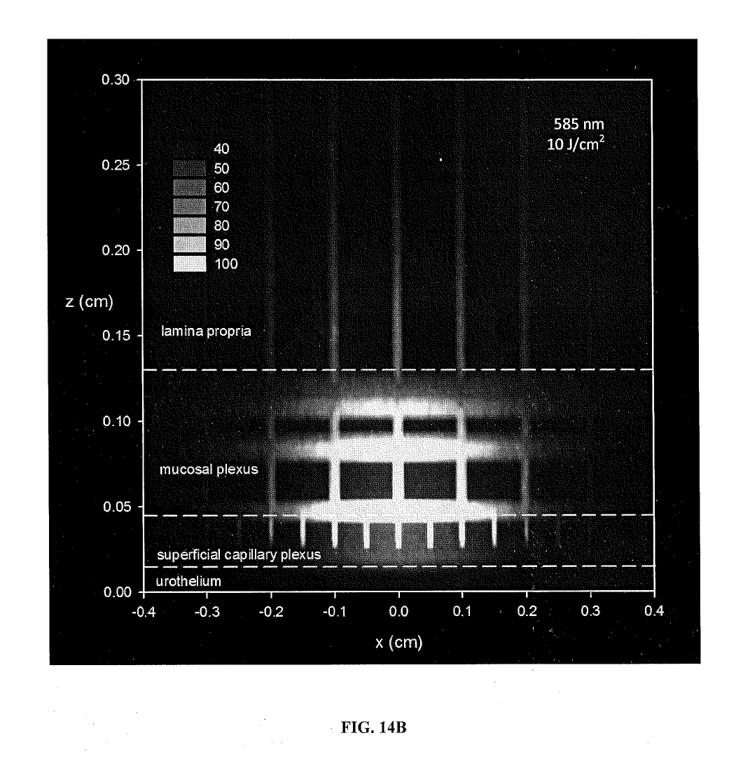

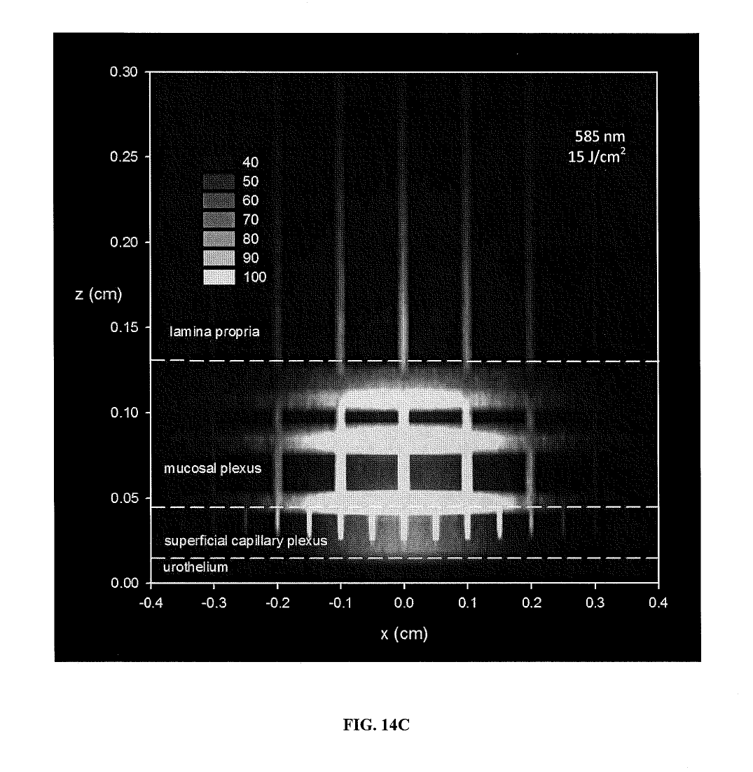

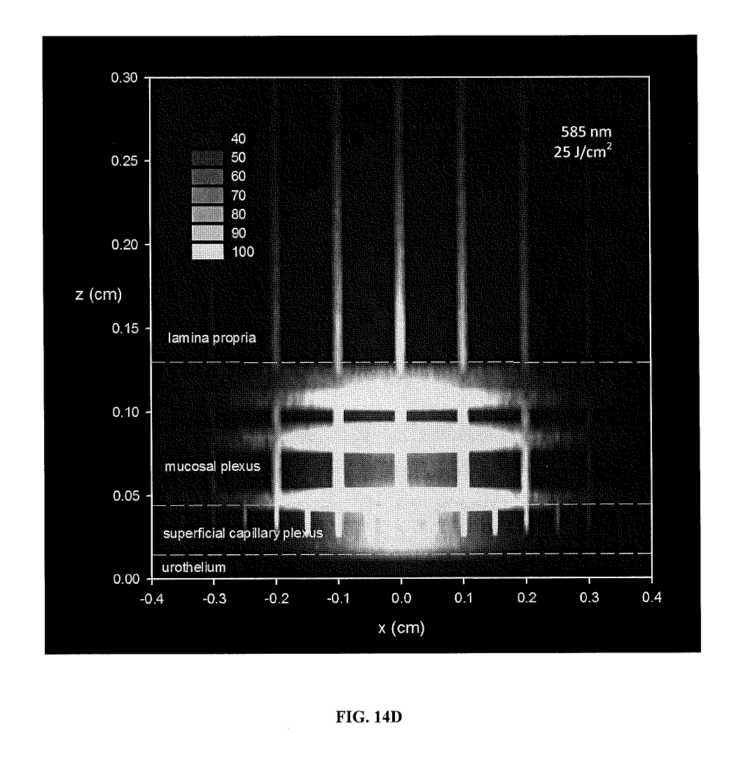

Results are shown in FIGS. 14A-4D for representative fluences of 5, 10, 15, and 25 J/cm², respectively, from a 585 nm pulsed dye laser with pulse duration 0.5 ms, and a 3 mm diameter Gaussian irradiated spot on the lumenal surface of a saline-filled bladder. The results depict a cross-section of the bladder wall down to 3 mm, corresponding to urothelium and lamina propria in a bladder region with relatively thick lamina propria. The representative vessels of the superficial capillary plexus and mucosal plexus of the upper lamina propria are seen to reach temperatures consistent with coagulation at all fluences, although the spatial extent of coagulation increases with fluence. For the highest fluence (FIG. 14D, 25 J/cm²) the calculation indicates that most of the perpendicular vessels of the lamina propria will be thermally injured, as shown in the calculation of FIG. 13. Thus, pulsed dye laser fluences that are consistent with thermal injury to the urothelium may at the same time produce significant vascular injury. At low fluence, for example at about 5 J/cm² (FIG. 14A), where urothelial layer injury may be insignificant, vascular injury is reduced in the deeper lamina propria but still significant in the mucosal and superficial capillary plexuses. These calculations represent the results of delivery of single pulses of radiation to a site on the bladder wall.

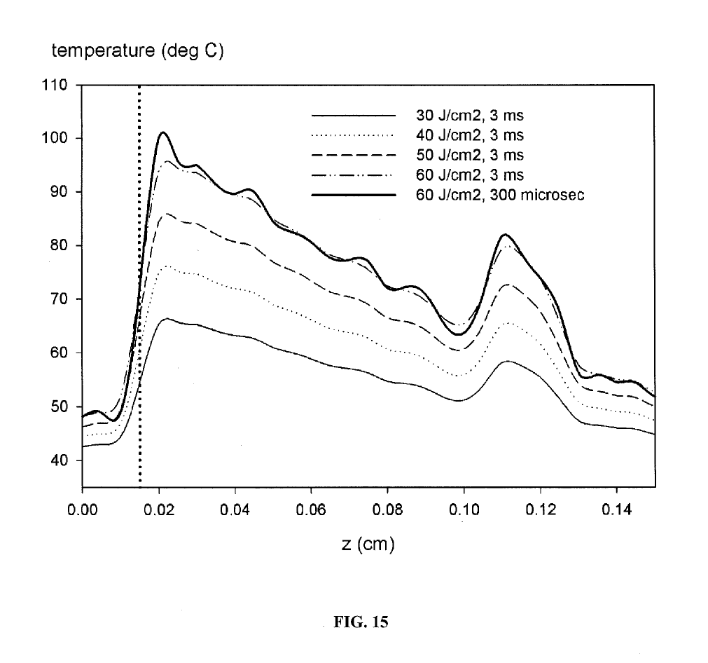

FIG. 15 shows results of model calculations for treatment of a bladder for the example of a flashlamp pumped alexandrite laser operating in the near-infrared at 755 nm. The absorption coefficient of blood is considerably reduced at that wavelength, although it is still higher than that of bladder tissue. Consequently, the calculations indicate that relatively high fluences (≧60 J/cm²) are required to produce temperatures≧70° C. near the junction between urothelium and lamina propria, with pulses of either 3 ms or 300 μs. Pulses in the millisecond or microsecond-domain are readily produced by flashlamp pumped alexandrite lasers developed for hair removal but also useful for certain vascular lesions (Candela Corporation, Light Age).

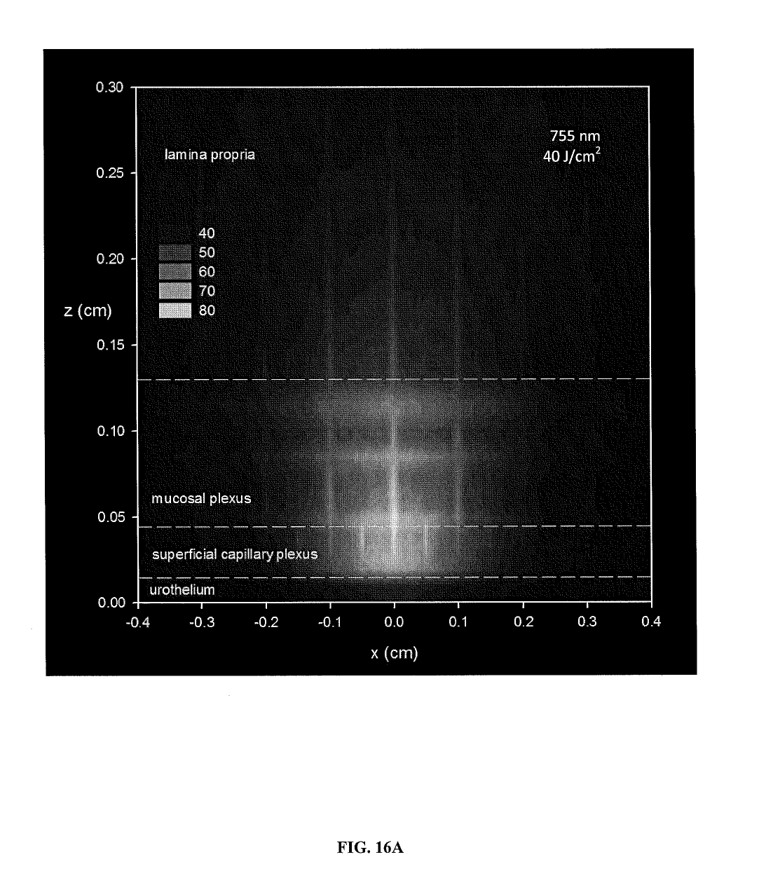

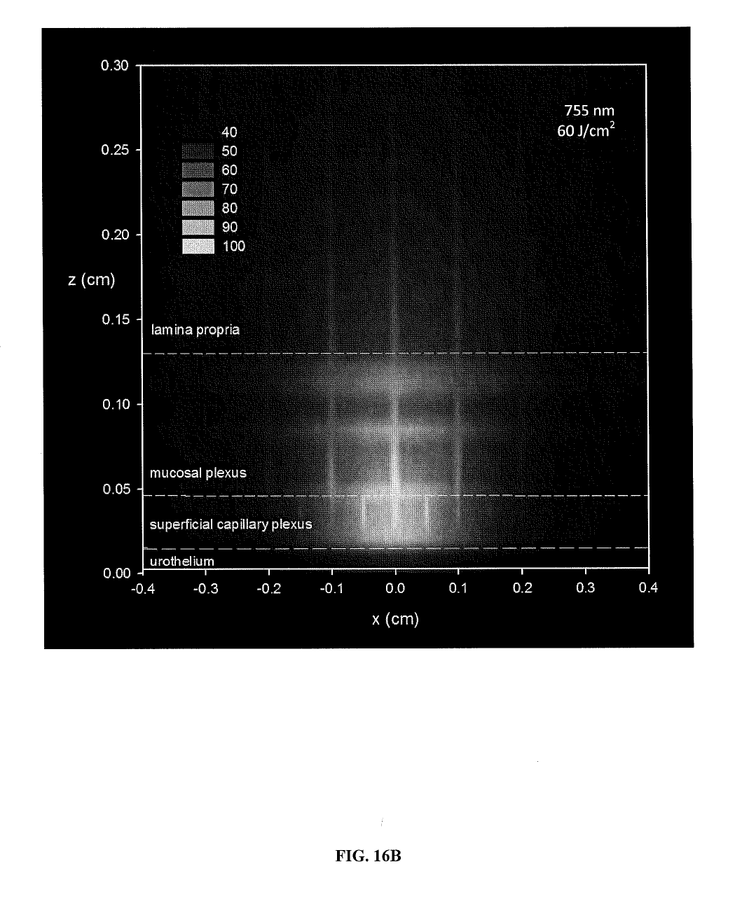

FIGS. 16A and 16B are contour plots that show results for an alexandrite laser with 3 ms pulse duration and fluence 40 and 60 J/cm², respectively. As a result of the deeper penetration of 755 nm light, fluences that correspond to coagulation of representative vessels in the superficial capillary plexus and mucosal plexus produce relatively more concomitant heating of the deeper lamina propria vessels, than does 585 nm light. This deeper heating may be advantageous in some situations, such as in the treatment of deeper residual tumor cells. This calculation (and others herein reported as contour plots) uses the same fully vascularized model with multiple vessels in the lamina propria described in detail above for the 585 nm pulsed dye laser, with only the wavelength, pulse duration and pulse energy of the laser and wavelength-dependent tissue optical properties changed as appropriate.

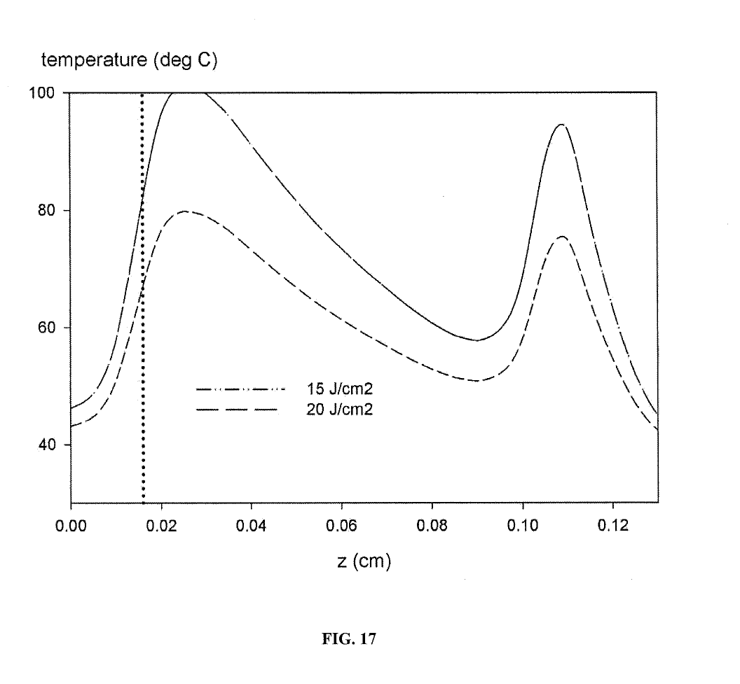

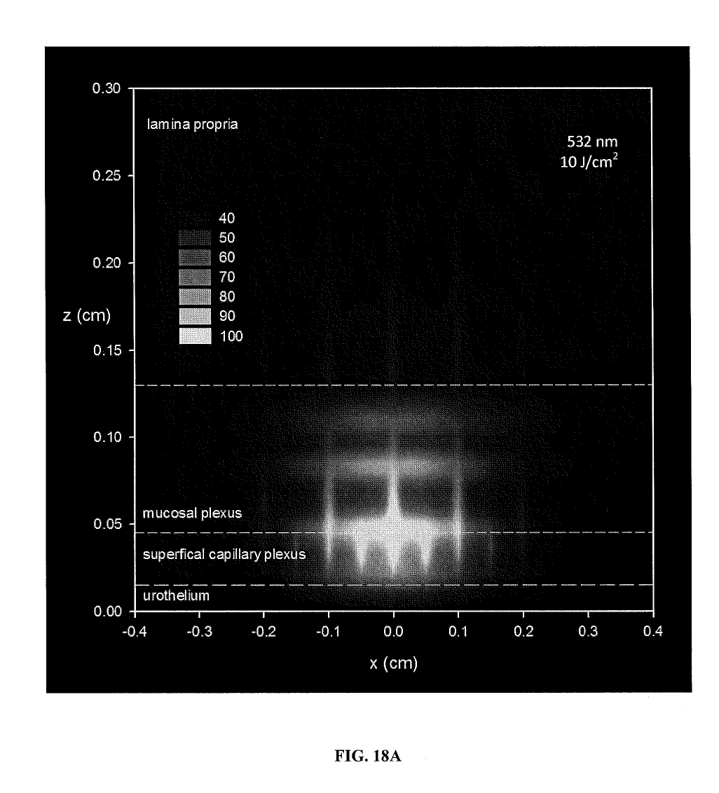

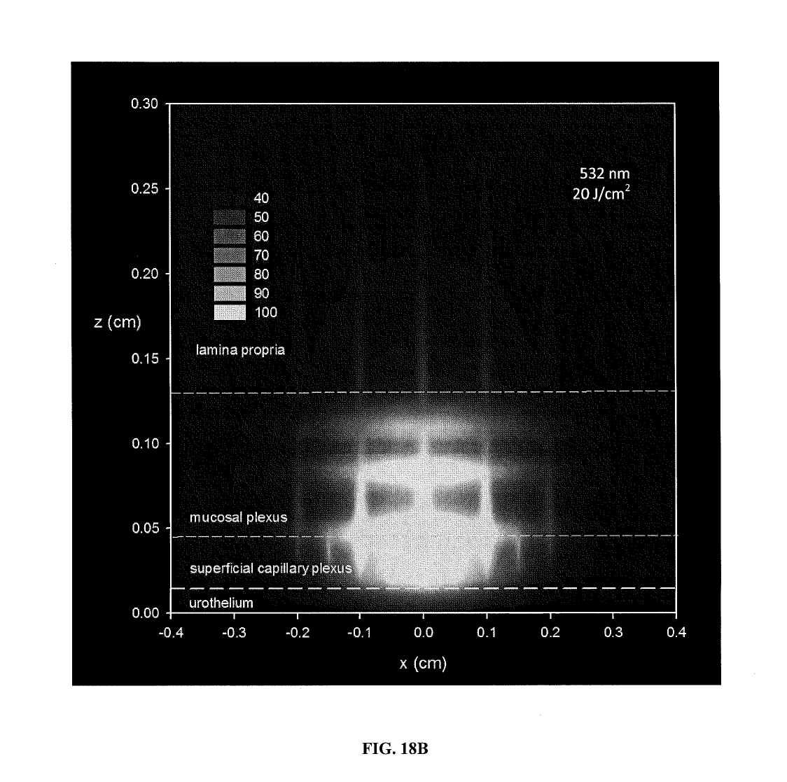

FIGS. 17, 18A, and 18B show results of the use of a KTP laser operating at 532 nm and 15 ms as modelled in the next example. The KTP laser is commercially available (Quantel Derma) and when configured for high pulse power and millisecond-domain pulse durations, is well known as a vascular targeting laser in dermatology. The graph of tissue temperature versus depth as a function of fluence (FIG. 17) indicates that the cells in the vicinity of the basement membrane may reach a temperature of over 70° C. at the end of a 20 J/cm² pulse. This result indicates that this or higher fluence may induce urothelial layer damage. According to the results of calculations using the fully vascularized model (FIGS. 18A and 18B), vascular injury localized to the mucosal and superficial capillary plexuses will be produced at fluences lower than that required to induce substantial urothelial injury. The relative extent of vascular injury in the deeper lamina propria is much less than was the case for the 755 nm alexandrite laser. The KTP laser, like the pulsed dye laser, may be advantageous when it is desirable to efficiently coagulate vessels of the more superficial lamina propria with reduced heating of deeper tissue. However, it may be necessary to use laser pulse durations in the millisecond domain (one millisecond to tens or hundreds of milliseconds) or microsecond domain (one microsecond to hundreds of microseconds) to selectively damage vessels of the size included in the model of these examples. Millisecond domain pulses are advantageous to minimize rupture of vessels.

Alternatively, a continuous wave or quasi-continuous wave high pulse repetition rate laser (for example, a quasi-continuous wave KTP laser) can be used if it is scanned across the tissue so that the tissue exposure time is in the millisecond or microsecond time domains. Such scanned vascular lasers are well known in dermatology. For applications in treatment of tissue of internal organs, the use of pulsed lasers may be simpler and hence more advantageous. Pulses of light with microsecond-domain or even shorter duration (for example Q-switched lasers with nanosecond-domain pulses, including the Q-switched KTP laser, or the coaxial flashlamp-pumped dye laser) can be used to produce photothermal injury selective to vasculature, although the injury may be more structural than thermal in nature, due to the explosive vaporization of cellular and subcellular structures that occur when heat is confined by very short pulses.

This structural damage, which may be seen as vessel rupture or hemorrhage, can occur at even relatively low pulse energies, unlike the situation with longer pulses, where hemorrhage generally will be observed with increasing pulse energy and is present with significant vascular coagulation. Structural damage to vessels induced by nanosecond or microsecond-domain pulses is generally avoided in treatment of cutaneous vascular lesions with lasers, as such damaged vessels are capable of healing and the treatment is hence less effective in producing long-term eradication of lesions, the goal of clinical treatment of portwine stain birthmarks, hemangiomas and the like. Herein, according to the present invention, it is recognized that a temporary elimination of the vessels or blood flow in the vessels is sufficient to affect the reduction in tissue permeability, however, so even the shortest time domains may be useful.

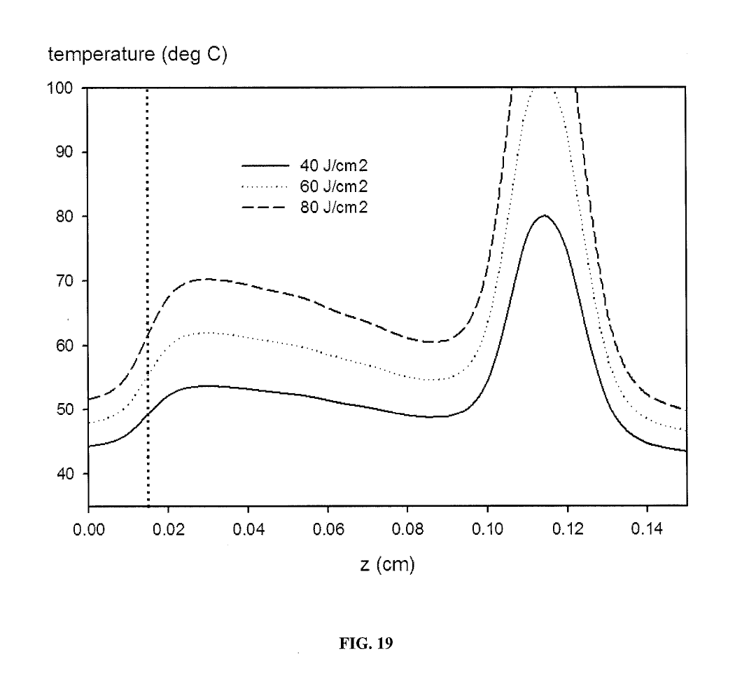

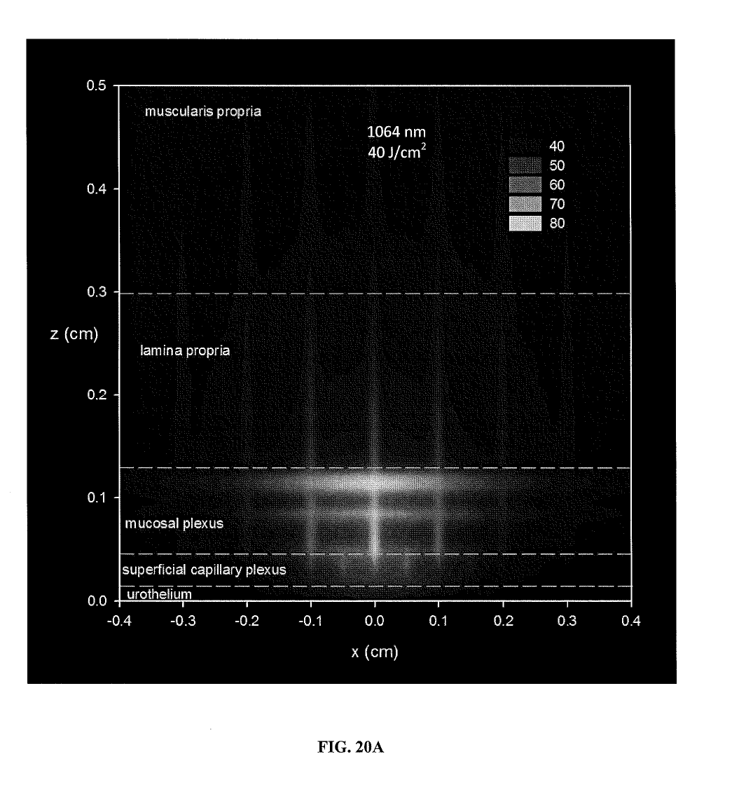

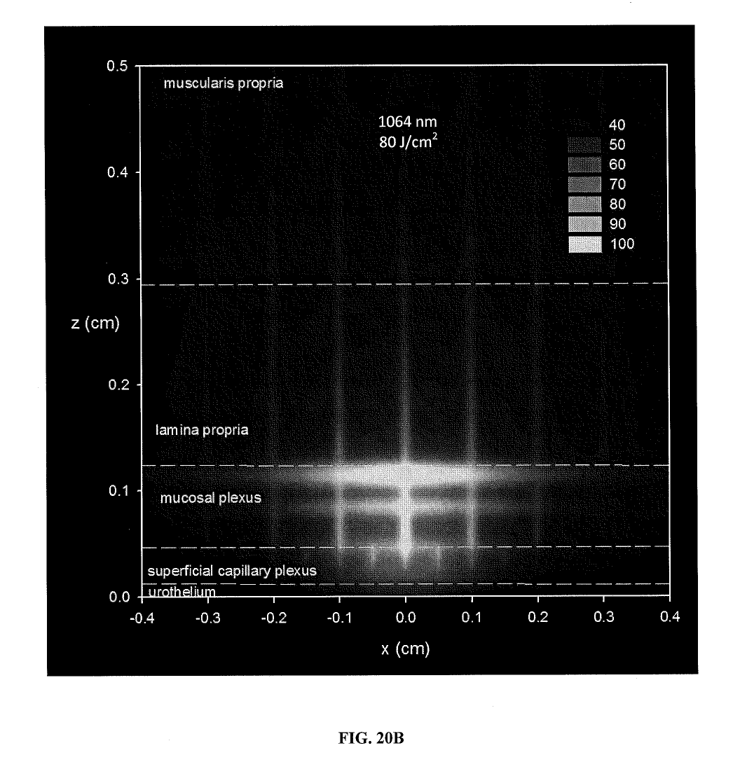

FIGS. 19, 20A, and 20B show results of a model using a flashlamp-pumped 1064 nm neodymium YAG (Nd:YAG) laser operated with a pulse duration of 15 ms. This pulsed laser (available from, e.g., Candela Corporation) is of a type that is used for hair removal, and is configured and constructed differently from a continuous wave Nd:YAG laser of the type commonly used in surgery where tissue is exposed to continuous irradiation for times on the order of seconds to coagulate volumes of tissue in the beam path. Here, the model results indicate selective vascular injury in the lamina propria of the bladder with the Nd:YAG pulsed laser. There is relatively little heating of the urothelium, however, even at high fluences (FIG. 19).

FIGS. 20A and 20B show results of calculations using the fully vascularized model, and depicts a cross section of the bladder wall that includes urothelium, lamina propria, and also muscularis propria. The model calculations indicate that pulsed light at 1064 nm can heat vessels through the full thickness of the lamina propria. Hence, it is determined that the pulsed Nd:YAG laser may be advantageous in producing deeper vascular injury in the bladder for treatment of more invasive tumor, but that it may be relatively ineffective in producing urothelial injury. The use of the Nd:YAG laser may also require greater care to prevent injury to the muscularis propria than is required for alexandrite, KTP or pulsed dye lasers.

An aspect of the invention is that the photothermal injury to suburothelial vessels that alters the permeability of the bladder wall for improved pharmacokinetics, also has a direct effect on tumors and premalignant lesions. The direct effect includes damage to tumor microvasculature, damage to microvasculature supplying premalignant lesions and tumors, diffusion of heat to tumor cells to damage or kill the same, thermal damage to the urothelium and urothelial cells, and separation of the urothelium, tumor-containing urothelium, or urothelial, dysplastic urothelial, or malignant cells from the basement membrane. Tumors or premalignant lesions subjected to direct photothermal injury may also be more susceptible to further injury with exposure to chemotherapeutic or anticancer agents.

In these model calculations, the spot size of the laser beam on the bladder wall is 3 mm diameter. This is a relatively small spot size, compared the capabilities of laser technology to produce high pulse energies. The 3 mm spot size was assumed herein in order to make the Monte Carlo and heat transfer calculations tractable, when a small grid size of 50 μm is used to accommodate the small blood vessels included in the model. There is no limit to the spot size that can be used in accordance with the invention, however. A human bladder is typically 5 cm in diameter. With a 7 mm spot size, approximately 200 pulses would be required to cover the entire inner surface area. The speed of the laser procedure would be determined by the speed with which the pulses could be directed, and the portion of the entire bladder that is to be treated, however at a pulse rate of 1 Hz, only 3.3 minutes is required to treat an entire bladder. A single pulse, if sufficiently high energy, could treat an entire bladder. With smaller spot sizes, lower pulse energies and pulse repetition rates on the order of 1 or 2 Hz or less, the speed of the procedure remains advantageous.

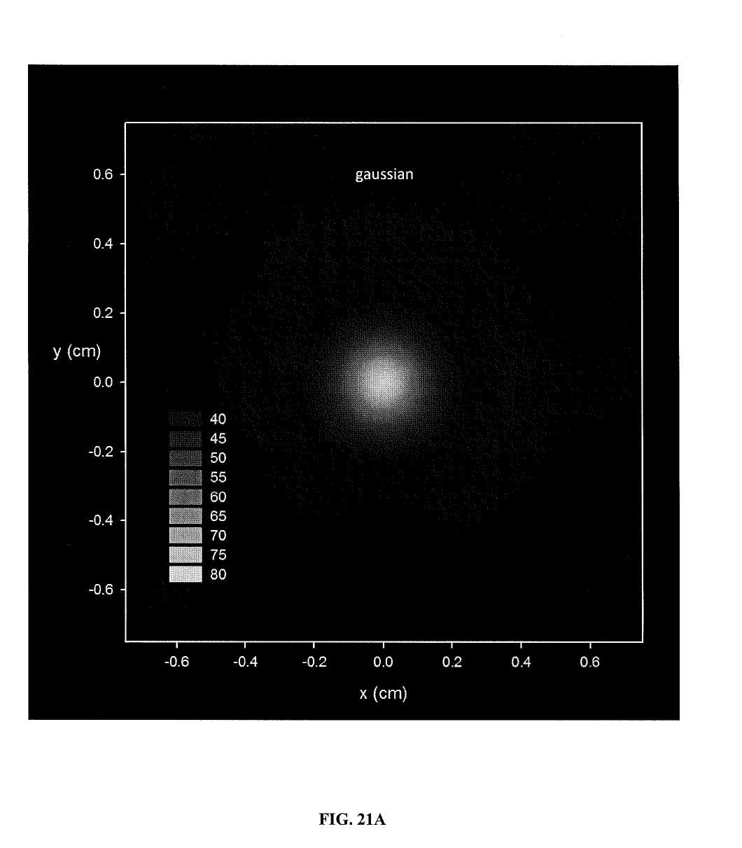

Also in the examples that have been provided herein, a Gaussian beam profile on the tissue was assumed in each case. This profile corresponds to the emission of a flat, cleaved optical fiber held at a distance from the bladder wall, which is one embodiment of the present invention. Alternatively, an optic or optics, for example lenses including gradient-index lenses, can be used to provide other beam profiles, including collimated beam profiles. The distribution of energy on the tissue surface affects the distribution of laser-induced heating. For example, FIGS. 21A and 21B show contour plots of the tissue temperature at the location of the basement membrane (150 μm below the urothelial surface in this model) for Gaussian and collimated 3 mm pulses from a KTP laser, respectively. The collimated spot shows a relatively even energy distribution in the plane of the basement membrane over an area approximately equal to the 3 mm diameter incident spot on the urothelial surface. The temperature at the end of the 15 ms pulse with fluence 15 J/cm² is about 60° C., consistent with relatively mild heating.

With a Gaussian spot, however, heating is concentrated in the central portion of the basement membrane under the incident irradiated area, and reaches about 80° C. That temperature is consistent with significant thermal damage in most cellular tissues, and may correspond, for example, to damage to the hemidesomosomes that link the basal urothelial cells to the lamina lucida of the basement membrane. Urothelial cells themselves may be thermally injured, the connections (adherens-type junctions, desmosomes, hemidesmosomes) between urothelial cells and/or basement membrane may be loosened, and the mucin layer disrupted, with the likelihood of urothelial injury highest at the center of the Gaussian irradiated spot where both the surface and subsurface fluence is maximized.

One important consequence of the above finding is that contiguous or minimally overlapping circular irradiated spots with Gaussian profiles will produce a discontinuous pattern of injury at the urothelium. Inspection of the contour plots in FIGS. 14, 16, 18, and 20 shows that vascular damage at the level of the mucosal plexus occurs over an area with diameter approximately equal to the incident spot size. Thus, with contiguous or minimally overlapping Gaussian spots it is possible to select laser fluences that produce substantially continuous suburothelial damage with substantially discontinuous urothelial damage. According to embodiments of the invention, it is not necessary to damage, disrupt, or desquamate the entire urothelium, or substantial contiguous areas of urothelium, as discontinuous urothelial layer damage is sufficient to allow applied drug to penetrate into the bladder wall.

It is recognized herein that discontinuous urothelial injury also has the important advantage of greatly increasing the re-epithelialization rate, compared to urothelial injury over substantial contiguous areas. Urothelium regenerates by growth and migration of urothelial cells at the edge of an injury, unlike skin, which reepithelializes mainly from epithelial cells associated with skin appendages (hair follicles and sweat glands) in the dermis. The use of a pulsed light source, delivered to the urothelium with an uneven energy distribution such as, but not limited to, the centrally-peaked Gaussian distribution, generates a treatment site that may consist of a urothelial layer zone that is substantially injured, surrounded by a zone that is substantially uninjured or injured to a substantially lesser extent, such that the surrounding uninjured or less injured zone comprises an intact basement membrane with attached viable basal layer urothelial cells, and such that the relative contribution of the injured and uninjured/less injured zones to the total area of the treatment site is a function of the energy distribution and hence controllable.

The substantially less injured or uninjured zone is an immediate source of urothelial layer regeneration for the adjacent injured zone at each treatment site. Because there is a substantially uninjured/less injured zone of urothelium at each treatment site, there can be a sufficient source of viable attached urothelial cells left on the bladder wall after treatment, regardless of whether the treatment consists of directing laser pulses to a portion of the urothelial surface or the entire urothelium of a bladder, when the radiation has a Gaussian or other uneven energy distribution. Alternatively, if the radiation is provided with a substantially even or homogeneous energy distribution over the irradiated spot on the urothelial layer surface, such that the urothelial layer damage is substantially the same at any point within the treatment site, discontinuous urothelial layer injury can be produced by irradiating the bladder wall with non-overlapping spots so that treatment sites are separated by regions of nonirradiated bladder wall.

A bladder treated according to embodiments of the invention tends to heal more rapidly when the urothelial injury is discontinuous, than when the injury is continuous. Consequently, the barrier function of the bladder of a patient treated according to the present invention in the advantageous discontinuous mode of urothelial layer injury is restored more rapidly, and the possible side effects of cystitis, pain and bleeding are minimized. Treatment of the bladder according to the invention with discontinuous injury to the urothelium is advantageous.

Another aspect of the invention is that it is possible to irradiate the bladder wall such that the basement membrane remains in place covering the lamina propria, when suburothelial damage is produced with or without urothelial damage. Conduction of heat from the vessels of the lamina propria can cause damage to desmosomes and hemidesmosomes of the urothelial cell layers, and loosening of the connections between urothelial cells and between urothelial cells and the lamina lucida layer of the basement membrane. The basement membrane is itself an essentially acellular structure, relatively resistant to thermal injury, and may remain attached to the lamina propria after urothelial cells are loosened or detached. The significance of basement membrane retention is that migration of urothelial cells to fill a zone of urothelial damage will be much more rapid with this scaffolding intact. The preservation of a substantially intact basement membrane is advantageous when treating the bladder to produce discontinuous zones of thermal damage. However, it is recognized herein that preservation of the basement membrane may also allow the entire urothelium to be damaged in a substantially continuous manner.

A pulse of radiation from a laser, such as a flashlamp-pumped, solid-state laser, may, on closer inspection of an actual device, be seen as an envelope or macropulse, consisting of many shorter pulses (micropulses). The pulse durations referred to in the examples above may be macropulses. It is possible, according to the invention, to apply more than one macropulse to each treatment site, to produce suburothelial and/or urothelial layer damage. The use of multiple macropulses approximates the use of longer single macropulses, just as a train of micropulses approximates a macropulse, and is therefore a direct extension of the ideas described above with respect to pulse duration and preferential injury to blood vessels of the lamina propria.

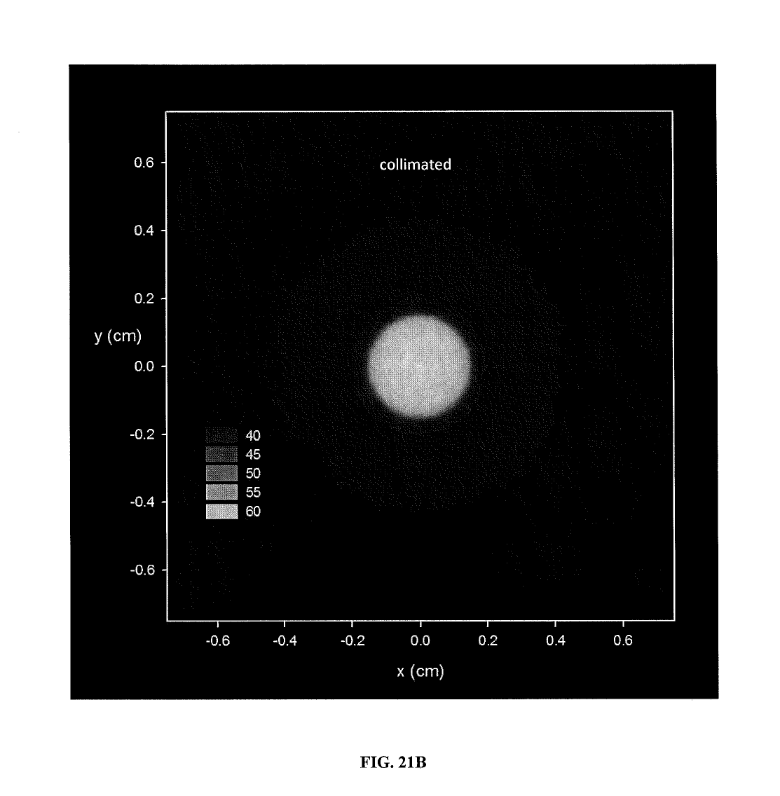

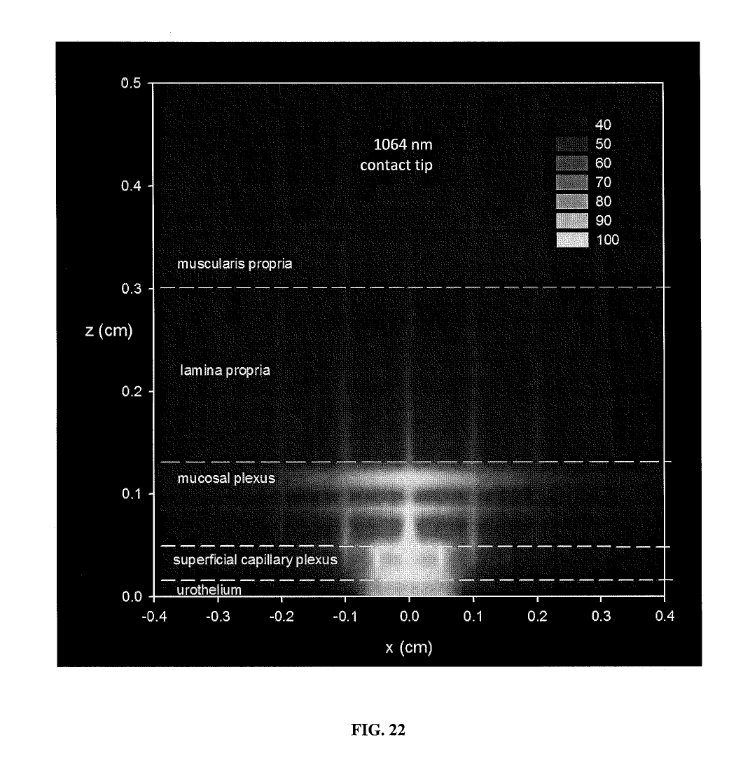

In an alternative embodiment of the invention, the radiation is delivered to the bladder wall using a probe or optic that is in contact with the tissue. FIG. 22 shows results of a calculation performed for irradiation of the bladder wall with a 15 millisecond 1064 nm Nd:YAG laser delivered using a 1.5 mm contact tip and the fully vascularized model. The contact tip produces urothelial temperatures consistent with thermal damage, unlike the same pulsed Nd:YAG laser with noncontact delivery shown in FIG. 20. Many types of contact tips are familiar to those skilled in the art, and include ball tips, shaped tips, and diffusers, made of various materials including but not limited to fused silica and sapphire. Considering the region of bladder wall centered on the probe tip at irradiation, of the area of urothelial layer damage is significantly smaller than the area of suburothelial tissue affected by vascular damage, and the ratio of these areas or zones can be controlled by pulse energy with a pulsed laser.

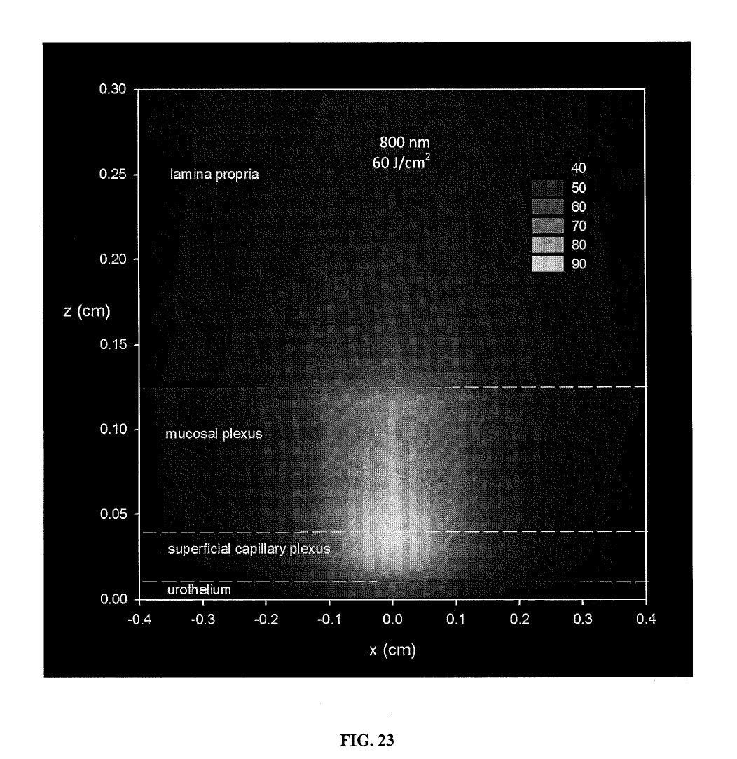

In FIG. 23, the results for the example of a semiconductor diode laser with 40 ms pulse duration are shown. Because the pulse duration is relatively long, heat diffuses from the smaller vessels to a greater extent, although heating remains localized to the lamina propria. The reduced selectivity resulting from longer pulse durations has the relative disadvantage of increasing the possibility of significant injury to nonvascular components of the lamina propria and hence healing with fibrosis, although longer pulses also have the relative advantage of allowing larger vessels to be treated with high energy without rupture. A 40 ms diode laser (e.g., one available from Coherent) operating at approximately 800 nm has been used for hair removal. It is possible to increase the power density of such a laser and decrease its pulse duration to a few milliseconds to produce more selective vascular damage to the lamina propria.

In another embodiment of the invention, the radiation from a laser is delivered to the urothelium of the urethra or one or both ureters, using a probe adapted to be inserted in a ureter or urethra. An advantageous probe is of a side-firing type, or has a contact tip, or both. A side firing probe may be an optical fiber with an attached distal optic such as an angled mirror or prism to displace the direction of the radiation emitted toward the urothelial surface of the ureter or urethra. Side firing probes for laser treatment of soft tissue are known in the art. According to the present invention, the ureters and urethral may be treated with a laser to decrease permeability of the suburothelial tissue of those structures, increase permeability of the urothelial tissue of those structures, or both. Laser treatment to alter permeability may also be performed to have a direct effect on premalignant malignant lesions located within the ureters and/or urethra, to increase cytoxicity of a chemotherapy drug, or for some combination of these effects.

It is known that damage to urothelium generally predisposes the bladder to seeding of new tumors by viable tumor cells dislodged from an existing tumor and distributed in the fluid of the bladder lumen at the time that that existing tumor is resected. Unnecessary damage to urothelium during tumor treatment has been discouraged for this reason in the prior art, although bladder cancer treatments are known to produce such damage and new tumor growth results from tumor seeding after treatment. However, in the treatment of the bladder according to the present invention, tumor seeding as a result of urothelial layer damage is inhibited. Specifically, it is recognized herein that laser treatment that reduces permeability of suburothelial tissue may also be performed to reduce recurrences due to tumor seeding in the bladder, and also in the ureters or urethra.

When blood vessels are injured in the lamina propria, there is a lack of nutritional support and oxygen for viable tumor cells that may be seeded on the overlying and possibly injured urothelium. As the seeded tumor cells are not initially part of a tumor or tissue with its own microvasculature, they have no other source of supply and are therefore inhibited from growing into a tumor. As the seeded tumor cells are on or in the urothelial surface of the bladder, the suburothelial vascular supply they depend on is readily accessible to damage, as has been shown in the examples above for pulsed dye, KTP, pulsed diode, and flashlamp pumped solid state lasers. Rather than to directly kill tumor cells in the bladder lumen, the approach of the present invention is to instead starve an individual viable tumor cell after it has adhered to the bladder wall but before it has grown into a tumor and developed a functional blood supply. The ability of vascular targeting lasers to effectively inhibit the growth of individual adherent cells on urothelium into new tumors by eradicating the vascular support of those cells has not been recognized previously.

In an alternative embodiment of the invention, two or more laser wavelengths are combined, with at least one of the wavelengths being preferentially absorbed by blood and having pulse duration and energy appropriate for photothermal damage of suburothelial blood vessels, and the other wavelength being preferentially and strongly absorbed by water, for directly damaging the urothelium.

Nonlaser or incoherent light sources may be used according the present invention, for example filtered flashlamps with pulsed emission in the visible and near infrared spectral regions. It is more difficult to efficiently collect the radiation from flashlamps into small diameter optical fibers easily inserted in the working channels of flexible cystoscopes however, so for this reason the use of lasers is preferred.

The temperature of the solution or fluid contained within the bladder can be varied, and the fluid can be flowing or stationary in the bladder. Warming the solution, for example to physiologic temperature (37° C.), can be used to influence the effect of treatment by reducing the amount of laser energy required to heat the urothelium.

The method and device of the present invention does not require the use of thermocouples for the monitoring of tissue temperature, since treatment effects may be controlled by laser parameters, including pulse energy. Electromagnetic radiation can be applied through small diameter fibers readily inserted in the working channels of endoscopes including flexible endoscopes. This capability, as well as the minimal pain associated with treatment of tissue with vascular targeting lasers, allows the treatment of the present invention to be performed in a clinic or office with no anesthesia or with only local anesthesia. This ability to perform laser surgery without general or regional anesthesia leads to significant cost savings compared to surgery in an operating room. In addition, a procedure with only local anesthesia is less risky for patients who are elderly or in poor health.

Although the examples of the present invention involve electromagnetic radiation and light in the near infrared and visible regions, it is recognized that other wavelengths and forms of radiation may be useful, including microwave, ultrasound, and radiofrequency.

Theoretical mathematical model calculations have been performed herein using to determine the previously unknown photothermal effect of light on vascularized bladder tissue. Without wishing to be bound by any particular theory, model, or anatomic or optical data, these theoretical calculations show the feasibility of targeting the vasculature of the lamina propria of the bladder, using visible or near-infrared light. The vessels of the lamina propria may be directly injured by absorption of light, and the overlying urothelium may be injured by transfer of heat from the vessels.

With these results, the effect of injury to bladder wall structures can be demonstrated to have effects on drug pharmacokinetics, for specific drugs used in intravesical chemotherapy. Specific examples are provided herein, however the present invention is applicable to any drug or agent that can be applied to tissue.

The pharmacokinetics of drugs given intravesically are described using a distributed model, where both diffusion through the extracellular space of the bladder wall and uptake by blood vessels within the bladder wall occur. Paclitaxel is a taxane used for systemic chemotherapy, and it is presently of interest as a potential intravesical agent for bladder cancer. Aspects of the invention can be exemplified with paclitaxel, showing the effect of laser treatment on drug pharmacokinetics.

The half width ω_{1/2} of drug concentration in the bladder wall is related to tissue properties in the distributed model by the formula ω_{1/2}=0.693(D/pα)^{1/2}, where D is the diffusion coefficient of drug in tissue, p the permeability coefficient of the capillaries, and α the surface area of the capillaries. The half width of 381 μm for paclitaxel implies D/pa=3.02×10⁵ μm² for bladder wall tissue in its untreated, native state.

If the bladder is irradiated before aqueous paclitaxel is instilled using a 585 nm pulsed dye laser with pulse duration 0.5 ms and minimally overlapping spots, blood vessels of the lamina propria will be preferentially heated. Below a threshold fluence for urothelial damage, thermal damage may be substantially limited to the blood vessels in the bladder wall. The threshold fluence for vascular damage may vary from patient to patient or between locations within the bladder, but may be determined in practice by the observation of the minimum fluence required to produce a darkening of the bladder vessels, blanching of the bladder wall, or both. Consequently, there is a range of fluences in which the blood vessels of the lamina propria may be coagulated, but the urothelium may be substantially unaffected. The percentage of blood vessels in the lamina propria that are coagulated will depend on the fluence used, on the proportional area of bladder surface that is irradiated, and on energy distribution (homogeneous or uneven energy distribution). A first-order estimate of the effect of this laser treatment on the pharmacokinetics is made by realizing that the total surface area a of the capillaries (and other vessels such as arterioles and venules of the lamina propria) can be effectively reduced by the laser treatment, since coagulated vessels are impermeable to drug.

If the laser treatment fluence is selected so that the blood vessel surface area is decreased by 50%, D/pa becomes 6.04×10⁵ μm², and ω_{1/2} increases to 538 μm. If the surface area decreased by 75%, ω_{1/2} becomes 761 μm. With a 90% reduction in surface area, ω_{1/2} increases to 1.2 mm. Hence, with a 75% reduction in surface area, the distance over which the concentration in the bladder wall drops by half is doubled over its original value of 381 μm. With a 90% reduction in surface area, the half width corresponds to a large portion of the lamina propria, where invasive tumors are located. The use of the laser according to the present invention therefore has been found to shift the depth of paclitaxel concentration to deeper layers in the bladder wall where more invasive tumors may be located.

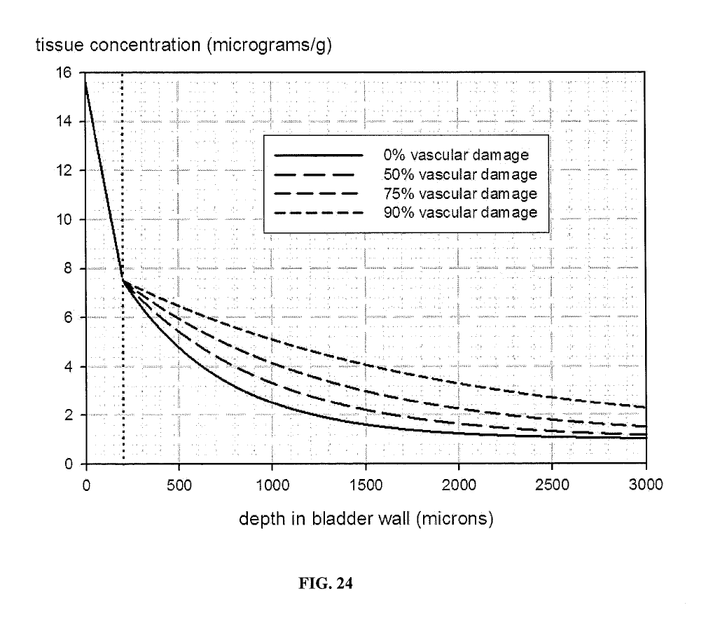

This effect is seen in FIG. 24, where the computational results of this example of the present invention are depicted. Over the thickness of the urothelium (200 μm in this example), the concentration of paclitaxel decreases linearly according to Fick's Law since there are no vessels in that tissue layer. Below the urothelium, the decline is exponential due to both diffusion through extracellular space and uptake by vessels. With laser parameters that have no effect on the urothelium, the kinetics of drug diffusion is unchanged in that layer. However, as can be seen, the drug concentration in suburothelial layers is strongly dependent on the coagulation of blood vessels. The drug concentration at 1 mm below the bladder surface for 0% (untreated), 50%, 75% and 90% reduction in vessels is 2.52, 3.32, 4.14, and 5.10 μg/mg, respectively. These increases in drug concentration within the lamina propria, for a given constant concentration of drug in urine and urothelium, provide a significantly increased exposure of the tissue to the drug in the suburothelial layers.

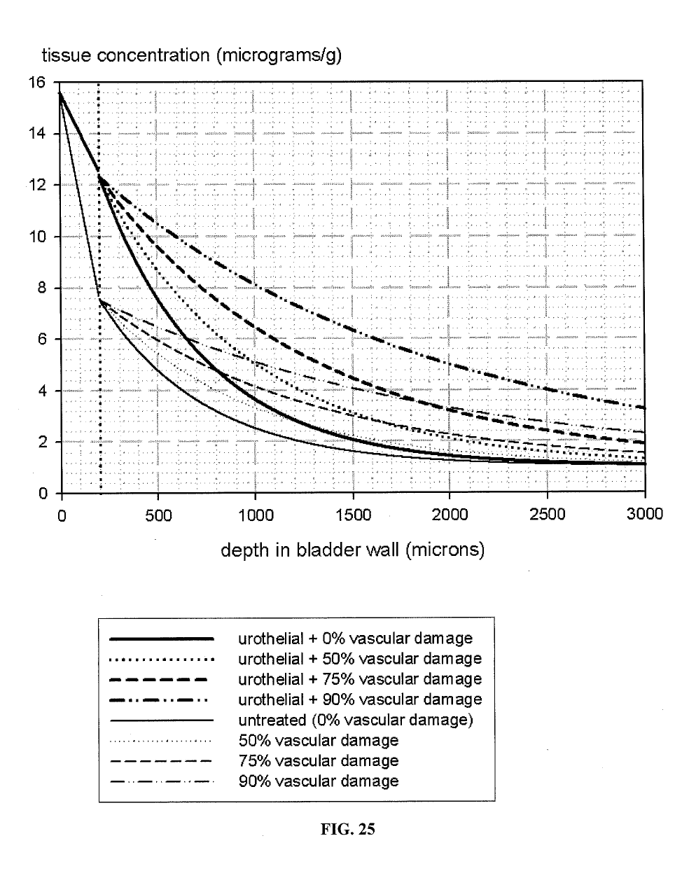

Further increases in drug concentration can be achieved by increasing the permeability of the urothelium, according to the present invention. As another example, the 585 nm pulsed dye laser with 3 mm spot size and 0.5 ms pulse duration may be used at higher fluences, or with multiple superimposed spots, to create a vascular injury with additional urothelial damage. Specifically, the urothelium may be damaged such that it becomes more permeable. The increase in permeability will depend on the amount of damage to the urothelium. Laser parameters corresponding to substantial injury to the urothelium with desquamation of the superficial and intermediate urothelial cell layers can be expected to substantially increase the permeability of the urothelium to drugs. Assuming that the laser treatment increases the permeability of the urothelium to paclitaxel, such that the ratio Cᵤᵣₒ/Cᵤᵣᵢₙₑ increases from its untreated value of 0.48 to a substantially higher value of 0.80, the concentration of paclitaxel in the suburothelial layers of the bladder wall will also further increase. This result is shown in FIG. 25. In this calculation, the concentration of paclitaxel in blood in the deepest layers of the bladder wall with damaged urothelium are assumed to be the same as in the case where the urothelium is undamaged.

In the standard practice of intravesical chemotherapy, the dosage of the drug instilled into the bladder will be adjusted as necessary to keep the blood concentration to an acceptably low level, to avoid systemic toxicity. According to the present invention, this adjustment of dosage will be performed as necessary. Before treating patients with a given choice of drug and laser treatment parameters, it will be necessary to perform the standard required trials to monitor blood plasma levels of the drug, to ensure they remain below toxic levels.

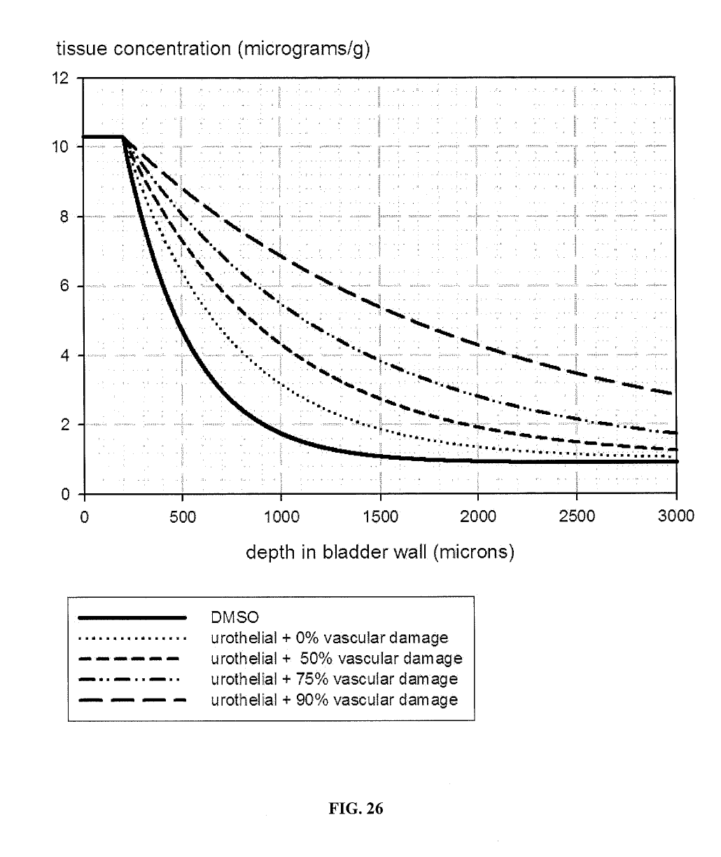

The solid line of FIG. 26 shows the tissue concentration profile for paclitaxel dissolved in 50% DMSO, a penetration enhancer. The urothelial layer concentration of paclitaxel is high but the drug concentration falls off rapidly in the lamina propria. Substantial injury to the urothelium can be produced by a laser according to methods of the invention such that the concentration of paclitaxel from an instilled aqueous solution at the junction between the urothelium and lamina propria (or, equivalently, at the basement membrane, if the urothelial cell layers have been completely lost in this urothelial injury) is the same as the urothelial layer concentration when 50% DMSO is instilled in an bladder not treated by the laser. With various amounts of laser-induced vascular damage in addition to the urothelial layer damage, the concentrations of drug at depth in the tissue is greatly increased over the concentrations achieved with DMSO treatment only, as may be seen from FIG. 26. Thus, the present invention provides substantial advantages over the well known permeability enhancer DMSO.

For the treatment of bladder cancer, MMC is the most commonly used intravesical drug. MMC has very different physicochemical and pharmacokinetic properties from paclitaxel. Because MMC is hydrophilic, it partitions from the urine into the urothelium less than does paclitaxel, by an order of magnitude. The use of MMC according to the present invention is described in the example below.

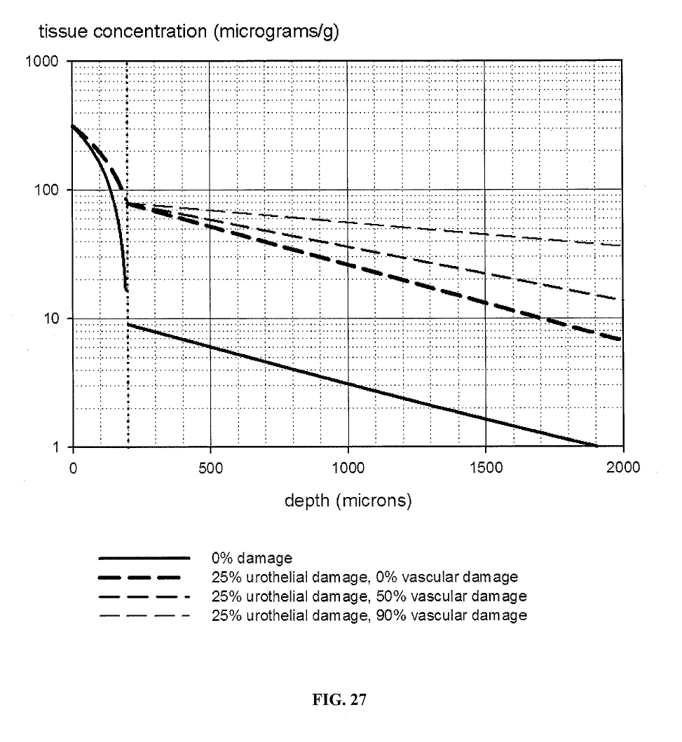

FIG. 27 shows results of a calculation of the tissue concentration versus depth in the bladder wall, for a urine MMC concentration of 315 μg/ml, corresponding to a dose of MMC 5 minutes after administration. The curve for untreated bladder are shown along with curves corresponding to laser-treated bladder. Specifically, the bladder has been treated with a laser to allow permeation of MMC into the urothelium to a level 25% of the urine concentration. Fifty or 90 percent of suburothelial blood vessels are damaged, or the urothelium is damaged with no concomitant vascular damage. Because the untreated urothelium is minimally penetrated by MMC, a log scale is used to represent the concentrations over the bladder wall.

The calculations for MMC and paclitaxel demonstrate the benefits of the present invention for the examples of a very hydrophilic drug and a very lipophilic drug, respectively, and thus cover the range of partition coefficients.

As noted previously, treatment may prevent tumor seeding by inhibiting the implantation of tumor cells, or by inhibiting the growth of implanted cells. By substantially damaging the suburothelial vasculature, the vascular support of the tumor cells is disrupted or eliminated. Also, if a chemotherapeutic drug is administered after the suburothelial vasculature is damaged, growth of seed cells on the urothelium will be inhibited by both direct cytotoxic drug effect on the cell and starvation of the cell due to damage to its vascular support.

Delivery of the laser radiation as modelled here can be achieved in practice using standard endoscopes developed for urology, either rigid or flexible. For example, a flexible cysto-urethroscope (Storz model 11272C) with 37 mm working length, 15.5 Fr sheath size and 7 Fr working channel would easily accommodate an optical fiber. The optical fiber is connected to the source of radiation and passed through the working channel. The optical fiber may have a simple cleaved distal end. The distance of the distal end surface from the bladder wall may be gauged by comparison of the known fiber diameter to the diameter of a low power visible aiming beam provided with the source of radiation. Alternatively, the fiber or endoscope may have an attached tip that comes into contact with the bladder wall at or near the intended irradiation site, and which serves as a distance gauge. In advantageous configurations, this tip or distance gauge has a rounded, soft, deformable, or otherwise atraumatic structure so that it does not significantly damage the bladder wall at the area of contact.

The drug of the present invention can include, without limitation, a chemotherapeutic drug, anticancer agent, antibiotic, antiangiogenic or antiproliferative agent, cytokine, protein, peptide, radionucleotide, dye, photodynamic or photochemotherapeutic agent, bioreductive drug, plasminogen activator inhibitor, anesthetic agent, imaging agent, interferon or immune modulator such as BCG. Furthermore, the drug can be applied in aqueous form or in any other solution, using penetration enhancer, formulation, matrix, or vehicle, including but not limited to polymer, buffer, emulsion, micelle, liposome, mucoadhesive, gel, microparticle, or nanoparticle formulation. The drug may be instilled into the bladder or other hollow organ. For other organs or tissue surfaces the drug may be administered in a form that contacts or adheres to the surface of an organ or tissue, or by using a patch or other localized delivery device including but not limited to iontophoretic, electromotive, electroporation and ultrasonic energy delivery devices.

One aspect of the invention is that photothermal injury to blood vessels in the bladder wall has the concomitant effect of reducing oxygen tension in the treated tissue due to the reduction in blood flow. Reduced oxygenation has the effect of inhibiting the growth of abnormal cells into tumors, and of damaging existing tumors and tumor cells as described above. Reducing the oxygen tension in the treated tissue also has an additional important effect. It is known that many solid tumors in human tissues have regions of hypoxia that are resistant to treatment by ionizing radiation or standard chemotherapy.

Bioreductive drugs have been devised as a method of targeting hypoxic tumor cells, for example by systemic administration in conjunction with radiation that targets the well oxygenated tumor regions. Bioreductive drugs are reduced by certain enzymes in the tissue environment to a more highly cytotoxic metabolite, although oxygen may reverse that activation so that a process termed futile cycling occurs. Depending on the bioreductive drug and the enzyme levels of the tissue, a bioreductive drug may be activated in well-oxygenated tissue or hypoxic tissue, or there can be preferential toxicity in tissues with low oxygen tension. MMC, the most commonly used bladder cancer intravesical chemotherapy agent, is a bioreductive drug. Other chemotherapy drugs already in use or in development for bladder cancer that are bioreductive agents include doxorubicin and eoquin.

According to current usage, the cytoxicity of such drugs in the bladder is controlled by the naturally occurring levels of oxygen and enzymes (including one electron reductase NADPH:cytochrome C (P450) reductase and two-electron reductase DT diaphorase) in normal and tumor tissue (US 2007/0185188). In U.S. Pat. No. 6,240,925, incorporated herein by reference in its entirety, the present inventor describes a method and device for activating a bioreductive agent by inducing hypoxia through photothermal damage to blood vessels, for treatment of cancer. Photothermal vascular damage may create hypoxia in well oxygenated tissue and further increase the level of hypoxia in tissues having naturally occurring low oxygen tension. For bioreductive drugs that have limited efficacy in a particular well-oxygenated or somewhat hypoxic tissue, increased hypoxia induced by photothermal vascular targeting may increase drug activation.

It is recognized herein that not only can the exposure of bladder wall to these drugs be increased by the methods of altered pharmacokinetics of the present invention, but that the cytoxicity and efficacy of the drugs may be enhanced by increased activation or potentiation in the hypoxic environment created by treatment according to the invention. Various other quinones, N-oxides, nitroaromatics, uracils, and cobalt(III) complexes, for example porfiromycin, CI-1010, tirapazamine, 90CE, TX402, NLCQ-1, OFU001, and CTC-96, have been studied as bioreductive drugs and may be useful in accordance with the present invention. In a bladder wall treated according to the present invention so that suburothelial blood verses are damaged and hypoxia is induced, a bioreductive chemotherapeutic drug may be made more active against tumor cells and, at the same time, tumor cells will be exposed to higher levels of the drug.

As a specific example, improvements in the effectiveness of the bioreductive drug eoquin against bladder cancer can be shown with the present invention. Eoquin is reported to be effective against the aerobic fraction of bladder tumor cells having high levels of NQ01 (DT diaphorase), such as low grade tumors (US 2007/0185188). Two electron reductases produce toxic hydroquinones from quinones, in the presence or absence of oxygen. In high grade tumors having low NQ01 levels, but high levels of NADPH:cytochrome C (P450) reductase, eoquin is reduced to a highly toxic free radical semiquinone. However, in the presence of air this semiquinone reacts with oxygen, with reactive oxygen species as the end result. The reactive oxygen species do not have high anticancer activity. It was therefore concluded that eoquin should be combined with radiotherapy or another chemotherapy drug to treat the aerobic fraction of higher grade bladder tumors, such tumors typically having low NQ01 levels (US 2007/0185188).

Photothermal targeting of vessels in the bladder wall for altering bladder wall permeability according to the present invention induces hypoxia or increases the level of hypoxia in high grade tumors, thus increasing the toxicity of eoquin by preventing the back oxidation of the semiquinone. Consequently, high grade bladder tumors are sensitized, so that they may be treated effectively with eoquin, without the need to add ionizing radiation or another drug. Bioreductive activation by the 2 electron mechanism has toxicity for both aerobic and hypoxic cells, which has been a drawback in previous clinical application of eoquin, but damage to normal aerobic tissue such as liver, kidney and intestine that has high NQ01 levels is avoided in treatment according to the present invention by localized delivery of drug to the bladder. Also with the present invention, the exposure of the bladder wall to eoquin and its toxic metabolite is increased due to alterations in bladder wall permeability that increase the concentration of eoquin and/or increase the duration of exposure to eoquin.

It may be appreciated that the present invention introduces new mechanisms for the treatment of bladder cancer, and that the multiple mechanisms in combination work synergistically for a highly effective and improved treatment.

FIGS. 28A and 28B are schematic depictions of one embodiment of the invention. FIG. 28A shows a laser device 20 that includes a user interface display 21, calibration port 23, laser emission port 24, optical fiber 25, fiber pole 26, and footswitch 22. The interface display 21 and footswitch 22 may be used to control laser settings and to actuate the laser device 20. FIG. 28B is a schematic depiction of a flexible cystoscope 30 used to deliver radiation generated by a laser, such as the laser device 20 of FIG. 28B, to a treatment site. The cytoscope 30 includes a laser port 32 configured to receive an optical fiber 25 that carries laser radiation from the laser emission port 24 of the laser device 20. The cytoscope 30 also includes an irrigant port 33, irrigant tubing 37, light port 34, light source fiber 38, and eyepiece 36. The optical fiber 25 attached at its proximal end to the laser emission port 24 is passed through the working channel of the cystoscope 30. The distal end of the laser fiber 25 may be extended beyond the distal tip 31. In other embodiments of the invention, a flexible urethroscope, a rigid cystoscope, video urethroscope, or endoscope suitable for use in the bladder may be used instead of a flexible cystoscope.

Certain devices of the present invention are well suited for adaption to a multiple use system for surgery. For example, the KTP laser of the example of the present invention may be configured to also operate in a quasi-continuous manner with very high pulse repetition rate and low pulse energy, to incise or coagulate tissue for general surgical purposes. A semiconductor diode laser may be used in pulsed mode according to another example herein and may also be used in continuous wave mode to incise or coagulate tissue in a standard surgical manner. Such a multiple-use system that could be used according to treat cancer according to the present invention and also to ablate, excise, or incise bladder tumors, tumors of other types, and noncancerous tissues would be of even greater utility.

It may be appreciated that while the present invention is described in detail for the application of bladder cancer, it may be adapted for other malignant or premalignant conditions. These conditions may effect mucosal and epithelial tissues, and may include cancers of the oral cavity, pharynx, larynx, bronchus, lung, esophagus, stomach, small intestine, colon, other digestive organs, kidney, uterine cervix and uterine corpous, ovary, breast, and skin, and premalignant conditions such as Barrett's esophagus.

A particularly advantageous application of the present invention is the treatment of skin cancer, particularly basal cell carcinoma (BCC). Surgery is the standard treatment for BCC, and even in its most precise and tissue-selective form, Mohs surgery, normal tissue is removed along with tumor, leaving a skin defect that may require reconstructive surgery. Many BCCs, especially low risk superficial tumors, are treated with simple excision, electrodessication and curettage, and cryosurgery. Scarring and hypopigmention result. Chemotherapy has a limited role in treatment of BCC, in part because of barrier function of the epidermis to topically applied substances.

According to the present invention, BCC of the skin may be treated with a laser to selectively injure blood vessels of the tumor and adjacent normal skin tissue, with simultaneous injury to the overlying epithelium, followed by application of a therapeutic anticancer, antiproliferative, or chemotherapeutic agent to the treatment site. The invention may be implemented using a standard commercial vascular lesion laser such as the Vbeam (Candela) or VStar (Cynosure) pulsed dye lasers, or the Viridis (Quantel) and Aura (Ifidex) KTP lasers. For example, these standard lasers may be used at a laser fluence and with no skin cooling or low cooling, so that epidermal injury occurs along with vascular injury to the blood vessels of the underlying dermis. Alternatively, the lasers may be modified to produce a Gaussian beam profile, such that epidermal injury occurs only at a central area within the treatment spot. The laser-induced epidermal injury over all or part of the treated area will weaken or eliminate the barrier function of the epidermis by damaging epidermal cells and causing the cells to separate from each other and at the basal layer. As a result, a topical substance, applied before or soon after laser treatment, will be able to penetrate to the tumor cells located within the epidermis and/or dermis.

Because it is necessary to be able to control the amount of topical substance penetrating into the tumor location, thereby assuring a therapeutic but not overly high dosage to tumor cells, an advantageous implementation of the present invention is to provide an additional means of controlling the amount and location of epidermal injury. It is also advantageous to provide a means of controlling the amount and location of epidermal injury independently of laser fluence, so that a laser fluence can be chosen on the basis of optimal thermal injury to tumor vasculature. Furthermore, it is advantageous to provide a means of controlling the amount and location of epidermal injury such that substantially uninjured epidermis is distributed over the treatment site. Consequently, healing of the epidermis after laser treatment, which occurs from the uninjured epidermis, is more rapid.

It is useful to implement the invention in a manner that produces an advantageously high or maximal concentration of drug in the dermis. For instance, in treatment of BCC and other tumors of the skin, it may be advantageous to expose the skin tumor to a high level of a topically applied anticancer or chemotherapeutic drug, for improved efficacy in killing the skin tumor cells.

According to this aspect of the invention, it is advantageous to substantially maximize the concentration of drug at deep tissue layers where tumor cells may reside. The well known chemotherapeutic drugs MMC and 5-fluorouracil (5FU) are employed in the present model calculations. MMC is a large amphiphilic drug, and 5FU is small and hydrophilic, therefore this example demonstrates the usefulness of the present invention for any drug or chemical substance that can be applied to the skin.

The present invention is illustrated using model calculations of skin pharmacokinetics. First, the concentrations of MMC and 5FU as a function of depth in dermis are calculated assuming that the drug is applied as a pH 7.4 solution to an area of the skin with the viable epidermis removed. The calculation is done for the case of skin with no vascular injury, and for skin that has been treated with a laser or other source of electromagnetic radiation to produce photothermal vascular injury as has been previously described herein. The calculation illustrates how the novel approach of the present invention can achieve the objective of high drug concentration in deep tissue layers.

The dermis is a semi-solid layer with structural collagen fibers disposed within a fluid medium, with microvessels arranged throughout. The time-dependent permeation of a drug through this tissue layer is modeled using the equation below, where C is the drug concentration at depth z and time t, D is the diffusion coefficient for the drug in dermis, and k_{cl} is the rate constant for clearance of the drug from the tissue via uptake by blood vessels:

$$\frac{\partial }{\partial t} C ( t , z ) = D \frac{{\partial }^{2}}{\partial {z}^{2}} C ( t , z ) - {k}_{cl} C ( t , z ) . \qquad ( 1 )$$

With boundary conditions C(t≧0, z=0)=C₀, C(0, z)=0, and C(t>0, z=∞)=0, where C₀ is the concentration of the topical drug on the tissue surface (z=0), the solution to Eq. 1 is:

$$\frac{C ( t , z )}{{C}_{0}} = \frac{1}{2} exp ( - z \sqrt{\frac{{k}_{cl}}{D}} ) \cdot erfc [ \frac{z}{2 \sqrt{tD}} - \sqrt{{k}_{cl} t} ] + \frac{1}{2} exp ( z \sqrt{\frac{{k}_{cl}}{D}} ) \cdot erfc [ \frac{z}{2 \sqrt{tD}} + \sqrt{{k}_{cl} t} ] . \qquad ( 2 )$$

When k_{cl} t is larger than about 4, Eq. 2 reduces to the steady state form:

$$\frac{C ( z )}{{C}_{0}} ≅ exp ( - z \sqrt{\frac{{k}_{cl}}{D}} ) . \qquad ( 3 )$$

In the absence of a clearance term (k_{cl}→0), the solution to Eq. 1 is:

$$\frac{C ( t , z )}{{C}_{0}} ≅ erfc ( \frac{z}{2 \sqrt{Dt}} ) . \qquad ( 4 )$$

The thickness of tissue over which the concentration of a specific drug declines by half is related at steady state to the pharmacokinetic parameters D and k_{cl} by the expression:

w_{1/2}=0.693\sqrt{D/k_{cl}}, (5)

To calculate the in vivo disposition of a drug applied directly to the upper dermis, Eqs. 2 and 4 are used with D and k_{cl} values derived from the human serum albumin (HSA) binding constant K_{b}, octanol water partition coefficient K_{oct} at pH 7.4, Stokes radius, and molar volume V (Table 1).

TABLE 1
Properties of 5FU and MMC.

| | MW | pKa | K_{b} | K_{oct} | rₛ | V |
|---|---|---|---|---|---|---|
| 5FU | 130.08 | 7.93 | 3.65 × 10³ | 0.129 | 2.26 Å | 101.5 cm³ |
| | | (ref. 12) | M⁻¹ | (ref. | (ref. | mol⁻¹ |
| | | | (ref. 14) | 16) | 17) | |
| MMC | 334.33 | −1.2, 2.7, | 2.07 × 10⁴ | 0.417 | — | 322.0 cm³ |
| | | 7.6 | M⁻¹ | (ref. 16) | | mol⁻¹ |
| | | (ref. 13) | (ref. 15) | | | |

The diffusivity of a drug in dermis is:

D=D_{aq}/3.7, (6)

with the aqueous diffusivity obtained using the Wilke-Chang correlation:

D_{aq}=4.72×10⁻⁷T/μV^{0.6}, (7)

where T is temperature in K, μ is the viscosity of water at T in cP, V is the molar volume of the drug in cm³ mol⁻¹, and the resultant D_{aq} value is in cm² s⁻¹.

The rate at which drug is taken up by the blood vessels distributed in the dermis is taken as the product of the permeability and the surface area of those microvessels:

k_{cl}=P_{cap}·S (8)

Herein, S is calculated in terms of the capillary volume fraction f_{cap} and capillary radius r_{cap}:

$${k}_{cl} = ( \frac{2}{{r}_{cap}} ) ( \frac{{P}_{cap} {f}_{cap}}{1 - {f}_{cap}} ) \qquad ( 9 )$$

The value of f_{cap} for papillary dermis in human forearm skin, 0.0198, is used. Most blood vessels in the human papillary dermis have diameters in the 17 to 22 μm range, therefore 10 μm is taken as a representative value for the radius r_{cap} of a dermal capillary.

The remaining parameter required, P_{cap}, is estimated for 5FU and MMC from cutaneous capillary permeability in combination with a pore model of microvascular permeability. According to the pore model, in which drug passively diffuses into cylindrical pores in the capillary wall, permeability is given by:

$${P}_{cap} = \frac{N π {R}^{2}}{L} {D}_{pore} {ϕ}_{pore} \qquad ( 10 )$$

where N is the number of pores per unit surface area of the capillary wall, R is the capillary pore radius, L the capillary wall thickness, and

φₚₒᵣₑ=(1−α)², (11)

Dₚₒᵣₑ=D_{aq[}1−2.10444α+2.08877α³−0.094813α⁵−1.372α⁶], (12)

with α=r_{S}/R, where r_{S} is the Stokes radius of the drug. r_{S}=4.39 Å and D_{aq}=7.4×10⁻⁶ at 37° C. The pore radius for non-fenestrated cutaneous capillaries is about 5 nm. P_{cap} for 5FU and MMC are determined from their respective Stokes radii and aqueous diffusivities as calculated from Eq. 7. For MMC, r_{S} is taken to be 4 Å.

The effect of vascular injury is included in the model by reducing the value of the capillary volume fraction f_{cap} in Eq. 9 by amounts that can be produced by selective photothermal damage. Table 2 shows the results of these calculations for the diffusion coefficient of MMC and 5FU in dermis, and the respective capillary permeability coefficients, vascular clearance rates, and depth in dermis at which the concentration falls to half of the surface value when steady state has been achieved. w_{1/2} is used herein to describe drug penetration depth in both steady state and non-steady state conditions.

TABLE 2
Pharmacokinetic parameters calculated for 5FU and MMC in dermis
at physiologic temperature.

| values at 37° C. | 5FU | MMC |
|---|---|---|
| D_{aq} (cm² s⁻¹) | 1.36 × 10⁻⁵ | 6.80 × 10⁻⁶ |
| D (cm² s⁻¹) | 3.67 × 10⁻⁶ | 1.84 × 10⁻⁶ |
| P_{cap} (cm s⁻¹) | 2.01 × 10⁻⁵ | 8.58 × 10⁻⁶ |
| k_{cl} (s⁻¹) | 8.11 × 10⁻⁴ | 3.47 × 10⁻⁴ |
| w_{1/2} (μm) at steady state | 466 | 505 |

Despite the differences in the properties of the two drugs MMC and 5FU, the calculations of this example predict relatively little difference in the concentration profiles at steady state, when the blood vessels are normal and undamaged by photothermal treatment. This is a result of the direct dependence of both k_{cl} (rate of uptake by diffusion through aqueous pores in blood vessel walls) and D (rate of diffusion through the aqueous fluid space of the dermis) on the aqueous diffusion coefficient D_{aq}. The concentration of both drugs falls off exponentially with depth in tissue, and the concentration is less than 50% of the surface concentration only about 500 microns below the surface. This calculation provides an explanation for the well known clinical finding that chemotherapeutic drugs, as applied topically to the skin in the prior art, for a period of time on the order of several minutes or so, have inadequate efficacy on skin cancer even if epidermis overlying the tumor has been disrupted or removed.

However, a novel finding of the present calculation is that the time to steady state for the drugs is much longer than several minutes, and furthermore, that the time to steady state increases with vascular injury. The time to steady state varies significantly between MMC and 5FU (Table 3), but in either case is on the order of hours. As the percentage of vasculature that is photothermally damaged increases to 75%, the time to steady state and the depth w_{1/2} (μm) at which drug concentration falls to 50% of surface value at steady state both increase substantially, for both MMC and 5FU. For both drugs, the drug penetration depth w_{1/2}(μm) approximately doubles when the drug is applied to skin with 75% vascular damage, compared to skin with normal, undamaged vasculature. Calculations are not performed for 100% photothermal vascular injury, as that amount of damage would correspond to widespread ischemic necrosis of the tissue and a likely undesirable treatment outcome. At steady state, the concentration of drug in dermis will be at its maximum value, therefore applying the drug for a period of time approximately equal to or greater than the time to steady state will, according to the present invention, provides a highly advantageous effective treatment. Inducing vascular damage will increases time to steady state, and the depth of penetration of the drug beyond what is possible in the prior art.

TABLE 3
Calculated values for depth of MMC and 5FU penetration as a function
of vascular damage.

| | values at 37° C. | | | | | | | |
|---|---|---|---|---|---|---|---|---|
| | 5FU | | | | MMC | | | |
| | percentage vascular damage | | | | | | | |
| | 0 | 25 | 50 | 75 | 0 | 25 | 50 | 75 |
| time to | 1.4 | 1.8 | 2.8 | 5.6 | 3.2 | 4.3 | 6.4 | 13.0 |
| steady state | | | | | | | | |
| (hrs) | | | | | | | | |
| w_{1/2} (μm) at | 466 | 539 | 662 | 939 | 505 | 584 | 714 | 1017 |
| steady state | | | | | | | | |

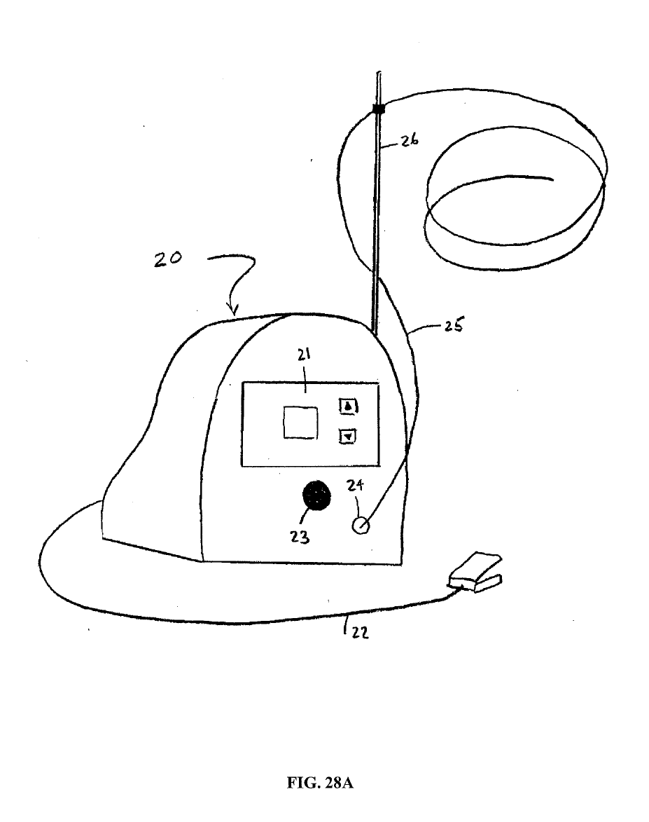

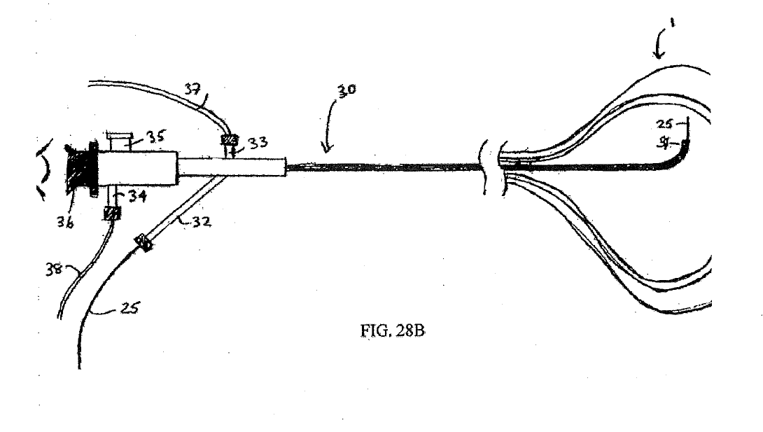

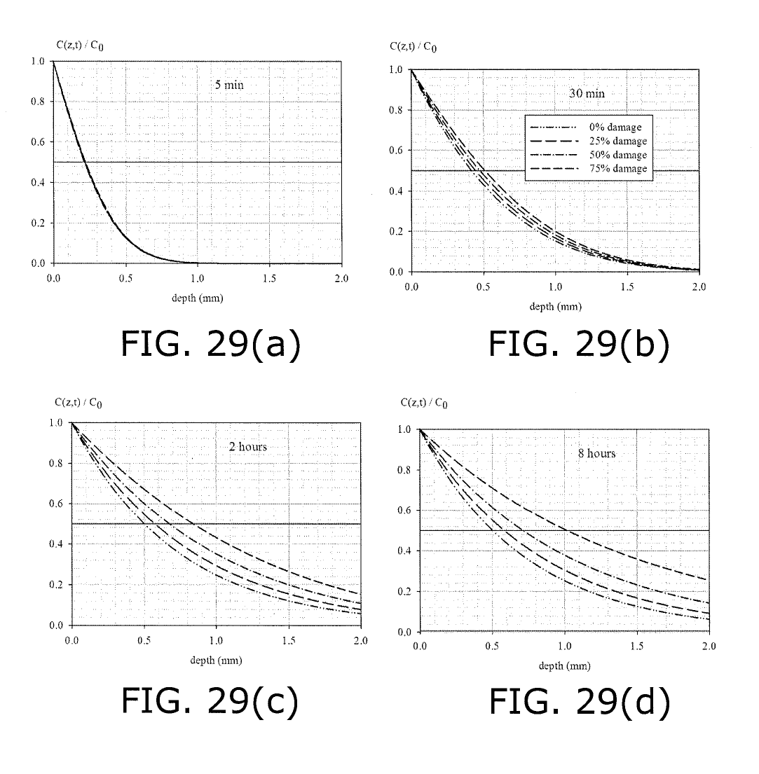

The remaining calculations of this example focus on details of MMC pharmacokinetics. In FIGS. 29((a)-(d)), the concentration profiles for MMC are shown for dermis with distributed microvessels, as calculated using Eq. 2 and the parameters of Table 2. Photothermal vascular injury equivalent to damage to 75%, 50%, 25% and 0% of capillaries is modelled. With an application time of 5 minutes (FIG. 29(a)) there is no difference in drug profile with vascular injury. At this early time period the drug has only begun to diffuse into the dermis, and vascular uptake is insignificant. At 30 minutes (FIG. 29(b)), drug diffusion has increased, and the effect of vasculature is small but observable. MMC concentration falls to half its surface value at a depth (w_{1/2}) of 400 to 500 μm. At the clearance rates k_{cl} corresponding to capillaries with injury of 0 to 75%, 30 minutes is well below the steady state limit. At 2 hours, drug concentration has increased at all depths for all levels of vascular injury (FIG. 29(c)). w_{1/2} is about 500 μm in the absence of vascular injury, but increases to 850 μm for 75% injury. It is apparent that w_{1/2} increases nonlinearly with the fraction of vascular damage. At an application time of 8 hours, the drug concentration for the case of 50 and 75% vascular injury has continued to increase over the 2 hour levels. At a 75% damage level, w_{1/2} is over 1 mm. The concentration profiles for all four damage levels at 14 hours (data not shown) are the same as for 8 hours, indicating that apparent time to steady state is reached earlier than the time calculated using the rigorous definition of 4/k_{cl}. FIG. 29(d) depicts the deepest distribution of MMC that can be achieved in dermis with and without vascular injury. Clearly, with vascular injury, there is a very substantial increase in MMC concentration that can be achieved at deeper tissue depths when the MMC application time is several hours, that is, when the application time approaches the time to steady state for this drug.

Therefore, an important finding of the model calculation described here is that by producing damage to the blood vessels of the tissue on the order of about 25 to 75%, and by applying a topical drug for a length of time that is approximately equal to time required to achieve a steady state concentration of the drug in the dermis, then that concentration of the drug in the tissue, and particularly that concentration of the drug at depth in the tissue, is substantially increased and made more effective than the methods of the prior art. Furthermore, the time required for steady state is substantially greater than the application times of MMC, 5FU and other such topical chemotherapeutic drugs in the prior art.

The clinical significance of the present finding of the invention can be further illustrated by considering both the concentration of the drug in the tissue, and time. The relationship between a defined tissue response x and concentration C(t, z) over total application time tₜₒₜₐₗ is expressed as:

x=∫₀^{t}^{ₜₒₜₐₗ}C(t,z)ⁿdt (13)

where n is a constant that depends on both tissue type and drug. Thus, the effect of drug on tissue depends on both concentration and time, and in the case of a topically applied drug the concentration varies with both depth and, until steady state is reached, with time. A commonly employed tissue response is the percentage of cells that are killed or inhibited. For tumor cells, the drug exposure required for 50% or 90% inhibition of cells (IC₅₀ and IC₉₀) is often used.

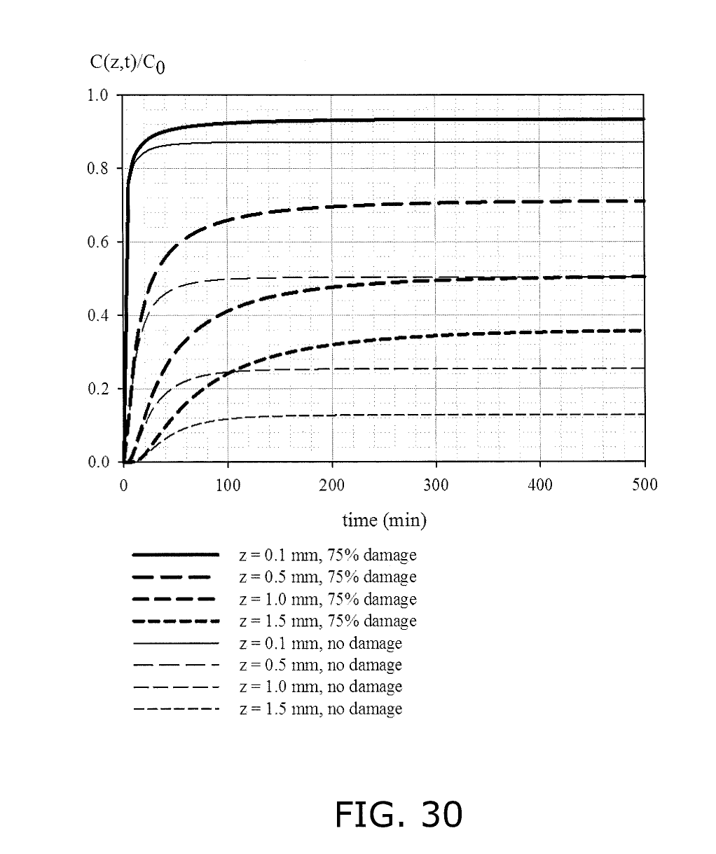

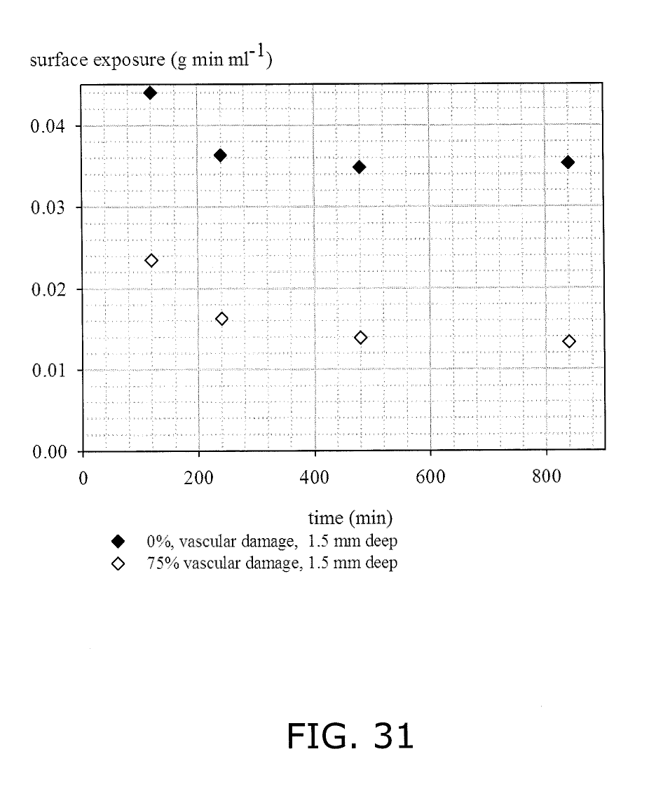

FIG. 30 shows the concentration of MMC as a function of time, at four representative points under the tissue surface (z=0.1, 0.5, 1.0, and 1.5 mm). Using Eq. 13, the total exposure at the subsurface points can be calculated. Because the application of the drug must be tolerable by normal skin tissue as well as be effective in inhibiting or killing tumor cells, the surface exposure to the drug is calculated when exposure at depth is equal to the IC₉₀ exposure for SCC cells. Results are shown in FIG. 31, for 0% and 75% vascular damage, at 30 min, 2 hrs, 4 hrs, 8 hrs, and 14 hrs, at a depth of 1.5 mm. In the absence of vascular damage, the surface exposures required to achieve 90% tumor cell inhibition are high and unchanged between the 4 hrs and longer total application times. Thus, in the absence of vascular damage, long application times do not provide benefit for treatment of tumors located deep in the dermis, and unwanted normal tissue injury can occur, consistent with prior art use of topical chemotherapy agents for treatment of skin tumors. With vascular injury, however, such as may be produced by photothermal laser treatment, the surface exposures continue to drop even after 8 hrs application time. At 75% vascular injury, to produce an IC₉₀ effect at 1.5 mm depth in dermis with 8 hour drug application time, the topical MMC exposure is substantially reduced. Thus, according to the present invention, an large and highly advantageous increase in chemotherapeutic drug concentration at depth in the tissue can be achieved by applying the drug for a time period on the order of hours, to skin that has been subjected to photothermal treatment producing vascular injury, and iatrogenic injury to the skin surface can be avoided.

According to the invention, the viable epidermis can be removed in its entirety or partially, for example with fractional ablations as described previously herein. Removal, ablation, or damage to a portion of the epidermis or stratum corneum of the epidermis will allow the drug to penetrate more readily into the dermis, and may allow faster healing than complete removal. The topical drug or agent can then be applied to the site of epidermal damage for a period of time of at least an hour, or more advantageously of at least 4 hours, or more advantageously yet of at least 8 hours, or until steady state concentration is achieved in the dermis.

It may be noted that in clinical laser treatments, laser irradiation of tissue including skin may have an acute effect of increasing blood flow. This effect may be seen as redness and erythema at the treatment site and nearby. Temporarily increased blood flow may be observed even with irradiation with lasers or other light sources designed to produce photothermal vascular injury. The increased blood flow can be quantified with Doppler blood flow imagers (for example moorLDI2 Laser Doppler Imager, Moor Instruments, Wilmington Conn.), erythema meters (Mexameter® MX 18, Courage+Khazaka electronic GmbH, Germany), for example. To reduce vascular uptake of a topical drug, and thereby reduce unnecessary systemic absorption and or waste of the drug, it may be advantageous to measure the blood flow at the treatment site after irradiation, and wait until the blood flow has dropped below its pretreatment value, indicating that any acute increase has subsided.

As has been described, an objective of the invention is to decrease skin permeability by photothermal injury to blood vessels, thereby increasing the concentration of drug in the skin. As has been shown in the model calculations, the drug concentration profile depends on the amount of vascular injury, as defined for example by the percentage of vessels that are photothermally damaged or the percent reduction in blood flow. In implementation of the present invention, it is advantageous to measure the blood flow at the treatment site after photothermal irradiation, to quantify the amount of damage or blood flow reduction. If the blood flow reduction is at least 10%, more advantageously at least 25%, or more advantageously yet at least 40%, the application of drug may be made. If the blood flow reduction is below the advantageous value, the photothermal treatment can be repeated to achieve an advantageous reduction before the drug is applied to the treatment site.

While chemotherapeutic drugs MMC and 5FU have been discussed in some detail here, the invention is not limited to drugs for the treatment of cancer. Any topical agent, chemical species or drug that is intended to penetrate tissue can be used. Also, the present invention is not limited to treatment of skin, but includes treatment of any tissue in the body to which a drug, therapeutic agent or chemical may be applied.

An example of the present invention is as follows:

A 5 mm surface diameter nodular BCC is diagnosed. The baseline blood flow at the tumor site is measured using Doppler blood flow analysis (moorLDI2 Laser Doppler Imager, Moor Instruments, Wilmington Conn.). The patient's tumor is then treated with a 1064 nm Nd:YAG with 2 ms pulse duration, using an applicator configured as in FIG. 4A, with irradiated spot size 5 mm. Spots are overlapped as necessary to treat the lesion and a 5 mm peri-lesional zone. The sapphire ablation element has light-absorbing elements in the form of 2 mm long carbon-coated needles that are 150 microns in diameter and spaced 0.5 mm apart in a cubic arrangement. A laser fluence of 80 J/cm² or a fluence that produces purpura is used, with the ablation element maintained at 4° C. when treating the surrounding normal skin. After irradiation, blood flow at the treatment site is measured using a Doppler flowmeter, until the blood flow is reduced by at least 25% compared to the baseline value. 5FU cream (Efudex®. Costa Mesa, Calif.: Valeant Pharmaceuticals North America) is then applied to the treatment site, and a bandage applied. The bandage is left on the treatment site for 8 hours or overnight. The bandage is then removed, and the tumor site thoroughly washed to remove remaining 5FU cream. The patient returns for followup examination 4-5 weeks later, and is retreated using the same parameters, if necessary.

While this invention has been particularly shown and described with references to example embodiments thereof, it will be understood by those skilled in the art that various changes in form and details may be made therein without departing from the scope of the invention encompassed by the appended claims.

What is claimed is:

1. An apparatus for modifying a tissue for administration of a therapeutic agent to the tissue, comprising:
a source configured to generate radiation selected to reduce permeability within the tissue to the therapeutic agent; and
an applicator coupled to the source and configured to apply radiation from the source to the treatment site.

2. The apparatus of claim 1, wherein the source is further configured to generate radiation selected to cause photothermal injury to blood vessels of a subsurface layer and to a surface layer of the tissue, the photothermal injury increasing exposure of the subsurface layer to the therapeutic agent.

3. The apparatus of claim 1, wherein the source includes a member of the group consisting of a pulsed laser, a continuous wave laser, and a scanned laser.

4. The apparatus of claim 3, wherein the source is further selected from the group consisting of a KTP laser, a dye laser, a neodymium YAG laser, an alexandrite laser, a semiconductor diode laser, and a fiber laser.

5. The apparatus of claim 1, wherein the source includes a member of the group consisting of a continuous wave incoherent source and a pulsed incoherent source.

6. The apparatus of claim 1, wherein the source is capable of generating radiation at a wavelength of between about 400 nm and about 1100 nm.

7. The apparatus of claim 1, wherein the source is capable of generating radiation with a pulse duration of between about 300 ns and about 100 ms.

8. The apparatus of claim 7, wherein the source is capable of generating radiation at an energy density of between about 3 J/cm² and about 80 J/cm².

9. The apparatus of claim 1, wherein the tissue includes a component of at least one member of a group consisting of a bladder, a ureter, and skin.

10. The apparatus of claim 1, wherein the applicator is configured to apply radiation to an epithelial layer and an upper subepithelial layer and to avoid or prevent damage to a lower subepithelial layer.

11. The apparatus of claim 10, wherein the epithelial layer includes urothelium, the upper subepithelial layer includes lamina propria, and the deeper subepithelial layer includes muscularis propria.

12. The apparatus of claim 1, wherein the therapeutic agent includes at least one member from a group consisting of a chemotherapeutic drug, an anticancer drug, and a bioreductive drug.

13. The apparatus of claim 1, wherein the applicator includes:
an ablation element, the ablation element having a tissue-contacting surface; and
at least one light-absorbing element embedded in, attached to, or coating the tissue-contacting surface of the ablation element.

14. The apparatus of claim 13, wherein the at least one light-absorbing element includes an element from the group consisting of carbon, pyrolytic carbon, iron oxide, and other pigments.

15. An apparatus for the treatment of a tissue, comprising:
a light source;
an applicator coupled to the light source and configured to deliver light emitted by the light source to a surface of the tissue via an ablation element, the ablation element having a tissue-contacting surface and a back surface; and
at least one light-absorbing element embedded in, attached to, or disposed over the tissue-contacting surface or the back surface of the ablation element.

16. The apparatus of claim 15, wherein the light source is configured to emit light comprising at least one wavelength preferentially absorbed by blood.

17. The apparatus of claim 15, wherein the tissue includes skin tissue.

18. The apparatus of claim 15, further including a cooling element configured to cool the tissue, the ablation element, or both the tissue and the ablation element.

19. The apparatus of claim 15, wherein light impinging on a back surface of the ablation element is substantially uniform in energy density.

20. The apparatus of claim 15, wherein the tissue-contacting surface of the ablation element has a diameter of between about 3 mm and about 20 mm.

21. The apparatus of claim 15, wherein the ablation element includes an element from the group consisting of a window and a lens.

22. The apparatus of claim 15, wherein the at least one light-absorbing element includes at least one chromophore.

23. The apparatus of claim 15, wherein the at least one light-absorbing element is selected from the group consisting of carbon, pyrolytic carbon, iron oxide, and other pigments.

24. The apparatus of claim 15, wherein the at least one light-absorbing element includes a member of the group consisting of a layer, a film, and a coating.

25. The apparatus of claim 15, wherein the at least one light-absorbing element has a spatially varying thickness.

26. The apparatus of claim 15, wherein the at least one light-absorbing element includes plural light-absorbing elements arranged in an array disposed over or parallel to the tissue-contacting surface or the back surface of the ablation element.

27. The apparatus of claim 26, wherein the array is a nonuniform array.

28. The apparatus of claim 26, wherein the array is a uniform array.

29. A method of modifying a tissue of a mammalian body, comprising:
generating radiation;
conveying the radiation to a treatment site of the tissue; and
reducing permeability of the tissue to a therapeutic agent by applying the radiation to the treatment site, application of the radiation causing thermal injury to blood vessels at the treatment site.

30. The method of claim 29, further including applying the therapeutic agent to the tissue during or after application of the radiation, the thermal injury increasing exposure time of the tissue to the therapeutic agent.

31. The method of claim 29, wherein the tissue is a multilayered tissue that includes an epithelial layer and an upper subepithelial layer.

32. The method of claim 29, wherein reducing permeability of the tissue includes causing photothermal injury to blood vessels of the upper subepithelial layer, the photothermal injury being sufficient to reduce blood flow in the upper subepithelial layer such that exposure of the upper subepithelial layer to the therapeutic agent is increased during application of the therapeutic agent.

33. The method of claim 32, wherein the multilayered tissue includes a deeper subepithelial layer, and wherein reducing permeability of the tissue includes avoiding or preventing damage to the deeper subepithelial layer.

34. The method of claim 33, wherein the epithelial layer includes urothelium, the upper subepithelial layer includes lamina propria, and the deeper subepithelial layer includes muscularis propria.

35. The method of claim 29, wherein the epithelial layer includes epithelial cells, and wherein reducing permeability of the tissue includes at least one member of the group consisting of: thermally injuring epithelial cells, loosening connections between adjacent epithelial cells, loosening connections between basal epithelial cells and a basement membrane of the tissue, damaging a mucin layer of an epithelium of the tissue, and detaching epithelial cells.

36. The method of claim 35, wherein application of radiation leaves the basement membrane substantially intact.

37. The method of claim 35, wherein reducing permeability of the tissue includes producing a discontinous injury to the epithelial layer, the discontinous injury including multiple zones of thermally injured epithelial cells.

38. The method of claim 37, wherein at least one of the multiple zones has an area of less than about 1 cm².

39. The method of claim 37, wherein at least one of the multiple zones has an area of less than about 0.1 cm².

40. The method of claim 29, wherein reducing permeability of the tissue includes inhibiting growth of tumor cells seeded on or in a surface of the treatment site.

41. The method of claim 29, wherein the tissue includes a component of at least one member of a group consisting of a bladder, a ureter, and skin.

42. The method of claim 29, wherein the therapeutic agent includes at least one member of the group consisting of a chemotherapeutic drug, anticancer drug, and a bioreductive drug.

43. The method of claim 29, wherein generating the radiation includes generating radiation at a wavelength between about 400 nm and about 1100 nm.

44. The method of claim 29, wherein generating the radiation includes generating radiation at an energy density of between about 3 J/cm² and about 80 J/cm².

45. A method of inhibiting tumor growth in tissue, comprising:
generating radiation;
conveying the radiation to a treatment site of the tissue; and
inhibiting growth of tumor cells seeded on or in a surface of the tissue by applying the radiation to the treatment site, application of radiation causing thermal injury to blood vessels at the treatment site.

46. A method of treating a multilayered tissue of a mammalian body, the multilayered tissue including an upper layer and a lower layer, the method comprising:
generating radiation;
conveying the radiation to a treatment site of the tissue; and
reducing blood flow in the lower layer by causing photothermal injury to blood vessels of the lower layer through application of the radiation to the tissue, reduction in blood flow preventing growth of living tumor cells implanted on or attached to the upper layer.

47. A method for treatment of a tissue, comprising:
placing a distal surface of an applicator in contact with a tissue surface, the distal surface including one or more chromophore elements, and the applicator configured to direct light from a light source via the distal surface to the tissue surface;
generating a pulse of light with the light source, the pulse of light being absorbed by the one or more chromophore elements and by blood vessels in the tissue under the distal surface, such that at least a portion of the tissue surface is ablated or removed, and such that a portion of the blood vessels under the tissue surface are coagulated;
removing the applicator from the tissue surface; and
applying a therapeutic substance to tissue surface.

48. The method of claim 47, wherein the tissue includes skin tissue.

49. The method of claim 47, wherein coagulation of the portion of the blood vessels under the tissue surface is sufficient to substantially reduce the permeability of the tissue surrounding the blood vessels to the therapeutic substance.

50. The method of claim 47, wherein the ablation or removal of the portion of the tissue surface is sufficient to substantially increase the permeability of the tissue surface layer to the therapeutic substance.

51. A method for treatment of tissue, comprising:
placing a light delivery element adjacent to a tissue surface of the tissue;
initiating a pulse of light from a light source;
conveying the pulse of light from the light source to the tissue surface via the light delivery element, the pulse of light being absorbed by blood vessels in the tissue, such that at least a portion of the tissue surface is damaged, and such that a portion of the blood vessels under the tissue surface are coagulated; and
applying a therapeutic substance to surface of the treatment area.
